(19) United States
(12) Patent Application Publication
(10) Pub. No.: US 2026/0242775 A1
(43) Pub. Date: Aug. 20, 2026

(54) In Situ Library Preparation and Cell Barcoding Methods in Partitions

(71) Applicant: Factorial Diagnostics, Inc., San Carlos, CA (US)

(72) Inventors: Katie Leigh Zobeck, Sunnyvale, CA (US); John Daniel Wells, San Francisco, CA (US)

(21) Appl. No.: 19/159,303

(22) PCT Filed: Feb. 23, 2024
(86) PCT No.: PCT/US2024/017178

§ 371 Date: Aug. 22, 2025

Related U.S. Application Data
(60) Provisional application No. 63/486,599, filed on Feb. 23, 2023.
(60) Provisional application No. 63/506,221, filed on Jun. 5, 2023.
(60) Provisional application No. 63/506,338, filed on Jun. 5, 2023.
(60) Provisional application No. 63/583,124, filed on Sep. 15, 2023.

(51) Int. Cl. C12N 15/10; C12Q 1/6806; C12Q 1/6855; C12Q 1/686; C12Q 1/6869

(52) U.S. Cl. CPC C12N 15/1082; C12N 15/1065; C12Q 1/6806; C12Q 1/6855; C12Q 1/686; C12Q 1/6869

(57) ABSTRACT

This disclosure provides methods for in situ library preparation, and in situ cell barcoding methods in partitions.

### 1. CROSS-REFERENCE TO RELATED APPLICATIONS

This application claims the benefit of U.S. Provisional Application Nos. 63/486,599, filed Feb. 23, 2023, 63/506,221, filed Jun. 5, 2023, 63/506,338, filed Jun. 5, 2023, 63/583,124, filed Sep. 15, 2023, which are incorporated by reference in their entireties.

### 2. BACKGROUND

Aspects of the present disclosure relates to methods and kits for cellular sample pooling before in situ library preparation of precursor libraries. Such methods can be used for droplet-based technologies, or for non-droplet-based technologies, such as in situ library preparation or in situ cell barcoding.

Aspects of the present disclosure also relate to methods and kits for library preparation and cell barcoding in partitions, such as, but not limited to, droplets. Such a method can be used for droplet-based technologies, such as for droplet-based sequencing methods.

### 3. SUMMARY

An aspect of the present disclosure includes a method of preparing in situ libraries, the method comprising: preparing, in situ, precursor libraries from a sample comprising a cell population to produce a precursor library within each cell, comprising nucleic acid fragments with a genomic region of interest and adapter sequences; preparing a mixture in one or more containers comprising one or more of: the cell population, cell barcoding oligonucleotides, and amplification reagents; partitioning the mixture into a plurality of partitions, wherein at least some partitions contain: a cell from the cell population comprising the precursor library, and a plurality of cell barcoding oligonucleotides; barcoding the precursor libraries in the plurality of partitions by: amplifying the cell barcoding oligonucleotides in the at least some partitions to produce cell barcoding primers; amplifying the precursor libraries in partitions with the cell barcoding primers to produce cell barcoded libraries; and isolating the cell barcoded libraries.

In some embodiments, a cell from the cell population can include a single cell. In some embodiments, a cell from the cell population can include one or more cells.

In some embodiments, the partition in the plurality of partitions is a droplet. In some embodiments, the partition in the plurality of partitions is an emulsion. In some embodiments, the partition in the plurality of partitions is a container.

In some embodiments, the container is a well. In some embodiments, the well is a microwell or nanowell. In some embodiments, the partition is a hydrogel. In some embodiments, the partition is poly(ethylene glycol) (PEG).

In some embodiments, said partitioning the mixture into the plurality of partitions occurs within a single stream from a single container. In some embodiments, said partitioning the mixture into the plurality of partitions occurs after the mixture from two containers are combined.

In some embodiments, each partition in the plurality of partitions holds a volume ranging from 0.1 to 5 nanoliters. In some embodiments, each partition in the plurality of partitions has a volume ranging from 0.1 to 1 nanoliters. In some embodiments, the droplet has a volume ranging from 0.1 to 5 nanoliters.

In some embodiments, the at least some partitions comprise at least 80% partitions containing cells from the cell population comprising the precursor library and the plurality of cell barcoding oligonucleotides. In some embodiments, the at least some partitions comprise at least 70% partitions containing cells from the cell population comprising the precursor library and the plurality of cell barcoding oligonucleotides. In some embodiments, at least some partitions comprise at least 60% partitions containing cells from the cell population comprising the precursor library and the plurality of cell barcoding oligonucleotides. In some embodiments, at least some partitions comprise at least 50% partitions containing cells from the cell population comprising the precursor library and the plurality of cell barcoding oligonucleotides. In some embodiments, the at least some partitions comprise at least 30% partitions containing cells from the cell population comprising the precursor library and the plurality of cell barcoding oligonucleotides. In some embodiments, the at least some partitions comprise at least 20% partitions containing cells from the cell population comprising the precursor library and the plurality of cell barcoding oligonucleotides.

In some embodiments, said amplifying the cell barcoding oligonucleotides comprises amplifying via isothermal amplification.

In some embodiments, wherein amplifying the precursor libraries in partitions with the cell barcoding primers to produce cell barcoded libraries comprises PCR amplification. In some embodiments, the PCR amplification is digital PCR amplification. In some embodiments, at least one of the amplification reagents comprise a buffer. In some embodiments, at least one of the amplification reagents comprise a PCR polymerase enzyme. In some embodiments, at least one of the amplification reagents comprise an isothermal polymerase enzyme.

In some embodiments, at least one of the amplification reagents comprise one or more polymerase enzymes selected from: Klenow Fragment (Exo-), Bsu Large Fragment, Bst DNA polymerase, Bst2.0, Sequenase, Bsm DNA Polymerase, EquiPhi29, and Phi29 DNA polymerase. In some embodiments, the at least one amplification reagents comprises one or more enzymes selected from: DNA polymerase, RNA polymerase, nicking enzyme, a Bst2.0 polymerase, a Phi29 polymerase, and a DNA ligase.

In some embodiments, the at least one amplification reagents comprise dNTP and MgSO4. In some embodiments, the at least one amplification reagents comprise amplification oligonucleotides. In some embodiments, amplifying via isothermal amplification and amplifying via PCR amplification in the partition occur in a single reaction container comprising the buffer. In some embodiments, the buffer is not aspirated, washed, or modified between isothermal amplification and PCR amplification steps. In some embodiments, isothermal amplification and PCR amplification occurs in a single reaction container.

In some embodiments, the method further comprises, before barcoding the precursor libraries in the plurality of partitions, lysing the cell in each cell population.

In some embodiments, the method further comprises, after barcoding the precursor libraries in the plurality of partitions, lysing the cell in each cell population. In some embodiments, preparing precursor libraries comprises: performing, in each cell of the cell population, an enzymatic fragmentation reaction to form nucleic acid fragments; ligating, in each cell, the nucleic acid fragments to adapter sequences in situ to create a precursor library comprising ligated nucleic acid fragments.

In some embodiments, before preparing precursor libraries, the method comprises mixing a second cell population with the cell population to create a cell population mixture. In some embodiments, the method comprises, before enzymatic fragmentation, the method comprises fixing or permeabilizing the first cell population.

In some embodiments, the method comprises, before enzymatic fragmentation, the method comprises fixing or permeabilizing the second cell population. In some embodiments, before preparing precursor libraries, the method comprises mixing a fixed or permeabilized second cell population with the fixed or permeabilized cell population to create a cell population mixture.

In some embodiments, further comprising fixing or permeabilizing the cell population mixture before preparing precursor libraries. In some embodiments, the method further comprises performing a heat denaturation step on the cells before enzymatic fragmentation. In some embodiments, further comprising washing the cell population mixture after the heat denaturation step with a buffer.

In some embodiments, further comprising at least a second washing step to wash the population mixture with the buffer. In some embodiments, before mixing the second cell population with the first cell population to create the cell population mixture: introducing a first set of unique well identifiers (UWIs) to the first population of cells; introducing a second set of UWIs to the second population of cells; wherein the first set of UWIs comprise one or more barcode reads and a non-genomic fragment and the second set of UWIs comprise one or more barcode reads and a non-genomic fragment; preparing, in situ, the precursor library from the first and second population of cells to produce the precursor library, the precursor library comprising nucleic acid fragments comprising a genomic region of interest from the first and second cell populations and one or more adapter sequences; barcoding, in situ, the precursor library and the first and second set of UWIs to produce: a cell barcoded precursor library; a first and second set of cell barcoded UWIs; isolating the cell barcoded precursor library and cell barcoded UWIs; sequencing the cell barcoded precursor library and cell barcoded UWIs; and analyzing sequencing reads to identify: cells belonging to the first population of cells and cells belonging to the second population of cells based on the UWI.

In some embodiments, analyzing comprises analyzing the cell barcoded UWI by the non-genomic fragment of the UWI. In some embodiments, said isolating the cell barcoded libraries comprises recovering amplicons from the cell barcoded libraries of the at least some partitions. In some embodiments, isolating the cell barcoded libraries comprises breaking the partition.

In some embodiments, isolating the cell barcoded libraries comprises lysing the cell in the partition. In some embodiments, the concentration of barcoding oligonucleotides in the at least some partitions ranges from 25 to 600 pM. In some embodiments, the concentration of barcoding oligonucleotides in the at least some partitions ranges from 45 pM to 550 pM. In some embodiments, the concentration of barcoding oligonucleotides in the at least some partitions ranges from 50 nM to 500 nM. In some embodiments, the concentration of barcoding oligonucleotides comprises 5 pM to 500 pM.

In some embodiments, the amount of barcoding oligonucleotides in each partition containing the cell ranges from 50 barcoding oligonucleotides to 8.5 million barcoding oligonucleotides.

An aspect of the present disclosure includes a method for cellular sample pooling, the method comprising: introducing a first set of unique well identifiers (UWIs) to a first population of cells, the first set of UWIs comprising one or more barcode reads and a non-genomic fragment; introducing a second set of UWIs to a second population of cells, the second set of UWIs comprising one or more barcode reads and a non-genomic fragment; mixing the first and second population of cells; preparing, in situ, a precursor library from the first and second population of cells to produce the precursor library, the precursor library comprising nucleic acid fragments comprising a genomic region of interest and one or more adapter sequences; barcoding, in situ, the precursor library and the first and second set of UWIs to produce: a cell barcoded precursor library, a first and second set of cell barcoded UWIs; isolating the cell barcoded precursor library and cell barcoded UWIs; sequencing the cell barcoded precursor library and cell barcoded UWIs; and analyzing sequencing reads to identify: cells belonging to the first population of cells and cells belonging to the second population of cells based on the UWI.

An aspect of the present disclosure includes a kit comprising: one or more microfluidic chips; reagents for preparing precursor libraries in situ comprising: a fragmentation enzyme and buffer for performing an enzymatic fragmentation reaction to form one or more nucleic acid fragments within a cell; an End repair and A tail (ERA) master mix and buffer for performing an end-repair and A-tailing reaction on the one or more nucleic acid fragments; a ligation enzyme and buffer; adapter sequences, wherein the ligation enzyme and buffer, and adapter sequences are capable of ligating, in each cell, the nucleic acid fragments to the adapter sequences in situ to create a ligated library comprising ligated nucleic acid fragments; amplification primers for amplifying the ligated nucleic acid fragments to form amplicon products; a polymerase chain reaction (PCR) enzyme master mix comprising one or more of: an enzyme, a buffer, or an enzyme and a buffer; in an amount sufficient to prepare a precursor library in situ; reagents for cell barcoding comprising: cell barcoding oligonucleotides comprising: an oligonucleotide, wherein the oligonucleotide comprises: an amplification sequence, and a consensus region that is complementary to a target sequence of a nucleic acid fragment; and a second oligonucleotide, wherein the second oligonucleotide comprises: a second amplification sequence, a second consensus region that complementary to a target sequence of a nucleic acid fragment; amplification reagents comprising: a first amplification primer comprising a nucleotide sequence that is complementary to the amplification sequence on the oligonucleotide; a second amplification primer comprising a nucleotide sequence that is complementary to the second amplification sequence on the second oligonucleotides; and an isothermal amplification polymerase; instructions for carrying out the precursor library preparation in situ; instructions for preparing a mixture in one or more containers comprising one or more of: the cell population, cell barcoding oligonucleotides, and amplification reagents; instructions for partitioning the mixture into a plurality of partitions; instructions for barcoding the precursor libraries in the plurality of partitions; instructions for isolating the cell barcoded libraries; and instructions for sequencing the cell barcoded libraries.

In some embodiments, instructions for carrying out the precursor library comprises the following steps: performing, in each cell of the cell population, an enzymatic fragmentation reaction to form nucleic acid fragments; ligating, in each cell, the nucleic acid fragments to adapter sequences in situ to create a precursor library comprising ligated nucleic acid fragments.

In some embodiments, the instructions for preparing the mixture comprises the following steps: preparing the mixture in one or more containers comprising one or more of: the cell population, cell barcoding oligonucleotides, and amplification reagents.

In some embodiments, instructions for partitioning the mixture into a plurality of partitions comprises the following steps: partitioning the mixture into a plurality of partitions, wherein at least some partitions contain: a cell from the cell population comprising the precursor library, and a plurality of cell barcoding oligonucleotides.

In some embodiments, instructions for barcoding the precursor library in a plurality of partitions comprises the following steps: barcoding the precursor libraries in the plurality of partitions by: amplifying the cell barcoding oligonucleotides in the at least some partitions to produce cell barcoding primers; amplifying the precursor libraries in partitions with the cell barcoding primers to produce cell barcoded libraries.

In some embodiments, further comprising one or more buffers. In some embodiments, further comprising a cell lysis buffer. In some embodiments, the amplification primers comprise barcoding primers, sequencing primers, or a combination thereof.

In some embodiments, the kit further comprises protease K. In some embodiments, the kit further comprises barcoding primers, and a second PCR Enzyme master mix comprising one or more of: an enzyme, a buffer, or an enzyme and a buffer. In some embodiments, the kit further comprises a lytic enzyme. In some embodiments, further comprising a thermocycler.

In some embodiments, further comprising a partitioning instrument for partitioning cells, cell barcoding oligonucleotides, or a combination of cells and cell barcoding oligonucleotides. In some embodiments, further comprising an instrument comprising a thermocycler and a partition engine.

In some embodiments, the partition engine is configured to hold one or more microfluidic chips. In some embodiments, the one or more microfluidic chips is configured to hold a sample comprising one or more cells in the cell population, amplification reagents, and one or more cell barcoding oligonucleotides.

In one aspect, this disclosure features a method of pre-preparation of in situ ligation libraries before droplet-based isolation.

In another aspect, this disclosure features a method of droplet based in situ ligation prep and bulk PCR.

In another aspect, this disclosure features a method of droplet based in situ ligation prep and droplet merging.

In another aspect, this disclosure features a sample pooling method that can be used for droplet-based technologies, non-droplet-based technologies, such as in situ library preparation methods for cell barcoding, and RNA or DNA library preparations of various methods.

### 4. BRIEF DESCRIPTION OF THE DRAWINGS

FIG. 1 provides a schematic of an overview of sample well pooling and read identification.

FIG. 2 provides a schematic showing integration of well barcoding with in situ ligation library preparation of precursor libraries containing a target genomic region of interest and one or more adapter sequences, and single cell sequencing.

FIG. 3 provides a schematic showing integration of well barcoding with 10X single cell ATACseq chemistry.

FIG. 4 provides a schematic showing methods of preparing precursor in situ libraries, and subjecting the precursor libraries with cell barcodes with droplets, followed by droplet-based sequencing.

FIG. 5 shows a micrograph of in situ library prepped cells (precursor library) within droplets.

FIG. 6 shows cell barcoded libraries (amplicon products) recovered after performing a stream partitioning reaction in droplets, followed by a single reaction cell barcoding amplification method in droplets.

FIG. 7 shows cell barcoded libraries (amplicon products) recovered after performing a single stream partitioning reaction in droplets, followed by a single reaction cell barcoding amplification method in droplets.

FIG. 8 provides sequencing analysis of droplet cell barcoded libraries.

FIG. 9 provides sequencing analysis of droplet cell barcoded libraries.

FIG. 10 provides an example of amplicon products recovered after single reaction isothermal amplification and PCR amplification steps during cellular barcoding in partitions. Different buffers and polymerase enzymes were tested.

FIG. 11 shows the effect of a heat denaturation step during library preparation on supernatant DNA when the cells were fixed with paraformaldehyde (PFA).

FIG. 12 shows an example of in situ library preparation and cellular barcoding in droplets following Protocol G in the examples, where little separation between mouse and human cells were observed, likely due to the cell fixation type and method performed (“Protocol A” in the examples).

### 5. DETAILED DESCRIPTION

### Methods

Aspects of the present disclosure include methods of preparing, in situ, nucleic acid libraries in partitions. Such a method can include preparation of precursor libraries, and barcoding the precursor libraries to produce cell barcoded libraries, in partitions.

This disclosure features methods where precursor ligation libraries can be prepared in situ within a bulk reaction before partitioning the cells in a partition.

### Partitions

“Partitions” as used herein, refers to means of separating a cell from another cell, and means of holding or encapsulating one or more cells. In some embodiments, a partition is differentiated from another partition. In some embodiments, the partition is a cell (e.g., whole cells or nuclei) or nucleus. Cells can be intact in a partition, or cells can be lysed or disrupted in a partition for availability of nucleic acid fragments (e.g., precursor libraries) within the cell.

In some embodiments, a partition is a droplet. Each partition, such as a droplet, containing a cell can then be merged with zero, one, or more additional partitions such that the partition is capable of labeling the cell with a unique cell barcode or barcode combination during a cell barcoding step.

In some embodiments, a partition is an emulsion. In some embodiments, a partition is a container. In certain embodiments, the partition is a well, a microwell, or a nanowell. In certain embodiments, the partition is a hydrogel. In certain embodiments, the partition is poly(ethylene glycol) (PEG). For example, with a partition is PEG, a cell containing precursor libraries is encapsulated in PEG. In some embodiments, the partition is a molecular crowding agent.

The method includes preparing, in situ, precursor libraries from a sample comprising one or more cells. As used herein, the term “precursor library”, refers to nucleic acid (e.g., DNA or RNA) fragments with a genomic region of interest and adapter sequences with in intact cells.

In some embodiments, the sample comprises a cell population, and the method includes preparing, in situ, precursor libraries from the sample comprising a cell population to produce a precursor library within each cell.

### In Situ Library Preparation to Produce Precursor Libraries

This disclosure describes a mechanism to utilize in situ library prep (see, e.g., International Patent Application Nos. PCT/US2021/046025 and PCT/US2023/062776, each of which are hereby incorporated by reference in their entireties) to produce precursor libraries. Precursor libraries within a cell include two distinct universal sequences, one on each end of a nucleic acid fragment. The nucleic acid fragment can be DNA or RNA.

In some embodiments, in situ library prep can be performed by methods other than those described in PCT/US2021/046025 and PCT/US2023/062776. One non-limiting example is an in situ library preparation method that is compatible with the methods described herein include an assay for transposase-accessible chromatin with sequencing (ATAC-seq). ATAC-seq uses tagmentation to add a universal sequence on two sides of genomic DNA.

Another example of an in situ library preparation method to create precursor libraries is compatible with the methods described herein include methods that create cDNA from RNA, where the result cDNA contains at least one universal sequence on the insert transcript.

When using in situ library preparation with the methods described herein, the following compositions are the result of the in situ library preparation, denoted as a “precursor library”: Composition 1: 5′-UniversalSequence1-GenomicDNA-Complement to UniversalSequence2-3′ and Composition 2: 5′-UniversalSequence1-cDNA-Complement to UniversalSequence2-3′

In some embodiments, the precursor library is a DNA amplicon product.

In some embodiments, the precursor library is a DNA product of ligation.

In some embodiments, the precursor library is a DNA product of tagmentation.

In some embodiments, the precursor library comprises genomic DNA (gDNA) modified to contain a first consensus read region at the 5′ end of the DNA sequence and a second consensus read region at the 5′ end of the DNA sequence.

In some of the described methods (see, e.g., Option 2 described below) could also be performed with a library containing only one Universal sequence attached to the gDNA or cDNA. In such cases, the following compositions are the result of the in situ library preparation: Composition 3: 5′-GenomicDNA-Complement to UniversalSequence2-3′ and Composition 4: 5′-cDNA-Complement to UniversalSequence2-3′.

In some embodiments, the methods described here can be used with any size of genomic DNA or cDNA input material. In some embodiments, the methods described herein are compatible with any existing sequencing methods.

In some embodiments, the methods described herein can be used with short read sequencing insert sizes. In some embodiments, the short read sequencing insert sizes are between about 50 bp to about 600 bp (or any of the ranges therein). In some embodiments, the methods described herein can be used with long read sequencing insert sizes. In some embodiments, the long read sequencing insert sizes are at least 1 kb, at least 2 kb, at least 3 kb, at least 4 kb, at least 5 kb, at least 6 kb, at least 7 kb, at least 8 kb, at least 9 kb, or at least 10 kb.

In some embodiments, the molecules described in the compositions can be single strand, or double strand, or partially single and double strand (i.e. contains a Y-adapter at one or both ends).

### Steps of Library Preparation

Fixing and/or Permeabilizing the Cells

In some embodiments, the method includes preparing precursor libraries from a cell before partitioning the cells. In some embodiments, the method includes preparing precursor libraries from a cell after partitioning the cells.

In some embodiments, the first step of library preparation includes fixing and/or permeabilizing the cells.

Fixation occurs when cells are incubated in the presence of a fixing agent. Fixing the cellular sample can be performed by any convenient method as desired. Fixing the cellular sample can also include permeabilizing the cell membrane. For example, in some embodiments, the cellular sample is fixed according to fixing and permeabilization techniques described in U.S. Pat. No. 10,627,389, which is hereby incorporated by reference in its entirety.

In some embodiments, fixing the cellular sample includes contacting the sample with a fixation reagent. Fixation reagents of interest are those that fix the cells at a desired time-point. Any convenient fixation reagent may be employed, where suitable fixation reagents include, but are not limited to: glutaraldehyde, formaldehyde, paraformaldehyde, formaldehyde/acetone, methanol/acetone, ethanol etc. For example, paraformaldehyde used at a final concentration of about 1 to 15% has been found to be a good cross-linking fixative. In some embodiments, the fixation reagent is a mixture of fixatives. Non-limiting examples of fixative mixtures include, IncellMax (IncellDx, Inc), Bouin, Clarke solution, Carnoy, and formaldehyde solutions. In some embodiments, multiple fixative agents are used consecutively. In some embodiments, multiple fixative agents are used simultaneously.

In some embodiments, the cells in the sample are permeabilized by contacting the cells with a permeabilizing reagent. Permeabilizing reagents of interest are reagents that allow the labeled biomarker probes, e.g., as described in greater detail below, to access to the intracellular environment. Any convenient permeabilizing reagent may be employed, where suitable reagents include, but are not limited to: mild detergents, such as EDTA, Tris, IDTE (10 mM Tris, 0.1 mM EDTA), Triton X-100, NP-40, saponin, digitonin, leucoperm, Tween-20, etc.; methanol, and the like. In some embodiments, the fixing agent will also permeabilize cells. Examples of fixing agent which can also permeabilize cells include, but are not limited to, acetone, methanol, and IncellMax (IncellDx, Inc).

In certain embodiments, the cells are incubated in the presence of a fixing agent for about 5 minutes. In certain embodiments, the cells are incubated in the presence of a fixing agent for about 10 minutes. In certain embodiments, the cells are incubated in the presence of a fixing agent for about 15 minutes. In certain embodiments, the cells are incubated in the presence of a fixing agent for about 20 minutes. In certain embodiments, the cells are incubated in the presence of a fixing agent for about 30 minutes. In certain embodiments, the cells are incubated in the presence of a fixing agent for about 1 hour.

In specific embodiments, the fixing agent is formaldehyde or paraformaldehyde. In some embodiments, the cells are incubated in the presence of formaldehyde or paraformaldehyde for about 10 minutes. In certain embodiments, the cells are incubated in the presence of formaldehyde or paraformaldehyde for about 15 minutes. In certain embodiments, the cells are incubated in the presence of formaldehyde or paraformaldehyde for about 20 minutes. In certain embodiments, the cells are incubated in the presence of formaldehyde or paraformaldehyde for about 30 minutes. In certain embodiments, the cells are incubated in the presence of formaldehyde or paraformaldehyde for about 1 hour.

In specific embodiments, the fixing agent is methanol. In some embodiments, the cells are incubated in the presence of methanol for about 10 minutes. In certain embodiments, the cells are incubated in the presence of methanol for about 15 minutes. In certain embodiments, the cells are incubated in the presence of methanol for about 20 minutes. In certain embodiments, the cells are incubated in the presence of methanol for about 30 minutes.

### Ligation Based Library Preparation

### Enzymatic Fragmentation

In some embodiments, the next step of preparing precursor libraries includes performing, in each cell of a cell population, an enzymatic fragmentation reaction to form nucleic acid fragments within the cell.

In some embodiments, the method includes contacting the cells with a fragmentation buffer and a fragmentation enzyme to form an enzymatic fragmentation mixture. Performing an enzymatic fragmentation reaction in the present ligation-based method provides for generating smaller sized nucleic acid fragments containing the target region of interest. Methods for fragmenting nucleic acid can include mechanical, chemical, or enzyme-based fragmenting. Mechanical shearing methods include acoustic shearing, sonication, hydrodynamic shearing and nebulization. Chemical fragmentation methods include the use of agents which generate hydroxyl radicals for random DNA cleavage or the use of heat with divalent metal cations, while enzyme-based methods include transposases, restriction enzymes (e.g. mung bean nucleases, nuclease P1, or micrococcal nuclease), DNase I, non-specific nucleases, and nicking enzymes, or a mixture thereof. In some embodiments, enzyme-based DNA/RNA fragmentation methods include using a mixture of at least two different enzymes e.g. two or more of the enzymes mentioned in the preceding sentence e.g. two or more nucleases, Any standard enzymatic fragmentation buffer and enzymatic fragmentation enzyme can be used for fragmenting the nucleic acid.

In certain embodiments, the method optionally includes denaturing, by heat, prior to enzymatic fragmentation to improve fragmentation, likely by opening the chromatin structure of nucleic acid in the cells. In some embodiments, the method includes performing a heat denaturation step prior to enzymatic fragmentation.

In alternative embodiments, the heat denaturation step is not performed prior to enzymatic fragmentation.

In certain embodiments, the enzymatic fragmentation mixture does not include EDTA. In certain embodiments, the enzymatic fragmentation mixture includes EDTA.

In some embodiments, the fragmentation enzyme is selected from a KAPA fragmentation enzyme, TaKara fragmentation enzyme, NEBNext Ultra enzymatic fragmentation enzyme, biodynamic DNA Fragmentation Enzyme Mix, KAPA Fragmentation Kit for Enzymatic Fragmentation, SureSelect Fragmentation enzyme, Ion Shear™ Plus Enzyme, and the like. In some embodiments, the fragmentation enzyme is a Caspase-Activated DNase (CAD). In some embodiments, a fragmentation enzyme and fragmentation buffer are contacted with the cells in an amount sufficient to perform a fragmentation reaction.

In some embodiments, the fragmentation buffer is selected from a KAPA fragmentation buffer, TaKara fragmentation buffer, NEBNext Ultra enzymatic fragmentation buffer, biodynamic DNA Fragmentation buffer, KAPA Fragmentation buffer, SureSelect Fragmentation Buffer, Ion Shear™ Plus Reaction Buffer, and the like. However, any commercially available enzymatic fragmentation buffer can be used for fragmenting the nucleic acid of the cells.

In some embodiments, the enzymatic fragmentation mixture comprises a conditioning solution. In some embodiments, the volume of conditioning solution added to the enzymatic fragmentation mixture ranges from 1 μl to 20 μl. In some embodiments, the conditioning solution is a solution that adjusts the enzymatic fragmentation buffer to handle highly sensitive reagent compositions, and in some cases sequesters EDTA (or other chelators) in the sample. In some embodiments, the conditioning solution contains a reagent that binds EDTA in the sample. In some embodiments, the conditioning solution contains Magnesium or other cations to bind to EDTA in the cell population. In some embodiments, the conditioning solution is a solution that binds to magnesium in the sample. In some embodiments, the conditioning solution contains a divalent cation chelator to bind to excess magnesium in the sample.

In some embodiments, the method includes performing enzymatic fragmentation on the nucleic acids (e.g., DNA or RNA) within the cell to form an enzymatic fragmentation reaction mixture. In some embodiments, performing an enzymatic fragmentation reaction on the mixture comprises loading the enzymatic fragmentation mixture into a suitable temperature-control device (although, in some such embodiments: (a) the mixture contains fewer than 15,000 fixed cells, or from 17,000-79,000 fixed cells, or more than 81,000 fixed cells; and/or (b) the temperature-control device maintains the temperature at from 15-36° C. or from 38-45° C. during the fragmentation reaction; and/or (c) for fewer than 59 minutes). In some embodiments, performing an enzymatic fragmentation reaction on the mixture comprises loading the enzymatic fragmentation mixture onto a thermocycler. In some embodiments, performing an enzymatic fragmentation reaction on the mixture comprises loading the enzymatic fragmentation mixture onto a heat block. In some embodiments, performing an enzymatic fragmentation reaction on the mixture comprises loading the enzymatic fragmentation mixture into a water bath. In some embodiments, performing an enzymatic fragmentation reaction on the mixture comprises loading the enzymatic fragmentation mixture into an incubator.

In some embodiments, the method includes incubating the enzymatic fragmentation mixture in the temperature control device (e.g. thermocycler) for a duration/time period ranging from 1 minute to 120 minutes. In some embodiments, before fragmenting, the method includes a pre-incubation step to allowing the enzymes to enter the cell.

In some embodiments, performing an enzymatic fragmentation reaction on the mixture comprises loading the mixture onto a temperature control device (e.g. thermocycler) and incubating the mixture at a temperature ranging from 2° C. to 80° C. In some embodiments, performing an enzymatic fragmentation reaction on the mixture comprises loading the mixture onto a temperature-control device (e.g. thermocycler or heat-block) and incubating the mixture at a temperature of 14-20° C. In some embodiments, performing an enzymatic fragmentation reaction on the mixture comprises loading the mixture onto a temperature-control device (e.g. thermocycler or heat-block) and incubating the mixture at a temperature of 20-30° C. In some embodiments, performing an enzymatic fragmentation reaction on the mixture comprises loading the mixture onto a temperature-control device (e.g. thermocycler or heat-block) and incubating the mixture at a temperature 35-38° C.

In some embodiments, before the ligating step (c) of the ligation-based method, the method includes performing an end-repair and/or A-tailing reaction on the one or more nucleic acid fragments. In some embodiments the enzymatic fragmentation enzyme is heat inactivated before end repair and A (ERA) tailing (described below) at a known temperature for inactivating the specific enzyme 65-99.5° C. for 5-60 minutes. In some embodiments the End repair and A tailing incubation step also acts as the heat inactivation step for enzymatic fragmentation enzymes.

In some embodiments, the End-repair and A-tailing reaction and the enzymatic fragmentation reaction occurs in a single reaction, with multiple temperature incubations. For example, the End repair and/or A-tailing reaction can occur during the enzymatic fragmentation reaction in a single reaction. In some embodiments the End repair reaction can occur at a certain temperature. Subsequently, A-tailing reaction can occur at a different temperature following a temperature change. In other embodiments, the End repair and/or A-tailing reaction can occur in different, separate reactions. In some embodiments, the End-repair and A-tailing reaction and the enzymatic fragmentation reaction are separate reactions.

### End Repair and A-tailing

In some embodiments, the next step of library preparation to produce precusor libraries includes performing an End-repair and/or A-tailing reaction on the one or more fragmented nucleic acid within the cell. End Repair and/or A-Tailing are two enzymatic steps configured to blunt the nucleic acid fragments and, optionally, add an overhanging A nucleotide to the end of the nucleic acid fragments, for example, to improve ligation efficiency. The end-repair and/or A-tailing reaction is performed before ligating the nucleic acid fragments.

In some embodiments, the End Repair (ER) and/or A-tailing can occur in the same reaction as the enzymatic fragmentation reaction described above. In certain embodiments, the end repair and/or A-tailing occurs in the same reaction as the enzymatic fragmentation. In some embodiments, the method comprises mixing the cell with an enzyme fragmentation, ER, and A-tailing enzyme cocktail mixture. In some embodiments, the cocktail mixture comprises a concentration that is at least 0.125X, 0.5X, 1X, 1.5X, 2X, 2.5X, 3X, 3.5X, 4X, 4.5X, 5X, 5.5X, 6X, 6.5X, 7X, 7.5X, 8X, 8.5X, 9X, 9.5X, or 10X the manufactured recommended enzyme concentrations (Watchmaker Genomics). In some embodiments, the enzyme fragmentation, ER, and A-tailing enzyme are included in concentrations suitable for producing appropriate library fragment sizes.

In some embodiments, performing an end-repair and/or A-tailing reaction comprises contacting the fragmented nucleic acid within the cell with an End Repair A-tail buffer and an End Repair A-tail enzyme to form an End Repair A-tail mixture. In some embodiments, performing an End-repair and A-tailing reaction comprises contacting the fragmented nucleic acid within the cell in the enzymatic fragmentation reaction mixture with an End Repair A-tail buffer and an End Repair A-tail enzyme to form an End Repair A-tail mixture. In some embodiments, contacting the fragmented nucleic acid within the cell in the enzymatic fragmentation reaction mixture with an End Repair A-tail buffer and an End Repair A-tail enzyme at a temperature ranging from 1° C. to 10° C. In some embodiments, contacting the fragmented nucleic acids within the cell in the enzymatic fragmentation reaction mixture with an End Repair A-tail buffer and an End Repair A-tail enzyme occurs on ice. The temperature may then be increased for enzymatic reactions to occur e.g. to from 25-40° C.

In some embodiments, the method further comprises running the End Repair A-tail mixture in a thermocycler to form an End Repair A-tail reaction mixture.

In some embodiments, the End Repair A-tail mixture is incubated in the thermocycler at a temperature ranging from 2° C. to 90° C. In some embodiments, performing an End Repair A-tail reaction on the End Repair A-tail mixture comprises loading the End Repair A-tail mixture onto a thermocycler and incubating the End Repair A-tail mixture at a temperature ranging from 2° C. to 50° C., such as 4° C. to 37° C., 4° C. to 50° C., or 5° C. to 40° C.

In some embodiments, the End Repair A-tail mixture is incubated for a duration ranging from 5 minutes to 50 minutes. In some embodiments the End repair and A tail enzymes are heat inactivated before proceeding to ligation at 65-100° C. for 5-60 minutes or more.

### Optimization of Enzymatic Fragmentation and A-Tailing

In some embodiments, the enzymatic fragmentation step is optimized for the cells conjugated with magnetic beads. In some of these embodiments, enzymatic fragmentation and A-tailing enzymes are added separately to the enzymatic fragmentation reaction. In some of these embodiments, enzymatic fragmentation and A-tailing enzymes are added together to the enzymatic fragmentation reaction.

In various embodiments, the enzymatic fragmentation reaction is performed for no less than 10 min, such as no less than 15 min, no less than 20 min, no less than 30 min, or no less than 40 min. In specific embodiments, the fragmentation reaction is performed for about 15 min, about 20 min, about 30 min, about 40 min, or about 50 min.

In specific embodiments, the enzymatic fragmentation and A-tailing enzymes used in the enzymatic fragmentation reaction are about 1.5 times, about 2 times, about 2.5 times, or about 3 times of the manufacturer's recommendation. In some embodiments, the ligation step has a reaction volume of 5 μL to 75 μL, such as 5 μL to 50 μL, 5 μL to 30 μL, 5 μL to 25 μL, 5 μL to 20 μL, 5 μL to 15 μL, or 5 μL to 10 μL.

In various embodiments, the ligated nucleic acid fragments have a size of 100 bp to 1000 bp, such as 200 bp to 800 bp, 200 bp to 600 bp, 200 bp to 400 bp, 200 bp to 300 bp, 300 bp to 800 bp, 300 bp to 600 bp, 300 bp to 400 bp, 400 bp to 800 bp, or 400 bp to 600 bp. In certain embodiments, the ligated nucleic acid fragments have a size about 100 bp, about 150 bp, about 200 bp, about 250 bp, about 300 bp, about 350 bp, about 400 bp, about 500 bp, about 600 bp, about 800 bp, or about 1000 bp.

### Ligation

In some embodiments, the next step of preparing precursor libraries includes ligating, in each cell, the nucleic acid fragments to adapter sequences to create a ligated library comprising ligated nucleic acid fragments.

The ligation-based library preparation method includes ligating, in each cell, the nucleic acid fragments to adapter sequences in situ to create a ligated library comprising ligated nucleic acid fragments (e.g. precursor libraries).

Ligation adapter sequences may include modifications such as: methylation, capping, 3′-deoxy-2′,5′-DNA, N3′ P5′ phosphoramidates, 2′-O-alkyl-substituted DNA, 2′-O-methyl DNA, 2′ Fluoro DNA, Locked Nucleic Acids (LNAs) with 2′-O-4′-C methylene bridge, inverted T modifications (e.g. 5′ and 3′), or PNA (with such modifications at one or more nucleotide positions). Ligation adapter sequences may also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters).

In some embodiments, ligating includes performing ligase chain reaction (LCR). The ligase chain reaction (LCR) is an amplification process that involves a thermostable ligase to join two probes or other molecules together. In some embodiments, the thermostable ligase can include, but is not limited to, Pfu ligase, Taq ligase, HiFi Taq DNA ligase, 9°N DNA ligase, Thermostable 5′ AppDNA/RNA ligase, Ampligase® ligase, or a T4 RNA ligase (e.g. T4 RNA ligase 2). In some embodiments, the ligated product is then amplified to produce an amplicon product. In some embodiments, LCR can be used as an alternative approach to PCR. In other embodiments, PCR can be performed after LCR.

Ligating the nucleic acid fragments to the adapter sequences comprises running the nucleic acid fragments and adapter sequences in a thermocycler at a temperature and duration sufficient to ligate the nucleic acid fragmented to the adapter sequences. Ligation reagents and/or enzymes can be used for ligating the nucleic acid fragments. In some embodiments, ligation chain reaction (LCR) can be used for ligating the nucleic acid fragments.

Ligation of fragments to adapter sequences can also be performed using ligation without LCR (e.g. without the use of thermal cycling). Adapters can be ligated enzymatically, using any suitable DNA/RNA ligase. For instance, ligation can use Pfu ligase from Pyrococcus furiosus, Taq ligase from Thermus aquaticus (e.g. HiFi Taq DNA ligase), DNA ligase from Cholorella virus (e.g. PBCV-1 DNA ligase), T4 DNA ligase, Quick ligase, Blunt/TA ligase, T3 bacteriophage DNA ligase, T7 bacteriophage DNA ligase, a DNA ligase from Thermococcus (e.g. 9°N DNA ligase), Thermostable 5′ AppDNA/RNA ligase, Ampligase® ligase, Instant Sticky End ligase, T4 RNA ligases (e.g. T4 RNA ligase 1, T4 RNA ligase 2 truncated, T4 RNA ligase truncated K227Q, and T4 RNA ligase 2 truncated KQ), or a RtcB ligase. Ligases which are able to be heat-inactivated are preferred. For example, ligases which can be heat inactivated through heating to 65° C. for 10 minutes are preferred.

The fragmented nucleic acid are contacted with adapter sequences to form a ligated library/ligation mixture containing the ligated nucleic acid fragments. In some embodiments, the ligation mixture can include a Ligation Master Mix. In some embodiments, the ligation mixture can include a Blunt/TA Ligase Master Mix, or an Instant Sticky End Ligase Master Mix.

Adapter Ligation enzymatically combines (e.g., ligates) adapters provided in the reaction to the prepared nucleic acid fragments. Non-limiting examples of adapter sequences include, but are not limited to, adapter nucleotide sequences that allow high-throughput sequencing of amplified or ligated nucleic acids. In some embodiments, the adapter sequences are selected from one or more of: a Y-adapter nucleotide sequence, a hairpin nucleotide sequence, a duplex nucleotide sequence, and the like. In some embodiments, the adapter sequences are for paired-end sequencing. In some embodiments, the adapter sequences include sequencing read primer sequences (e.g., R1, R2, i5, i7 etc.). In some embodiments, the adapter sequences include sample barcodes. Adapter sequences can be used in a ligation reaction of the disclosed method for the desired sequencing method used.

In some embodiments, the ligation mixture includes the End-repair A-tail reaction mixture or enzymatic fragmentation reaction mixture, a set of adapter sequences, and a ligation master mix. In certain embodiments, ligation mixture includes the End-repair A-tail reaction mixture or enzymatic fragmentation reaction mixture, a set of adapter sequences, nuclease free H2O, and a ligation master mix. In certain embodiments, the ligation mixture includes a final volume ranging from 10 μl to 200 μl.

In some embodiments, the method includes ligating the fragmented nucleic acids to the adapter sequences. In certain embodiments, ligating the fragmented nucleic acids to the adapter sequences comprises running the ligation mixture in the thermocycler at a temperature and duration sufficient to ligate the fragmented nucleic acids to the adapter sequences, such as, but not limited to: barcoding sequences, consensus read regions for sequencing, adapter sequences, or other indexing sequences for the sequencing method being used.

In some embodiments, the temperature ranges from 4° C. to 90° C. In some embodiments, the method includes incubating the ligation mixture at a temperature of 20+5° C. In some embodiments, the method includes incubating the ligation mixture at a temperature of about 20° C. In some embodiments, the duration ranges from 5 minutes to 4 hours.

In some embodiments the ligase enzyme is heat inactivated e.g. at a temperature ranging from 65-99.5° C. for a duration ranging from 5-60 minutes before proceeding to the next steps. In some embodiments, ligase enzymes do not need to be heat inactivated.

### Examples of Additional Amplification of Ligated Library

In some embodiments of the in situ ligation-based library preparation method, after the ligation step, but before segmentation, the method further comprises amplifying the ligated nucleic acids fragments to form amplicon products. Amplifying the ligated nucleic acid fragments allows for creating more copies of the nucleic acids fragments, reducing the likelihood of region drop out due to inefficiencies in purification and/or hybridization capture protocols. Additionally, the method allows for adding additional sequences such as adapter sequences with sample barcodes, and the like during amplification. In some embodiments, amplifying the ligated nucleic acids fragments to form amplicon products comprises contacting the ligated nucleic acids fragments with amplification primers (e.g., primers used to hybridize with sample DNA or RNA that define the region to be amplified, but can also include, barcoding primers, R1/R2 primers, other sequencing primers, and the like).

Additionally, multiple PCR reactions may be performed, for example, after ligation. All of these additional PCR steps could occur before cell lysis. Additional PCR steps can include adding additional components to a PCR reaction, with each addition defined as a “PCR step”. For example, adding targeting primers, followed by adding amplification primers can take place in two PCR reactions, e.g. two PCR steps or one PCR reaction, e.g., one PCR step. In some embodiments, one or more, two or more, three or more, four or more, five or more, six or more, seven or more, eight or more, nine or more, or ten or more distinct PCR reactions can be performed. In certain embodiments, two PCR reactions are performed between ligation and sequencing steps (e.g., after ligation, but before lysing). In certain embodiments, three PCR reactions are performed between ligation and sequencing steps (e.g., after ligation, but before lysing). In certain embodiments, four PCR reactions are performed between ligation and sequencing steps (e.g., after ligation, but before lysing). In certain embodiments, the PCR reactions are performed after ligation but before the lysing step. In certain embodiments, the PCR reactions are performed after ligation but before the lysing step.

When performing amplification after the ligation step, the method includes contacting the ligated library (e.g., adapter ligated DNA or RNA fragments) with primers. In some embodiments, the method includes amplifying the ligated library with primers containing minimal sequences (e.g., read 1, read 2 sequences, etc.). In some embodiments, the method includes amplifying the ligated library with primers including sample barcodes. In some embodiments, the method includes amplifying the ligated library with primers including the sequencing adapters, such as P5 and P7.

Primers may include modifications such as: methylation, capping, 3′-deoxy-2′,5′-DNA, N3′ P5′ phosphoramidates, 2′-O-alkyl-substituted DNA, 2′-O-methyl DNA, 2′ Fluoro DNA, Locked Nucleic Acids (LNAs) with 2′-O-4′-C methylene bridge, inverted T modifications (e.g. 5′ and 3′), or PNA (with such modifications at one or more nucleotide positions). Ligation adapter sequences may also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters).

In some embodiments, the method includes amplifying the adapter-ligated fragments (e.g., precursor library) to create more copies cellular barcoding. In some embodiments, the method includes amplifying the adapter-ligated fragments to add full length adapter sequences onto the adapter-ligated fragments, if necessary.

In some embodiments, after the ligating step to produce the ligated library, the method includes contacting the ligated library with an amplification mixture. In some embodiments, the amplification mixture comprises any readily available, standard amplification library mix or one or more components thereof, a set of amplification primers, and the adapter-ligated library. In some embodiments, the amplification mixture comprises a KAPA HiFi Hotstart Ready Mix (2X) or one or more components from the ready mix thereof, a set of amplification primers, and the adapter-ligated library. In some embodiments, the amplification mixture comprises a xGen Library Amplification Primer Mix or one or more components from the primer mix thereof, a set of amplification primers, and the adapter-ligated library. In other embodiments, the amplification mixture includes a Library Amplification Hot Start Master Mix and a xGen UDI primer Mix (IDT). In some embodiments, the amplification mixture comprises a total volume ranging from 10 to 100 μl.

In some embodiments, the method comprises amplifying the amplification mixture to produce a first set of amplicon products. In some embodiments, amplifying is performed using a thermocycler. In some embodiments, amplifying is performed using polymerase chain reaction (PCR). In some embodiments, amplifying comprises running the amplification mixture in the thermocycler for a duration ranging from 1 second to 5 minutes. In some embodiments, the temperature of incubation of the amplification mixture in the thermocycler ranges from 4° C. to 110° C.

In some embodiments, the resulting precursor libraries containing the nucleic acid fragments comprise a 5′ consensus read region; a 3′ consensus read region; and a target region.

In some embodiments, (i) the 5′ consensus read region is a read1 sequence or a reverse complement thereof and the 3′ consensus read region is a read2 sequence or a reverse complement thereof or (ii) the 5′ consensus read region is a read2 sequence or a reverse complement thereof and the 3′ consensus read region is a read1 sequence or a reverse complement thereof.

### Amplification Based Based Library Preparation

As an alternative to ligation-based library preparation of precursor libraries, the methods of the present disclosure can instead use an amplicon-based library preparation method. In the amplicon-based library preparation method, the method includes amplifying, in each cell within the cell, nucleic acids (e.g. DNA or RNA) with a primer pool set to produce a first set of amplicon products for each cell.

In some embodiments, the primers in the primer pool set are DNA primers. In some embodiments, the primers in the primer pool set are RNA primers. In some embodiments, the primer pool set includes targeting primers for targeting the target sequence region of the DNA or RNA within the cell.

Primers may include modifications such as: methylation, capping, 3′-deoxy-2′,5′-DNA, N3′ P5′ phosphoramidates, 2′-O-alkyl-substituted DNA, 2′-O-methyl DNA, 2′ Fluoro DNA, Locked Nucleic Acids (LNAs) with 2′-O-4′-C methylene bridge, inverted T modifications (e.g. 5′ and 3′), or PNA (with such modifications at one or more nucleotide positions). Ligation adapter sequences may also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters).

### Primer Sets

In some embodiments, the first primer pool set of the present disclosure is designed to amplify multiple targets with the use of multiple primer pairs in a PCR experiment (e.g. in 1 or more PCR steps, 2 or more PCR steps, or 3 or more PCR steps).

In some embodiments, specific target sites are selected (particularly during the amplicon-based library preparation). In some embodiments, 1-10 target loci are selected.

In some embodiments the first primer pool set comprises a first forward primer pool. In some embodiments, the first primer pool set comprises a first reverse primer pool. The number of primers within each primer pool set is dependent on the number of targets that will be prepared using the amplicon-based method. In some embodiments, the primers in the primer pool set further comprises indexing primers (e.g. barcoding primers).

In some embodiments the primer pool set comprises a first forward primer pool and a reverse primer pool. In some embodiments, each forward primer and each reverse primer includes a nucleotide sequence having a length ranging from 10 to 200 nucleotides. In some embodiments, each forward and each reverse primer includes a nucleotide sequence having a length of 10, 11, 12, 13, 14, 15, 16, 17, 18, 19, 20, 21, 22, 23, 24, 25, 26, 27, 28, 29, or 30 nucleotides. Forward primers within the set of forward primers can have different lengths. Similarly, reverse primers within the set of reverse primers can have different lengths. In certain embodiments, forward primers within the set of forward primers can have different lengths but similar Melting Temperature (Tm) and thus can have similar PCR reaction times. Reverse primers within the set of reverse primers can have different lengths but similar Melting Temperature (Tm) and thus can have similar PCR reaction times.

In some embodiments, each forward primer comprises a nucleotide sequence that hybridize to an anti-sense strand of a nucleotide sequence encoding a target region (e.g., target region of the DNA or RNA) of one or more cells. In some embodiments, the nucleotide sequence is a DNA sequence. In some embodiments, the nucleotide sequence is an RNA sequence. In some embodiments, each primer comprises a unique nucleotide sequence that hybridizes to an anti-sense strand of a nucleotide sequence encoding a different target region (e.g., a different target region of the DNA or RNA) of one or more cells. Thus, a forward primer pool can include a plurality of forward primers, where each forward primer hybridizes to a distinct target nucleic acid.

In some embodiments, each reverse primer comprises a nucleotide sequence that hybridize to a sense strand of a nucleotide sequence encoding a target region of one or more cells. In some embodiments, each primer comprises a unique nucleotide sequence that hybridizes to an anti-sense strand of a nucleotide sequence encoding a different target region of one or more cells. Thus, a reverse primer pool can include a plurality of reverse primers, where each reverse primer hybridizes to a distinct target nucleic acid. In some embodiments, the primers can include a modification that is cleaved off before they are able to polymerize.

As described herein, a first primer pool set can include publicly available primer pool sets of known nucleic target regions of interest. In some embodiments, the first primer pool set can include any standard multiplexing primer panel for sequencing. In some embodiments, a forward primer pool includes primers selected from a rhAmp PCR Panel, CleanPlex® NGS Panel, and Ampliseq Panel. In some embodiments, a reverse primer pool includes primers of a rhAmp PCR Panel, CleanPlex® NGS Panel, and Ampliseq Panel. However, the forward and revers primers do not need to be from any existing panels. In some embodiments, the primer pool set comprises RNA: DNA hybrids. In some embodiments the panel includes only the target regions of interest. In some embodiments the panel includes both the target region of interest and a common sequence, such that the target region of interest is on the 3′ end of the common sequence.

Aspects of the present disclosure include amplifying the DNA or RNA within the cell/nuclei population using the first primer pool set to produce a first set of amplicon products. In some embodiments, the nucleic acids of the cell are amplified in situ.

The term “amplicon”, as used herein and in its conventional sense, refers to the amplified nucleic acid product of a PCR reaction or other nucleic acid amplification process (e.g., ligase chain reaction (LGR), nucleic acid sequence-based amplification (NASBA), transcription-mediated amplification (TMA), Q-beta amplification, strand displacement amplification, target mediated amplification, and the like). Amplicons may comprise RNA or DNA depending on the technique used for amplification. For example, DNA amplicons may be generated by RT-PCR, whereas RNA amplicons may be generated by TMA/NASBA.

### Multiplexed Polymerase Chain Reaction

As explained above, the primer sets described herein by are used in in situ PCR for amplification of target nucleic acids in a sample containing cells. PCR is a technique for amplifying desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

The present methods can use PCR for amplification of DNA or RNA fragments in one or more PCR reactions, with one or more of the PCR steps occurring in situ. As a non-limiting example, in a multiplexing assay, more than one target sequence can be amplified by using multiple primer pairs in a reaction mixture. PCR steps can also be used to create copies of amplicon products containing the DNA or RNA products. In some embodiments, multiple PCR reactions are performed between the first amplification step (e.g., target amplification) and the sequencing steps.

In particular, PCR uses relatively short oligonucleotide primers which flank the target nucleotide sequence to be amplified, oriented such that their 3′ ends face each other, each primer extending toward the other. The polynucleotide sample is extracted and denatured, e.g., by heat, and hybridized with first and second primers that are present in molar excess. Polymerization is catalyzed in the presence of the four deoxyribonucleotide triphosphates (dNTPs--dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. In some cases, PCR is carried out with a commercially available thermal cycler, e.g., Perkin Elmer, ProFlex PCR system, VeritiPro Thermal Cycler, Automated Thermal Cycler, SimpliAmp Thermal Cycler, MiniAmp thermal Cycler, C100 Touch Thermal Cycler, S1000 Thermal cycler, or T100 Thermal Cycler.

RNA may be amplified by reverse transcribing the RNA into cDNA (RT-PCR) using an RNA dependent DNA polymerase (RT-PCR) with a single targeting primer set to the anti-sense strand of RNA, oligo-dT primers, or random sequences, such as a random hexamer. PCR amplification can then occur with addition targeting primers as described above. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall et al. (1994) PCR Meth. App. 4:80-84. Suitable DNA polymerases include reverse transcriptases, such as avian myeloblastosis virus (AMV) reverse transcriptase (available from, e.g., Seikagaku America, Inc.), Moloney murine leukemia virus (MMLV) reverse transcriptase (available from, e.g., Bethesda Research Laboratories), HIV reverse transcriptase, and Telomerase reverse transcriptase.

Any PCR reaction mixture (e.g., used interchangeably herein as “PCR Enzyme Master Mix”) and heat-resistant DNA polymerase may be used to produce amplicon products. For example, those contained in a commercially available PCR kit can be used. In some embodiments, the PCR reaction mixture can include other enzymes that aid in transcription (e.g., such as RNAseH to cleave a modification in primers). Non-limiting examples of a PCR kit includes rhAmpSeq Library Kit (IDT) and rhAmpSeq Library Mix. In some embodiments, one or more components of a PCR kit can be used in the PCR reaction mixture, at various concentrations. As the reaction mixture, any buffer known to be usually used for PCR can be used. Examples include IDTE (10 mM Tris, 0.1 mM EDTA; Integrated DNA Technologies), Tris-HCl buffer, a Tris-sulfuric acid buffer, a tricine buffer, and the like. Examples of heat-resistant polymerases include Taq DNA polymerase (e.g., FastStart Taq DNA Polymerase (Roche), Ex Taq (registered trademark) (Takara), Z-Taq, AccuPrime Taq DNA Polymerase, M-PCR kit (QIAGEN), KOD DNA polymerase, Pfu DNA polymerase, and the like.

In some embodiments, the heat resistant DNA polymerase has a low error rate and has a high degree of accuracy for DNA replication. In some embodiments, the heat resistant DNA polymerase is a high-fidelity polymerase (Hi-Fi). Examples of Hi-Fi DNA polymerases include, but are not limited to, Phusion High-Fidelity DNA Polymerase, Phusion Plus DNA polymerase, VWR® HiFi DNA polymerase, ALLinTM HiFi DNA polymerase, and AccuPrime Taq DNA Polymerase. In some embodiments, the heat resistant DNA polymerase is modified so that it is unreactive at ambient temperatures. This allows for a reduction of non-specific amplification. In some embodiments, the heat resistant DNA polymerase is a hot-start DNA polymerase. Examples of hot-start DNA polymerases include, but are not limited to, DreamTaq Hot Start DNA polymerase, Takara Taq DNA polymerase, and KOD Hot Start DNA polymerase. In some embodiments, the heat resistant DNA polymerase is capable of amplifying long DNA strands. This could be DNA polymerases that are capable of amplifying fragments of up to 30 Kb in length. In some embodiments, the heat resistant DNA polymerase is a long-range DNA polymerase. Examples of long-range DNA polymerases include, but are not limited to, LA Taq DNA polymerase, QIAGEN LongRange PCR kit and Platinum SuperFi II DNA Polymerase.

The amounts of the primer and template DNA used, etc., in the present disclosure can be adjusted according to the PCR kit and device used. In some embodiments, about 0.1 to 1 μl of the first primer pool set is added to the in situ PCR reaction mixture.

In some embodiments, the PCR reaction mixture includes the first primer pool set, the population of cells, and a PCR library mix. Any standard PCR library mix can be used in the PCR reaction mixture. In some embodiments, the library mix is a rhAmpSeq Library Mix or components of the rhAmpSeq Library Mix. In some embodiments, the PCR library mix contains one or more components of a rhAmpSeq Library mix or one or more components of any standard PCR Library mixture. In some embodiments, a forward primer pool of the first primer pool set includes forward primers of a rhAmp PCR Panel. In some embodiments, a reverse primer pool of the first primer pool set includes reverse primers of a rhAmp PCR Panel. However, any standard PCR library mix or PCR Enzyme Master Mix for sequencing can be used.

In some embodiments, about 0.1 to 10 μl of the PCR library mix is added to the PCR reaction mixture. The PCR reaction mixture of the present disclosure includes one or more cell populations. In some embodiments, the cell population is diluted to a volume of about 0.5 μl to about 20 μl.

As described herein, the PCR cycling conditions are not particularly limited as long as the desired target genes can be amplified. For example, the thermal denaturation temperature can be set to 92 to 100° C., e.g., 94 to 98° C. The thermal denaturation time can be set to, for example, 5 to 180 seconds, e.g., 10 to 130 seconds. The annealing temperature for hybridizing primers can be set to, for example, 55 to 80° C., e.g., 60 to 70° C. The annealing time can be set to, for example, 10 to 60 seconds, e.g., 10 to 20 seconds. The extension reaction temperature can be set to, for example, 55 to 80° C., e.g., 60 to 70° C. The elongation reaction time can be set to, for example, 4 to 15 minutes, e.g., 10 to 20 minutes. In some embodiments, the annealing and extension reaction can be performed under the same conditions. In some embodiments, the operation of combining thermal denaturation, annealing, and an elongation reaction is defined as one cycle. This cycle can be repeated until the required amounts of amplification products are obtained. For example, the number of cycles can be set to 30 to 40 times, e.g., about 30 to 35 times. In some embodiments, the number of cycles can be set to 5 to 10 cycles, 10 to 15 cycles, 15 to 20 cycles, 20 to 25 cycles, 25 to 30 cycles, 35 to 40 cycles, 45 to 50 cycles, or 55 to 60 cycles.

In some embodiments, the optimal amount for in-solution PCR (1X) of polymerase enzyme is also used in the in situ PCR reaction. For Taq polymerase an optimal amount of polymerase for in-solution PCR may be from about 0.25 units/50 μl to about 2.5 units/50 μl where one unit is defined as the amount of enzyme needed to catalyze the incorporation of 10 nanomoles of deoxyribonucleotides into acid-insoluble material in 30 minutes at 70° C. using herring sperm DNA as a substrate. For TaKaRa Ex Taq polymerase an optimal amount of polymerase for in-solution PCR may be from about 2.5 units/50 μl to about 5 units/50 μl where one unit is defined as the amount of enzyme that will incorporate 10 nmol of dNTP into acid-insoluble products in 30 minutes at 74° C. with activated salmon sperm DNA as the template-primer. For AccuPrime Taq polymerase an optimal amount of polymerase for in-solution PCR may be from about 1 units/50 μl to about 2.5 units/50 μl where one unit is defined as the amount of enzyme which incorporates 10 nmol of deoxyribonucleotide into DNA in 30 minutes at 74° C. For Pfu DNA polymerase an optimal amount of polymerase for in-solution PCR may be from about 2.5 units/50 μl to about 5 units/50 μl where one unit is defined as the amount of enzyme required to catalyze the incorporation of 10 nmol of dNTPs into acid-insoluble material in 30 minutes at 75° C.

In some embodiments, an increased amount of enzyme is used for the in situ PCR reaction. In some embodiments, 2X, 3X, 4X, 5X, 6X, 7X, 8X, 9X, or 10X the concentration of enzyme is used for the in situ PCR reaction.

In some embodiments, an appropriate concentration of forward and reverse primers are used in the PCR reaction. An appropriate concentration of primers may be from about 0.1 μM to about 1 μM. In some embodiments, a concentration of primer greater than 1 μM is used in the PCR reaction.

In the present disclosure, the “PCR cycling conditions” may include one of, any combination of, or all of the conditions with respect to the temperature and time of each thermal denaturation, annealing, and elongation reaction of PCR and the number of cycles. When PCR cycling conditions are set, the touchdown PCR method can be used in terms of inhibiting non-specific amplification. Touchdown PCR is a technique in which the first annealing temperature is set to a relatively high temperature and the annealing temperature is gradually reduced for each cycle, and, midway and thereafter, PCR is performed in the same manner as general PCR. Shuttle PCR may also be used in terms of inhibiting non-specific amplification. Shuttle PCR is a PCR in which annealing and extension reaction are performed at the same temperature. Nested PCR may also be used for inhibiting non-specific amplification. Nested PCR is when PCR is done with two sets of primers, an inner and outer set. The outer primers are used to generate the DNA products first, followed by the inner primers. The likelihood of the outer primers amplifying the wrong locus followed by the inner primers also amplifying this locus is very small. Multiplex-PCR can be used to amplify multiple targets in a single PCR experiment. This works by using multiple primer sets which have been optimized to work simultaneously in a single reaction.

Although different PCR cycling conditions can be used for each primer pair, it is preferable from the viewpoint of operation and efficiency that PCR cycling conditions are set in such a manner that the same PCR cycling conditions can be used for different primer pairs and the variation of PCR cycling conditions used to obtain necessary amplification products is minimized. The number of variations of PCR cycling conditions is preferably 10 or less, 5 or less, more preferably 4 or less, still more preferably 3 or less, even more preferably 2 or less, and even still more preferably 1. When the number of variations of PCR cycling conditions used to obtain all the necessary amplification products is reduced, PCRs using the same PCR cycling conditions can be simultaneously performed using one PCR device. Accordingly, the desired amplification products can be obtained in a short time using smaller amounts of resources.

In some embodiments, the method of the present disclosure includes, after producing the first set of amplicon products, purifying the first set of amplicon products. Techniques for purifying amplicon products are well-known in the art and include, for example, using magnetic bead purification reagent, passing through a column, use of ampure beads, phenol chloroform and the like.

Other non-limiting examples of purifying amplicons include, using size selection based magnetic bead purification reagent (e.g., Solid Phase Reversible Immobilization (SPRI) beads), passing through a column, phenol chloroform and the like. In some embodiments, purifying the ligated DNA or RNA fragments can include using magnetic streptavidin beads, for example if the DNA or RNA fragments contain biotin. In some embodiments, the bead purification method uses Solid Phase Reversible Immobilisation (SPRI) beads. In some embodiments, the purification beads are made from polysterene-magnetite. These beads can be coated with negatively charged carboxyl groups. Bead-based size purification can include a step which involves the addition of an appropriate amount of salt (Na+) to aid in the precipitation of the DNA/RNA. The bead-based purification method can also include a size selection step. The bead-based purification method can also include an elution step through the addition of an aqueous solution. Examples of aqueous solutions for elution include, but are not limited to, water, nuclease free water, and Tris-EDTA. In some embodiments, the beads are magnetic beads. These beads can bind to DNA/RNA in a pH dependent manner. The magnetic beads may be positively charged at low pH, and negatively charged at high pH. The pH of the DNA/RNA sample may be controlled to allow the DNA/RNA binding to beads or its release from the beads. In some embodiments, the column based purification is silica based. This may require the presence of chaotropic salts. An non-limiting example of a chaotropic salt is guanidine hydrochloride. The chaotropic salt may be present in high quantities. The column based purification may involve one or more wash steps with an appropriate buffer. Examples of appropriate buffers include, but are not limited to, salt and/or ethanol solutions. The DNA/RNA can then be eluted in an appropriate elution buffer. Examples of appropriate elution buffers include, but are not limited to, water, nuclease free water, and Tris-EDTA. The DNA/RNA would usually be eluted under low salt conditions. In some embodiments, the phenol-chloroform purification method involves adding the phenol-chlorform mixture to equal volume of the DNA/RNA sample. Phenol-chloroform purification involves the extraction of DNA/RNA through isolation of the aqueous phase. The phenol-chloroform purification procedure can be repeated one or more times to increase the purity of the DNA/RNA. In some embodiments, the phenol: chloroform ratio in the phenol-chloroform mixture is made close to a 1:1 ratio. In some embodiments, the phenol-chloroform mixture also contains alcohol. An non-limiting example of an alcohol which can be used is isomyl alcohol. An appropriate amount of isomyl alcohol is added to the phenol-chloroform mixture. The phenol: chloroform: isomyl alcohol ratio can be approximately 25:24:1. In some embodiments, the phenol-chloroform mixture is buffered. The phenol-chlorform purification method may include an additional ethanol precipitation step. The ethanol precipitation step involves isolating the DNA/RNA in a precipitate.

### Additional Exemplary Amplification or Ligation Steps

The amplicon-based method of the present disclosure can include multiple additional PCR steps after the first amplification step and before sequencing. For example, additional PCR steps can be performed before or after lysing or after purification. The method can also include ligation steps to ligate on adapter sequences for subsequent PCR or direct sequencing.

Adapter sequences may include modifications such as: methylation, capping, 3′-deoxy-2′,5′-DNA, N3′ P5′ phosphoramidates, 2′-O-alkyl-substituted DNA, 2′-O-methyl DNA, 2′ Fluoro DNA, Locked Nucleic Acids (LNAs) with 2′-O-4′-C methylene bridge, inverted T modifications (e.g. 5′ and 3′), or PNA (with such modifications at one or more nucleotide positions). Ligation adapter sequences may also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters).

In some embodiments, the method further comprises amplifying the first set of amplicon products with primer sequences to produce a set of amplicon products. This step can be performed after the first amplification step and before the lysing step, after the lysing step, or after a second amplification step (e.g., amplification with sample barcoding sequences). In some embodiments, the primer sequences include sample barcodes.

In some embodiments, the method further comprises, after the sorting step or lysing step, contacting the first set of amplicon products with sample barcoding sequences. In some embodiments, sample barcoding sequences comprise a set of forward and/or reverse sample barcoding primers, and wherein the method comprises amplifying the first set of amplicon products with the set of forward and/or reverse sample barcoding primers to produce a barcoded indexed library comprising sample barcoded amplicon products.

As an alternative to amplification of sample barcoding sequences, in some embodiments, the sample barcoding sequences comprise a set of barcoding adapters, and wherein the method comprises ligating the set of barcode adapters to produce a barcoded indexed library comprising barcoded amplicon products.

In some embodiments, the method further comprises ligating on adapter sequences. Non-limiting examples of adapter sequences include, but are not limited to, adapter nucleotide sequences that allow high-throughput sequencing of amplified nucleic acids. In some embodiments, the adapter sequences are selected from one or more of: a Y-adapter nucleotide sequence, a hairpin nucleotide sequence, a duplex nucleotide sequence, and the like. In some embodiments, the adapter sequences are for pair-end sequencing. In some embodiments, the adapter sequences include sequencing reads (e.g., R1, R2, etc.). In some embodiments, the adapter sequences include sample barcodes. Adapter sequences can be used in a ligation reaction of the disclosed method for the desired sequencing method used.

Barcodes used in amplicon-based library preparation methods can have the same characteristics as described above for ligation-based library preparation e.g. the use of degenerate sequences, etc.

In some embodiments, ligating includes performing ligase chain reaction (LCR). The ligase chain reaction (LCR) is an amplification process that involves a thermostable ligase to join two probes or other molecules together. In some embodiments, the ligated product is then amplified to produce a second amplicon product. In some embodiments, LCR can be used as an alternative approach to PCR. In other embodiments, PCR can be performed after LCR.

In some embodiments, the thermostable ligase can include, but is not limited to, Pfu ligase, Taq ligase, HiFi Taq DNA ligase, 9°N DNA ligase, Thermostable 5′ AppDNA/RNA ligase, Ampligase® ligase, or a T4 RNA ligase (e.g. T4 RNA ligase 2). In some embodiments, the method further comprises, after the sorting step or lysing step, contacting the first set of amplicon products with sample barcoding sequences. In some embodiments, sample barcoding sequences comprise a set of forward and/or reverse sample barcoding primers, and wherein the method comprises amplifying the first set of amplicon products with the set of forward and/or reverse sample barcoding primers to produce a barcoded indexed library comprising sample barcoded amplicon products.

In some embodiments, at least two in situ PCRs are done for amplicon-based library preparation. The first PCR is used to produce a first set of amplicons, and the second PCR is done after the first PCR to amplify from the first set of amplicons. In some embodiments, the second PCR step also adds barcoding and/or adapter sequences to the first set of amplicons.

### Mixing Cells Containing Precursor Libraries

In some embodiments of the present method, once precursor libraries are prepared in situ within the cells, the method comprises preparing a mixture, in one or more containers, comprising one or more of: the cell population, cell barcoding oligonucleotides, and amplification reagents.

In some embodiments, the method comprises mixing the cells containing the precursor libraries with cell barcoding oligonucleotides and amplification reagents in a single container. In some embodiments, the method comprises mixing the cells containing the precursor libraries with cell barcoding oligonucleotides in a single container. In some embodiments, the method comprises mixing the cells containing the precursor libraries with amplification reagents in a single container. In some embodiments, the method comprises mixing the cells containing the precursor libraries with amplification reagents in a single container. In some embodiments, the cell barcoding oligonucleotides are in a separate container from the precursor libraries.

### Partitioning the Mixture

Once cells containing precursor library are prepared the method includes partitioning the mixture into a plurality of partitions.

In some embodiments, the partitions contain: a cell from the cell population comprising the precursor library, and a plurality of cell barcoding oligonucleotides. In some embodiments, the partitions comprise a cell from the cell population.

In some embodiments methods of partitioning can include loading a single mixture containing the cells, barcodes and amplification reagents into a partitioning engine. In some embodiments, partitioning comprises loading the cells containing precursor libraries into the partitioning engine. In some embodiments, partitioning comprises loading the cells containing precursor libraries, barcodes, and amplification reagents into the partitioning engine with one or more mixtures.

In some embodiments, the partitioning engine comprises a microfluidic chip and a partitioning engine. In some embodiments, the cells are loaded into the microfluidic chip. In some embodiments, the microfluidic chip contains an oil surface. An example of a partitioning method is described in “Protocol K” of Example 3. Commercially available instruments can be used to partition the mixture into a plurality of partitions, for example, a Geode (Stilla Technologies) instrument, a droplet generator, digital PCR, and the like. In some embodiments, For example, if the partition is a droplet, in some embodiments, droplets will form around a cell with amplification enzymes and barcoding oligonucleotides as they contact an oil layer of the microfluidic chip.

Single cell, digital PCR and cell sorting instruments that can be used for performing mixing, partitioning, cellular barcoding steps, and optionally cell sorting, of the present methods include, but are not limited to: Chromium (10X Genomics), ddSEQ Single-Cell Isolator (Biorad), QX200 Droplet Digital PCR System (Biorad), QX600 Droplet Digital PCR System (Biorad), QX One Droplet Digital PCR System (Biorad), Qiacuity Digital PCR Machine (Qiagen), QuantStudio Absolute Q Digital PCR System (ThermoFisher), HIVE CLX (Honeycomb), BD Rhapsody (BD Biosciences), Icell8 (Takara), Asteria (Scipio), Pipseq (Fluent), Biomark X9 System (Standard Biotools), C1 System (Standard Biotools), SH800S Cell Sorter (Sony Biotechnology), BD FACS Melody (BD Biosciences), CytoFlex SRT Benchtop Cell Sorter (Beckman Coulter), and MoFlo Astrios Cell Sorter (Beckman Coulter).

### Concentration of Barcoding Oligonucleotides in Each Partition

In some embodiments, the concentration of barcoding oligonucleotides in the at least some partitions ranges from 25 to 600 pM. In some embodiments, the concentration of barcoding oligonucleotides in the at least some partitions ranges from 45 pM to 550 pM.

In some embodiments, the concentration of barcoding oligonucleotides in the at least some partitions ranges from 50 nM to 500 nM. In some embodiments, the concentration of barcoding oligonucleotides comprises 500 nM. In some embodiments, the concentration of barcoding oligonucleotides comprises 50 nM.

In some embodiments, the concentration of barcoding oligonucleotides comprises 5 pM to 500 pM.

In some embodiments, the amount of barcoding oligonucleotides in each partition containing the cell ranges from 50 barcoding oligonucleotides to 10 million barcoding oligonucleotides (e.g., such as at least 50, at least 100, at least 150, at least 200, at least 250, at least 300, at least 350, at least 400, at least 450, at least 500, at least 1000, at least 2000, at least 5000, at least 10,000, at least 20,000, at least 50,000, at least 100,000 at least 200,000, at least 500,000, or at at least 1,000,000 barcoding oligonucleotides). In some embodiments, the number of barcoding oligonucleotides is at least 80. In some embodiments, the number of barcoding oligonucleotides is at least 800. In some embodiments, the number of barcoding oligonucleotides is at least 8000. In some embodiments, the number of barcoding oligonucleotides is at least 80,000. In some embodiments, the number of barcoding oligonucleotides is at least 800,000. In some embodiments, the number of barcoding oligonucleotides is at least 8,000,000. Table 1 below provides an exemplary table of amount of barcoding oligonucleotides needed per cell:

TABLE 1
Barcoding Oligonucleotides Scheme

| | | # Barcodes |
|---|---|---|
| | Cell Molecules | Per |
| | Present in 1 ul of | Container |
| Concentration | Stock | (partition) |

| | | | |
|---|---|---|---|
| 500 | nM | 3.01E+11 | 8,189,920 |
| 50 | nM | 3.01E+10 | 818,992 |
| 5 | nM | 3.01E+09 | 81,899 |
| 500 | pM | 3.01E+08 | 8,190 |
| 50 | pM | 3.01E+07 | 819 |
| 5 | pM | 3.01E+06 | 82 |

In some embodiments, each partition in the plurality of partitions holds a volume ranging from 0.1 to 5 nanoliters.

In some embodiments, each partition in the plurality of partitions has a volume ranging from 0.1 to 1 nanoliters.

In some embodiments, the at least some partitions comprise at least 80% partitions containing cells from the cell population comprising the precursor library and the plurality of cell barcoding oligonucleotides.

In some embodiments, the at least some partitions comprise at least 70% partitions containing cells from the cell population comprising the precursor library and the plurality of cell barcoding oligonucleotides.

In some embodiments, the at least some partitions comprise at least 60% partitions containing cells from the cell population comprising the precursor library and the plurality of cell barcoding oligonucleotides.

In some embodiments, the at least some partitions comprise at least 50% partitions containing cells from the cell population comprising the precursor library and the plurality of cell barcoding oligonucleotides.

In some embodiments, the at least some partitions comprise at least 40% partitions containing cells from the cell population comprising the precursor library and the plurality of cell barcoding oligonucleotides.

In some embodiments, the at least some partitions comprise at least 30% partitions containing cells from the cell population comprising the precursor library and the plurality of cell barcoding oligonucleotides.

In some embodiments, the at least some partitions comprise at least 20% partitions containing cells from the cell population comprising the precursor library and the plurality of cell barcoding oligonucleotides.

In some embodiments, the at least some partitions comprise at least 10% partitions containing cells from the cell population comprising the precursor library and the plurality of cell barcoding oligonucleotides.

In some embodiments, the at least some partitions comprise at least 5% partitions containing cells from the cell population comprising the precursor library and the plurality of cell barcoding oligonucleotides.

In some embodiments, cells that are not in partitions are also going through the remainder of steps within the methods of the present disclosure, such as cellular barcoding, and isolation of the cell barcoded library.

### NON-LIMITING EXEMPLARY EMBODIMENTS OF PARTITIONING

### Embodiment 1. In Situ Library Prep Using Combinatorial Cell Barcoding

In some cases, problems of low cell throughput in the Double Poisson method (Option 2). This disclosure features a method that has been developed to facilitate cell identification when multiple cell barcodes enter a partition (e.g., a droplet prepared by the digital PCR instrument). Providing cell barcode primers to both universal sequences in the library creates a bipartite graph of cell barcode combinations.

In this embodiment, the following reagents are distributed to the partitions, such that some partitions, but not necessarily all partitions, include 1) in situ library prepped cells (precursor libraries), 2) entities containing cell barcode primers designed UniversalSequence1, 3) entities containing cell barcode primer designed to UniversalSequence2, 4) enzymes, and 5) optimal buffers. In some instances, the reagents are mixed together (in some cases) before partitioning by preparing a single master mix of the items, or multiple master mixes (each including at least one of the items) are combined on the instrument (e.g., the dPCR instrument) or in the partition.

In some embodiments, partition components include one or more of:
1) Cells provided in a concentration such that the mean number of cells in a partition is less than 0.5, less than 1, less than 2, less than 5, or less than 10.
2) Entities containing cell barcode primers having complementarity to Universal Sequencel are provided in a concentration such that the mean number of entities containing cell barcode primers in a partition is greater than 0.5, greater than 1, greater than 2, greater than 5, greater than 10, or greater than 100, greater than 1000, greater than 10000, greater than 100000, greater than 200,000, greater than 500,000, greater than 1,000,000, greater than 2,000,000, greater than 3,000,000, greater than 5,000,000, greater than 7,000,000, greater than 8,000,000, or greater than 9,000,000. In some instances, entities can be a droplet, bead, or product of rolling circle amplification, such that multiple copies of the cell barcode primer will be provided with one entity. Processing of the barcode primer in the partition can be done as needed, such as a user digestion to cleave primers from the bead. Entities are provided such that a unique cell barcode sequence is present less than 10, less than 5, or less than 1 time within the entire reaction.

The cell barcode primers amplify the in situ prepared library by hybridizing to the complement of one of the universal sequences.

In some embodiments, amplifying the cell barcode primers produce a molecule that can amplify the precursor library and have compositions including: 5′-Additional Universal Sequence-Cell Barcode-Universal Sequence 1-3′.

3. Entities containing cell barcode primers to greater than 0.5, greater than 1, greater than 2, greater than 5, greater than 10, or greater than 100, greater than 1000, greater than 10000, greater than 100000, greater than 200,000, greater than 500,000, greater than 1,000,000, greater than 2,000,000, greater than 3,000,000, greater than 5,000,000, greater than 7,000,000, greater than 8,000,000, or greater than 9,000,000. In some instance, entities can be a droplet, bead, or product of rolling circle amplification, such that multiple copies of the cell barcode primer will be provided with one entity. Processing of the barcode primer in the partition can be done as needed, such as a User digestion to cleave primers from the bead. Entities are provided such that a unique cell barcode sequence is present less than 10, less than 5, or less than 1 time within the entire reaction.

The cell barcode primers amplify the in situ prepared library by hybridizing to the complement of one of the universal sequences.

In some embodiments, amplifying the cell barcode primers produce a molecule that can amplify the precursor library and have compositions including: 5′-Additional Universal Sequence-Cell Barcode-Universal Sequence 2-3′.

4. Enzymes: In some instances, the partition includes at least an amplification enzyme for amplification with the provided primers. In some instances, a lysis enzyme is provided to lyse the cells in the partition. In some instances a cleavage enzyme to release barcode primers is included. In some instances, inclusion or exclusion of enzymes depends, at least in part on, on whether the enzyme is rate limiting to the reaction in each partition.

5. Buffers: In some instances, a buffer can be any appropriate buffer. In some instance, a buffer is a buffer that enables all reactions to be performed. In some instances, activity of any or all of the enzymes can be 100%, 90%, 80%, 70%, 50%, 25%, 10% of optimal enzyme activity in the buffer.

In some instances, barcode deconvolution is performed according to the following:
Each partition should get one or more entities containing cell barcode primers to UniversalSequence1 and one or more entities containing cell barcode primers to UniversalSequence2, however, due to statistical representation of unique cell barcode primers in the reaction, each cell should have a unique combination of cell barcode primers. In the ideal case the barcode primer combinations should be non-overlapping (i.e., every partition only contains sequences that are not present in any other partition), however, computational methods can be used to deconvolute some overlap in barcode primer representation.

A bipartite graph linking the cell barcodes attached to UniversalSequence1 and the cell barcodes attached to UniversalSequence2 can be made from the sequencing data. Linking reads with different Cell barcode sequences together via the bipartite graph.

In some embodiments, the in-partition amplification is used performed in a digital PCR instrument using digital PCR workflow or digital PCR workflow adapted to include the described steps.

### Embodiment 2. In Situ Library Prep Using Cell Barcoding

This disclosure features a method that is described as double Poisson cell sorting because it relies on the Poisson distribution of both the cells and the cell barcode primers to enable identification of unique cells. In some instances, barcode merging, according to existing methods known in the art, can be used to improve throughput.

In this embodiment, the following items are distributed to the partitions (e.g., a droplet prepared by the digital PCR instrument), such that some partitions, but not necessarily all partitions, will include 1) in situ library prepped cells, 2) cell barcode primers, 3) sample barcode primers, 4) enzymes, and 5) optimal buffers. In some instances, the reagents are mixed together before partitioning by preparing a single master mix of the items, or multiple master mixes (each including at least one of the items) are combined on the instrument (e.g., the dPCR instrument) or in the partition.

In some instances, after partitioning the reagents, temperature cycling is performed to allow for all enzymatic reactions to take place. Libraries are then recovered from the partitions. Additional amplification is optional. Sequencing of the recovered libraries is performed. Notably, unlike digitalPCR, libraries produced with this method will contain slightly different sequences between partitions, specifically at the cell barcode position.

In some embodiments, partition components include one or more of:
1) Cells provided in a concentration such that the mean number of cells in a partition is less than 0.5, less than 1, less than 2, less than 5, or less than 10.
2) Entities containing cell barcode primers having complementarity to one of the UniversalSequences are provided in a concentration such that the mean number of entities containing cell barcode primers in a partition is less than 0.5, less than 1, less than 2, less than 5, or less than 10. In some instances, entities can be a droplet, bead, or product of rolling circle amplification, such that multiple copies of the cell barcode primer will be provided with one entity. Processing of the cell barcode primer in the partition can be done as needed, such as a User digestion to cleave primers from the bead. Entities are provided such that a unique cell barcode sequence is present less than 10, less than 5, or less than 1 time within the entire reaction.

The cell barcode primers amplify the in situ prepared library by hybridizing to the complement of one of the universal sequences.

In some embodiments, amplifying the cell barcode primers produce a molecule that can amplify the precursor library and have including: 5′-Additional Universal Sequence-Cell Barcode-Universal Sequence 1-3′ or 5′-Additional Universal Sequence-Cell Barcode-Universal Sequence 2-3′

3. In some embodiments, sample barcode primers are provided to amplify the in situ prepared library. The sample barcode primers hybridize to the complement of the other universal sequence to facilitate amplification. In such instances, the partition can contain a sample barcode and/or other additional universal sequences. In some instances, the sample barcode is provided at a concentration such that enough primers are present in each partition, for example, greater than 10, greater than 100, greater than 1000, greater than 5000, or greater than 10,000 copies per partition.

In some embodiments, amplifying the sample barcode primers produce a molecule that can amplify the precursor library and have compositions including: 5′-Additional Universal Sequence-Sample Barcode-Universal Sequence 1-3′; 5′-Additional Universal Sequence-Sample Barcode-Universal Sequence 2-3′; 5′-Additional Universal Sequence-Universal Sequence 1-3′; 5′-Additional Universal Sequence-Universal Sequence 2-3′; 5′-Universal Sequence 1-3′; and 5′-Universal Sequence 2-3′.

4. In some instances, the partition includes at least an amplification enzyme for amplification with the provided primers. In some instances, a lysis enzyme is provided to lyse the cells in the partition. In some instances, a cleavage enzyme to release barcode primers is also included. In some instances, inclusion or exclusion of enzymes depends, at least in part on, on whether the enzyme is rate limiting to the reaction in each partition.

5. Buffers: In some instances, a buffer can be any appropriate buffer. In some instance, a buffer is a buffer that enables all reactions to be performed. In some instances, activity of any or all of the enzymes can be 100%, 90%, 80%, 70%, 50%, 25%, 10% of optimal enzyme activity in the buffer.

In some embodiments, the in-partition amplification is used performed in a digital PCR instrument using digital PCR workflow or digital PCR workflow adapted to include the described steps.

### Performing Cell Barcoding of Precursor Libraries, in Partitions

An aspect of the present method includes barcoding the precursor libraries in the plurality of partitions by amplifying the cell barcoding oligonucleotides in the at least some partitions to produce cell barcoding primers; and amplifying the precursor libraries in partitions with the cell barcoding primers to produce cell barcoded libraries.

General methods of cell barcoding of precursor libraries can be found in PCT Application Publication No.: WO2022/192603, and U.S. application Ser. No. 18/467,315, which are hereby incorporated by reference in their entireties. For example, in the process of performing in situ cell barcoding, the following are non-limiting examples of products that may be created:
A collection of cells containing precursor libraries and barcoding oligonucleotides, which have the ability to hybridize to each other due to complementary sequences on their 5′ ends, but that cannot amplify each other because the hybridization product creates 3′ overhangs.
A collection of cells in which adapters containing one or more universal sequences (e.g., read1 sequence, read2 sequence, P5 sequence, and/or P7 sequence) and a barcode sequence (degenerate/partially degenerate, or set of defined sequences) are added to (e.g., both sides) of genomic fragments/amplicons/RNA/cDNA.
An NGS library including fragments with sequencing adapters (e.g., P5 and/or P7 sequences) in which the progeny of each unique molecule may or may not have the same pair of cellular barcodes.

A barcode sequence can be added to a target nucleic acid of interest during amplification or ligation by carrying out PCR or ligation with a with the barcode sequence such that the barcode sequence is incorporated into the final amplified or ligated target nucleic acid product to produce cellular barcoded libraries. In some embodiments, the barcoding sequence is 4-20 base pairs in length, or 5-19 base pairs in length, or 6-18 base pairs in length, or 6-17 base pairs in length, or 6-16 base pairs in length, or 6-15 base pairs in length, or 6-14 base pairs in length, or 6-13 base pairs in length, or 6-12 base pairs in length, or 6-11 base pairs in length, or 6-10 base pairs in length or 6-9 base pairs in length, or 6-8 base pairs in length, or 6-7 base pairs in length. In a preferred embodiment, the barcoding sequence is 6-8 base pairs in length.

In some embodiments, the barcoding sequence can comprise a degenerate sequence. In some embodiments, the barcoding sequence is degenerate. In some embodiments the degenerate barcoding sequence is 6-8 base pairs in length. The entire barcoding sequence may be degenerate, where all nucleotides are randomized (e.g. a mixture of oligonucleotides of sequence N6, N7, or N8). The barcoding sequence may be partially degenerate where one or more nucleotides are randomized. The barcoding sequence may be degenerate at defined nucleotides. The barcoding sequence may contain nucleotide positions which only contain purines. The barcoding sequence may contain nucleotide positions which only contain pyrimidines. These barcoding sequences can act as unique molecular identifiers.

In other embodiments, rather than use a mixture of degenerate barcoding sequences, where the theoretical maximum number of barcodes of length ‘n’ is 4″, it is useful to include a smaller set of barcodes. The individual barcodes within such a set can be designed so that a single error does not convert one barcode into another member of the set. These designs, including an appreciation of minimal distance between different barcodes, can be used to permit error correction. Thus in some embodiments, the set of barcodes (e.g. being 6-20 nucleotides long, such as 6-10 nucleotides) have a Hamming distance of at least 2 (e.g. 3, 4, 5 or more); in other embodiments, the set of barcodes have a Levenshtein distance of at least 2 (e.g. 3, 4, 5 or more). Ideally, each barcode in the set differs form each other barcode by at least two nucleotides at corresponding sequence positions, to reduce the potential that cross-mutation of one barcode into another member of the set, and so that a single point mutation does not convert any single barcode into any other member of the set. Tools are available to facilitate the design of such barcode sets e.g. BARCOSEL (Somervuo et al. BMC Bioinformatics. 2018; 19:257), or the scripts described by Bystrykh (PLOS ONE 7 (5): e36852).

In some embodiments, after performing the lysing step, the method includes ligating the nucleic acid fragments with barcode adapter sequences. In certain embodiments, the barcode adapter sequences comprise a set of forward and/or reverse barcoding adapter sequences. In some embodiments, ligating the forward and/or reverse barcode adapter sequences occurs before the purifying step, or after the purifying in step.

For example, methods of cellular barcoding precursor libraries in situ include (a) contacting nucleic acid fragments within a cell suspension with: (i) a first set of barcoding oligonucleotides, each barcoding oligonucleotide comprising: a first barcode; two consensus regions, wherein the two consensus regions of each barcoding primer comprises: one of the two consensus regions comprises a nucleotide sequence that is complementary to a 5′ read region of a first strand of one of the DNA or RNA fragments, and the second of the two consensus regions comprises a first adapter sequence; (ii) a second set of barcoding oligonucleotides, each barcoding oligonucleotides comprising: a second barcode; two consensus regions, wherein the two consensus regions of each barcoding primer comprises: one of the two consensus regions comprises a nucleotide sequence that is complementary to a 5′ read region of a second strand of one of the DNA or RNA fragments, and the second of the two consensus regions comprises a second adapter sequence; (b) amplifying: the first set of barcoding oligonucleotides to produce a first set of barcoding primers; and the second set of barcoding oligonucleotides to produce a second set of barcoding primers; (c) amplifying the nucleic acid fragments with first and second set of barcoding primers to produce a set of amplicon products, wherein the set of amplicon products comprise the first barcoding primer bridging from the 5′ end of the 5′ strand of the nucleic acid fragments and the second barcoding primer bridging from the 5′ end of the opposite strand (3′ strand) of the nucleic acid fragments.

In some embodiments, wherein the first set of barcoding oligonucleotides, the second set of barcoding oligonucleotides, or both, comprise one or more modifications.

In some embodiments, the one or more modifications comprise one or more alpha-thiol dNTPs. In some embodiments, the one or more alpha-thiol dNTPs are selected from alpha-thiol-dTTP, alpha-thiol-dCTP, alpha-thiol-dGTP, and alpha-thiol-dATP. In some embodiments, the amplifying step (b) comprises performing the amplifying step using an alpha-thiol dNTP mix, thereby producing a first set of barcoding primers, a second set of barcoding primers, or a combination thereof, comprising one or more alpha-thiol dNTPs. In some embodiments, the alpha-thiol dNTP mix comprises an alpha-thiol-dTTP, an alpha-thiol-dCTP, an alpha-thiol-dGTP, or an alpha-thiol-dATP, or a combination thereof.

In some embodiments, the first set of barcoding oligonucleotides, second set of barcoding oligonucleotides, or both contain additional sequence for a primer binding site.

In some embodiments, the primer binding site is an amplification sequence. In some embodiments, step (i) further comprises contacting the first barcoding oligonucleotide with a first primer set comprising nucleotide sequences that is complementary to the amplification sequence.

In some embodiments, step (ii) further comprises contacting the second barcoding oligonucleotides with a second primer set comprising a nucleotide sequence that is complementary to the amplification sequence.

In some embodiments, the first set of barcoding oligonucleotides and the first primer set are annealed prior to said contacting to produce a first set of annealed barcoding oligonucleotides.

In some embodiments, the said amplifying in step (b) comprises amplifying via polymerase chain reaction, the first and second set of barcoding oligonucleotides with the first and second set of primers to produce the first and second barcoding primers.

In some embodiments, the said amplifying in step (b) comprises amplifying via isothermal amplification, the first and second set of barcoding oligonucleotides with the first and second set of primers to produce the first and second barcoding primers.

In some embodiments, the first set of barcoding oligonucleotides and the first primer set are not annealed prior to said contacting.

In some embodiments, step (i) further comprises contacting the first barcoding oligonucleotide with a first primer set comprising nucleotide sequences that are complementary to the adapter sequence of the first barcoding oligonucleotides.

In some embodiments, step (ii) further comprises contacting the second barcoding oligonucleotides with a second primer set comprising a nucleotide sequence that is complementary to the second adapter sequence of the second set of barcoding oligonucleotides.

In some embodiments, the nucleic acid fragments are not amplified during step (b). In some embodiments, the first and second barcoding oligonucleotides comprise hairpin barcoding oligonucleotides. In some embodiments, the DNA is a double-stranded DNA (dsDNA) fragment.

### Isothermal Amplification and PCR Amplification Reactions

In some embodiments, isothermal amplification is performed to produce the set of amplified barcode oligonucleotide primers before using PCR to amplify the prepared DNA, cDNA, or RNA fragments within the cell populations to produce cell barcoded libraries. In some embodiments, a nicking enzyme, an isothermal polymerase, first set of annealed cellular barcoding oligonucleotides (e.g. annealed to the first set of amplification primers), and the second set of annealed barcoding oligonucleotides (e.g., annealed to the second set of amplification primers) are added to cells with prepared nucleic acid fragments.

In some embodiments, the first and second set of barcoding oligonucleotides and the first and second set of amplification primer are added separately.

In alternative embodiments, the first and second set of barcoding oligonucleotides comprise hairpin oligonucleotides that contains both the barcoding oligonucleotides and amplification primers in addition to a hairpin sequence (e.g., a stem loop sequence) in a single molecule. In some embodiments, a first set of hairpin barcoding oligonucleotides comprise a first barcode (e.g., molecular cellular label); and a consensus region comprising a nucleotide sequence that is complementary to a 5′ read region of a first strand of the DNA, cDNA or RNA fragments. In some embodiments, the second set of hairpin barcoding oligonucleotides comprises a second barcode (e.g., molecular cellular label); and a consensus region comprising a nucleotide sequence that is complementary to a 5′ read region of a second strand of the DNA, cDNA, or RNA fragments.

In some embodiments, the hairpin barcoding oligonucleotides in the first set of hairpin barcoding oligonucleotides optionally includes a first adapter sequence (e.g., a P5 or P7 sequence), and the hairpin barcoding oligonucleotides in the second set of hairpin barcoding oligonucleotides optionally includes a second adapter sequence (e.g., a P5 or P7 sequence). The first and second set of hairpin barcoding oligonucleotides optionally include cleavage sites. In some embodiments, the hairpin oligonucleotides comprise a hairpin sequence at the 5′ or 3′ end of the barcoding oligonucleotide (e.g. stem loop). Such embodiments with hairpin oligonucleotides may be alternatives to annealed cellular barcoding oligonucleotides/amplification primers.

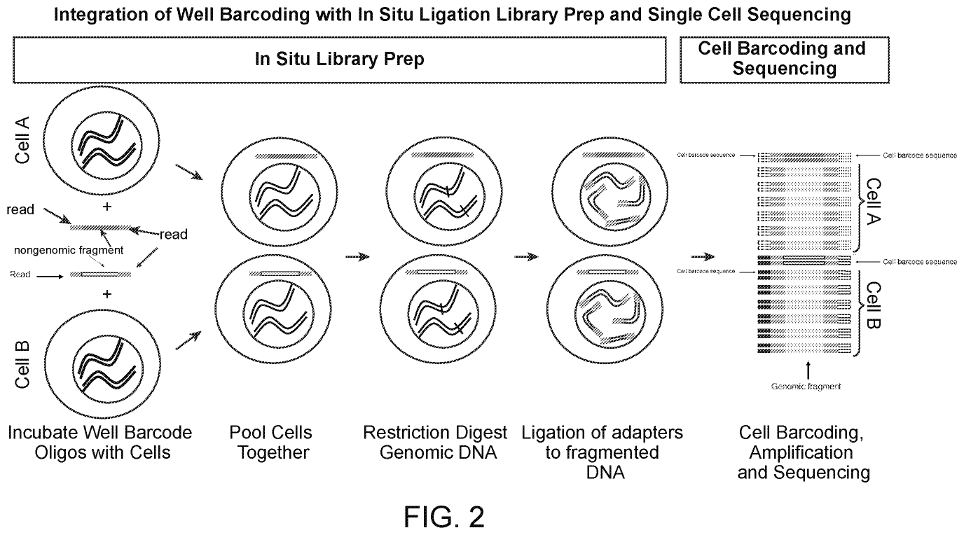

For example, during an isothermal amplification reaction, the isothermal polymerase amplifies the barcoding oligonucleotides and the nicking enzyme recognizes the ERS cleaving only one of the strands of the dsDNA and allowing priming for subsequent amplification of the barcode oligonucleotide and release of amplified barcoding oligonucleotide. The resulting barcoding products (barcoding primers) is the reverse complement of the barcoding oligonucleotide without the ERS site, and comprises: 5′-CR3-DS′-CR1-3′ (“E” of FIG. 2A″ and 5′-CR4-DS′-CR2-3′ (“F” of FIG. 2A) of PCT Application Publication No.: WO2022/192603.

After the isothermal amplification reaction is performed in the partitions, and the isothermal amplification enzyme and nicking enzymes are heat inactivated, if required, a PCR amplification reaction is performed on the cells. The PCR template (precursor libraries) PCR barcoding primers (isothermally amplified barcode oligonucleotides) and PCR amplification reagents are already present in the partitions. During PCR amplification, the dsDNA fragments are denatured or displaced. Following denaturing or displacement, the isothermally amplified barcode primers are annealed and extended in 5′-3′ direction along the DNA fragments. In some embodiments, this process is repeated, via one or more, two or more, three or more, three or more, four or more, five or more, six or more, seven or more, eight or more, nine or more, or ten or more PCR cycles, to ensure that the amplicons contain cell barcode sequences on both sides of the insert. The annealing and extending steps result in a set of amplicon products, containing a duplex molecule where the first strand contains 5′-CR3-DS′-CR1-Insert-CR2′-DS-CR4′-3′ (FIG. 2A) and second strand contains 5′-CR4-DS-CR2-Insert′-CR1′-DS-CR3′-3′ (FIG. 2A).

After the PCR amplification step, the DNA fragments contain all of the required information to associate the sequence read back to the cell it originated from and therefore partitions can be pooled and cells can be lysed, if necessary. If CR3 and CR4 adapter sequences contained all of the required sequences for amplifying on the flow cell the material can be sequenced or further processed in any ways that adapter sequence-labeled DNA fragment would be used (i.e., can undergo hybrid capture target enrichment protocols, and the like.)

If CR3 and CR4 are not sufficient for amplifying on the flow cell, another PCR amplification reaction may be performed, for example, in vitro. This step can add indexing primers to the amplicons and then the material can be sequenced or further processed in any ways that adapter labeled DNA fragment would be used (i.e., can undergo hybrid capture target enrichment protocols, and the like).

A non-limiting example of the workflow of the isothermal amplification and PCR workflow for cellular barcoding in situ is shown in FIGS. 2A and 2B of PCT Application Publication No.: WO2022/192603, which is hereby incorporated by reference in its entirety. Inputs of the Isothermal amplification reaction include: A. In Situ Insert Library with Consensus regions (CR1 and CR2) appended to DNA; B. Annealed isothermal amplification primer set 1, that includes a barcode oligonucleotide 5′-CR1′-DS (degenerate sequence)-CR3′-3′ and barcode amplification primer 5′-ERS-CR3-3′; C. Annealed isothermal amplification primer set 2, that includes barcode oligonucleotide 5′-CR2′-DS-CR4′-3′ and barcode amplification primer 5′-ERS-CR4-3′; and the nicking enzyme and isothermal DNA polymerase. The products that come out of the isothermal amplification reaction include: D. In Situ Insert Library with Consensus regions appended to DNA, exactly same as A; E. Amplified Barcode Oligo Set 1, generated via isothermal amplification of the annealed isothermal amplification primer set 1 (B), where the Barcode oligo extends through the ERS and the barcode amplification primer extends through the DS and CRI regions. The nicking enzyme can cleave (repeatedly) the top strand of the ERS and allow the isothermal amplification enzyme to extend the ERS over the barcode oligo; F. Amplified Barcode Oligo Set 2, generated via isothermal amplification of the annealed isothermal amplification primer set 2 (C), where the Barcode oligo extends through the ERS and the barcode amplification primer extends through the DS and CR2 regions. The nicking enzyme can cleave (repeatedly) the top strand of the ERS and allow the isothermal amplification enzyme to extend the ERS over the barcode oligo. FIG. 2A of PCT Application Publication No.: WO2022/192603describes the next step requiring PCR Amplification on the cells that have undergone isothermal amplification of the barcoding oligonucleotides. The inputs include cells containing the products from FIG. 2A of PCT Application Publication No.: WO2022/192603, and the outputs include complete libraries with two sets of degenerate sequences, both surrounded by consensus regions.

In some embodiments, isothermal amplification is performed to produce amplified primers (e.g., a first primer and a second primer) where the primers do not include barcode sequences. In some embodiments, a nicking enzyme; an isothermal polymerase; an oligonucleotide comprising an amplification sequence and a consensus region; and an amplification primer comprising a nick endonuclease recognition site or reverse complement thereof and a nucleotide sequence that is at least partially complementary to the amplification sequence on the oligonucleotide are added to a reaction container (e.g., any of the reaction containers provided herein or known in the art). An isothermal amplification reaction generates the primer comprising the reverse complement of the consensus region.

In some embodiments, a nicking enzyme; an isothermal polymerase; an oligonucleotide comprising an amplification sequence and a consensus region; an amplification primer comprising a nick endonuclease recognition site or reverse complement thereof and a nucleotide sequence that is at least partially complementary to the amplification sequence on the oligonucleotide; a second oligonucleotide comprising a second nick endonuclease recognition site or reverse complement thereof; and a second amplification primer comprising a second nick endonuclease recognition site or reverse complement thereof and a nucleotide sequence that is at least partially complementary to the second amplification sequence on the second oligonucleotide are added to a reaction container (e.g., any of the reaction containers provided herein or known in the art). An isothermal amplification reaction generates the primer comprising the reverse complement of the consensus region and the second primer comprising the reverse complement of the second consensus region.

In some embodiments, the first and second barcodes each comprises a degenerate nucleotide sequence. In some embodiments, the first and second barcodes each comprises a partially degenerative nucleotide sequence. In some embodiments, the degenerate sequence comprises 8-50 nucleotides. In some embodiments, the degenerate sequence comprises 8-20 nucleotides. In some embodiments, the set of first and set of second barcoding oligonucleotides consist of pooled barcoding oligos with multiple different defined sequences.

In some embodiments where the method of amplifying the barcoding oligonucleotide or non-barcoding oligonucleotides include isothermal amplification, the isothermal amplification is performed using an isothermal polymerase. Non-limiting examples of isothermal polymerases include Klenow Fragment (Exo-), Bsu Large Fragment, Bst DNA polymerase, Bst2.0, Sequenase, Bsm DNA Polymerase, EquiPhi29, and Phi29 DNA polymerase, Bst, Bst3.0, IsopolSD+.

In some embodiments where the method of amplifying the barcoding oligonucleotide or non-barcoding oligonucleotides include isothermal amplification, the amplification is performed under conditions that allow for primer invasion.

In some embodiments, the set of first and set of second barcoding oligonucleotides consist of pooled barcoding oligos with multiple different defined sequences.

In some embodiments, the first and second barcodes each comprises 8-50 nucleotides.

In some embodiments, the two consensus regions of the first barcoding oligonucleotides flank the first barcode.

In some embodiments, the two consensus regions of the second barcoding oligonucleotides flank the second barcode.

In some embodiments, the nucleotide sequence of the first or second barcode is positioned between the nucleotide sequences of the two consensus regions.

In some embodiments, the degenerate sequence of each first and second barcode is distinguishable from one another.

In some embodiments, the first barcode of the barcoding oligonucleotides within the first set of barcoding oligonucleotides is distinguishable from other first barcodes of the first set of barcoding oligonucleotides by its nucleotide sequence.

In some embodiments, the second barcode of the barcoding oligonucleotides within the second set of barcoding oligonucleotides is distinguishable from other second barcode of the second set of barcoding oligonucleotides by its nucleotide sequence

In some embodiments, (i) the adapter sequence of the first set of oligonucleotides comprises a P5 adapter sequences or a reverse complement thereof, and the adapter sequence of the second set of oligonucleotides comprises a P7 adapter sequences or a reverse complement thereof, or (ii) the adapter sequence of the first set of oligonucleotides comprises a P7 adapter sequences or a reverse complement thereof, and the adapter sequence of the second set of oligonucleotides comprises a P5 adapter sequences or a reverse complement thereof.

In some embodiments, the method further comprises, after step (c) contacting the amplicon product with a set of indexing primers, and performing an amplification reaction to produce a second set of amplicon products.

In some embodiments, performing isothermal amplification and PCR amplification occurs in the same buffer. In some embodiments, amplifying via isothermal amplification and amplifying via PCR amplification in the partition occur in a single reaction container comprising the buffer.

In some embodiments, the buffer is not aspirated, washed, or modified between isothermal amplification and PCR amplification steps. In some embodiments, the buffer is aspirated, washed, or modified between isothermal amplification and PCR amplification steps.

In some embodiments, the buffer is selected from one or more of: Isothermal Amplification Buffer (NEB, 20 mM Tris-HCl, 10 mM (NH4)2SO4, 50 mM MgSO4, 0.1% Tween20, pH8.8 at 25° C.); NEB Buffer 2 (NEB, 50 mM NaCl, 10 mM Tris-HCl, 10 mM MgCl2, 1 mM DTT, pH7.9 at 25° C.); Phi29 Reaction Buffer (NEB, 50 mM Tris-HCl, 10 mM MgCl2, 10 mM (NH4)2SO4, 4 mM DTT, pH7.5 at 25° C.); Taq Polymerase Reaction Buffer (10 mM Tris-HCl, 50 mM KCl, 1.5 mM MgCl₂, pH 8.3 at 25° C.); Unnamed Buffer 1 (25 mM TAPS-HCl, 50 mM KCl, 2 mM MgCl2, 1 mM β-mercaptoethanol, pH 9.3 at 25° C.); Isothermal Amplification Buffer II (NEB, 20 mM Tris-HCl, 10 mM (NH4)2SO4, 150 mM KCl, 2 mM MgSO4, 0.1Tween® 20, pH 8.8 at 25° C.; Thermopol Reaction Buffer (NEB, 20 mM Tris-HCl, 10 mM (NH4) 2SO4, 10 mM KCl, 2 mM MgSO4, 0.1% Triton® X-100, pH 8.8 at 25° C.); TaqDNA polymerase PCR Buffer (20 mM Tris HCl, 50 mM KCl, pH 8.4 at 25° C.); Equinox Buffer (Watchmaker Genomics); phi29 Pol Reaction buffer (Watchmaker Genomics); Sequenase Reaction Buffer (Applied Biosystems); IsopolSD+ Reaction Buffer (ArcticZymes); Q5 Reaction Buffer (NEB); and Buffer A (Stilla Genomics), KAPA HiFi DNA Polymerase Buffer (Roche).

In some embodiments, isothermal amplification and PCR amplification occurs in a single reaction container. In some embodiments, a buffer used during isothermal amplification and PCR amplification is not washed, removed, or modified during amplification steps.

Sample Pooling before In Situ Library Preparation of Precursor Libraries

Another aspect of the present methods includes sample pooling. Pooling samples together as early as possible provides a method for reducing reagent use, handling costs, sample variability, and allows for higher throughput and single cell resolution. Pooling samples on, e.g., wells or tubes, also helps get single cell resolution to identify where the cells came from after sequencing. This disclosure features methods where, prior to in situ library preparation steps, cell samples can be pooled to identify where cells came from (e.g., from a particular well or tube).

As used herein, a “well” is defined as specific sample provided from a culture tube, microfuge tube, PCR tube, a specific well in a culture plate, well in a PCR plate, or a coordinate on a slide.

As used herein, a “Unique well identifier (UWI)” is a DNA molecule (e.g., single stranded or double stranded DNA molecule) that can be amplified at the appropriate stage in order to append a cell identifier.

As used herein, a “Cell Identifier” is a unique cell barcode or combination of cell barcodes that can be used to identify which reads belong together.

As used herein, “Spike-In fragments” are nucleic acid fragments that can be single stranded or double stranded, circular or linear. These fragments contain DNA non-native to the cells with barcode sequences that can identify them from similar sequences and are added to the cells such that some of them get into the cells. In some embodiments, a spike-in-fragment can be a UWI. A variety of conventional methods can be used to synthesize these fragments, and include oligonucleotide synthesis, molecular cloning methods, and PCR amplification.

As used herein, a “genomic fragment” is the product of library preparation. For example, in this application, it refers to the product of multiplex PCR amplification, enzymatic fragmentation, tagmentation, or RNA reverse transcription.

Sample pooling in the present disclosure includes adding a unique well identifier (UWI) into a well or tube containing the cellular sample that gets incubated with the cells in individual wells and enters the cells, and then mixing the wells together. UWIs are not directly conjugated to the nucleic acid fragments (e.g., genomic region of interest/genomic fragments) from the cells, but instead, with the aid of single cell sequencing, are associated with the same cell identifier as the nucleic acid fragments. The UWI is not disrupted or acted by the steps of enzymatic fragmentation of the in situ library preparation process, but does go through the normal process of the library preparation steps such as amplification throughout library preparation. The method results in obtaining sequencing reads labeled with cell barcodes from the cells with cell barcodes attached to the cells, as well as sequencing reads labeled with cell barcodes from the UWI within the cells. In this case, the UWI is in place of the genomic insert sequence of the genomic sequence. Both are associated with a cell barcode, the same barcode if they are from the same cell, thereby creating a map to identify which cells belong to which well or tube and track where the cell within the sample came from. In some embodiments, multiple cells originating from the same well will have the same UWI, but different cell identifiers. UWI is not physically linked or attached to the target genomic fragments, and are separate, distinct molecules.

In some embodiments, UWI are added to the cell samples in each well before any library preparation steps. Performance of ligation-based or amplicon-based library preparation steps shown as equivalent steps in PCT/US2021/046025, which is hereby incorporated by reference in its entirety. In certain embodiments, the UWI are added to the cell sample after cells are fixed. In certain embodiments, the UWI are added to the cell sample after cells are fixed and permeabilized. In some embodiments, UWIare added to the cell sample before fixation and permeabilization.

Using the typical NGS sample barcodes as UWIs, would be possible, but would require a number of manipulations before introduction. These barcodes are physically linked together and would not benefit from the efficiencies of sample pooling. In some embodiments, in the case of an in situ DNA ligation library preparation protocol, UWI incorporation (as a sample barcode) can occur during the ligation step, after cell fixation, permeabilization, and enzymatic fragmentation. In some embodiments, in the case of in situ amplicon library preparation, UWI incorporation can occur after the targeting PCR, after cell fixation and permeabilization. In situ cellular barcoding steps can be found in PCT/US2023/062776, which is hereby incorporated by reference in its entirety.

In some embodiments, a spike in fragment is a UWI, identifying from where the cell sample has originated. It can act as a sample barcode which is added before in situ library preparation steps, and can be combined with cell barcodes and additional sample barcodes.

In some embodiments, the UWI introduced to the cellular sample is resistant to the enzymatic steps that may render the UWI incapable of performing its function as a well identifier. In such embodiments that include UWI resistance to enzymatic fragmentation, the UWI remains amenable to other required enzymatic steps, such as ligation and/or amplification.

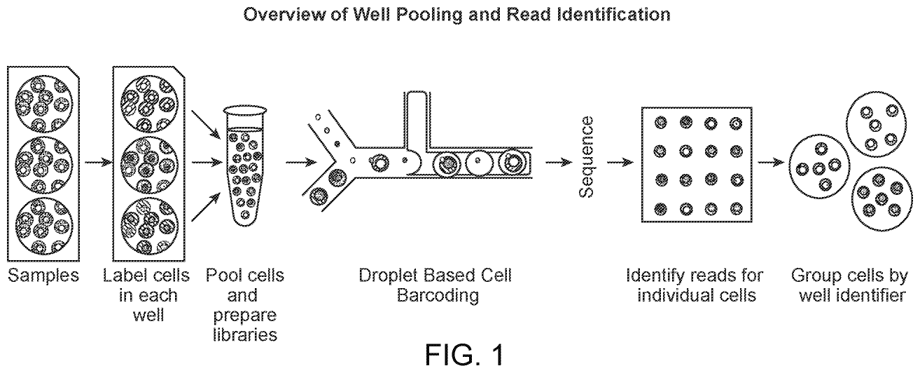

FIG. 1 shows 3 sets of samples in 3 different wells. Each well containing cellular samples is incubated with a distinct UWI (striped and open transparent inner circle, completed shaded inner closed circle, or striped and completely shaded circle) as shown by the 3 different colors. All samples are then pooled into a tube and undergo library preparation steps, and/or droplet based cell barcoding. Following sequencing, reads for individual cells can be identified and traced back by the well identifier.

FIG. 2 shows integration of well barcoding with in situ ligation based library preparation and single cell sequencing. As shown, cell A of a first well is incubated with a first UWI that contains a non-genomic fragment (dark grey), and two barcode reads (grey), and cell B of a second well is incubated with a second UWI that contains a non-genomic fragment (light grey with black outline), and two barcode reads (grey). The samples (cell A and cell B) are then pooled together and undergo in situ library preparation steps, such as restriction digest of the genomic DNA and ligation of adapters to fragmented DNA (shown in light grey, without black outline). The adapters can be the same adapters as shown in the UWI (grey). As shown, the restriction digest step does not fragment either of the first or second UWIs. Additionally, the UWI are not adhered or attached to the genomic fragmented DNA (fragmented DNA is shown in light grey without outline). Following in situ library preparation, cell barcoding (e.g., incorporating cell barcodes such as a unique cell identifier), amplification, and sequencing is performed on the genomic DNA (light grey without outline) and on the UWI (dark grey strand in cell A and light grey with black outline strand in cell B). After sequencing, cells A can be associated with the first UWI and contain the same cell barcode sequences (black short strand and white with black outline short strand) as the genomic fragment (light grey strands without black outline), thereby can be mapped to the cells of the first well. Cell B can be associated with the second UWI and contain the same cell barcode sequences as the genomic fragment (light grey strands without black outline), thereby can be mapped to the cells of the second well.

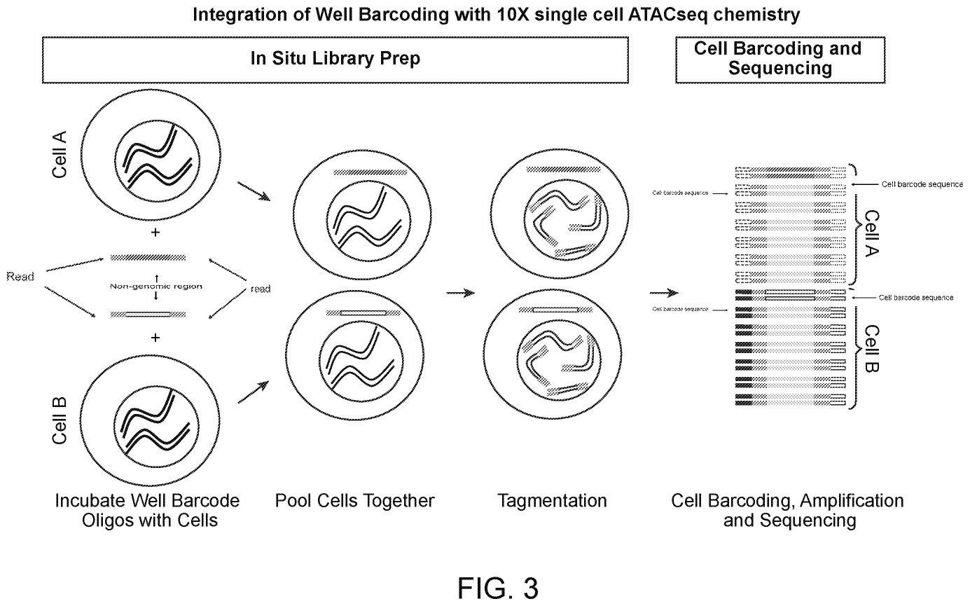

FIG. 3 shows integration of well barcoding with 10X single cell ATACseq chemistry. As shown, cell A of a first well is incubated with a first UWI that contains a non-genomic fragment (dark grey), and two barcode reads (grey short strands), and cell B of a second well is incubated with a second UWI that contains a non-genomic fragment (light grey with black outline), and two barcode reads (grey short strands). The samples (cell A and cell B) are then pooled together and undergo in situ library preparation steps, such as tagmentation. Following in situ library preparation, cell barcoding (e.g., incorporating cell barcodes such as a unique cell identifier), amplification, and sequencing is performed on the genomic DNA (light grey strands without black outline) and on the UWI (dark grey strand in cell A and light grey strand without black outline in cell B). After sequencing, cells A can be associated with the first UWI and contain the same cell barcode sequences (dashed strands) as the genomic fragment (light grey strands without black outline), thereby can be mapped to the cells of the first well. Cell B can be associated with the second UWI and contain the same cell barcode sequences (black short strand and white short strand with black outline) as the genomic fragment (grey strands without outline), thereby can be mapped to the cells of the second well.

In some embodiments, the cell identifier is added during cell barcoding, where every cell has its own barcode. The unique cell identifier such as a cell barcode is physically attached to the reads coming from the cell. The unique cell identifier such as a cell barcode are also physically attached to the reads coming from the UWI.

In some embodiments, a unique well identifier is added to the sample mixture containing precursor libraries, and undergoes the same steps as the precursor library. For example, the method can include preparing a mixture in one or more containers comprising one or more of: the cell population, cell barcoding oligonucleotides, amplification reagents, and one or more unique well identifiers; partitioning the mixture into a plurality of partitions, wherein at least some partitions contain: a cell from the cell population comprising the precursor library, a plurality of cell barcoding oligonucleotides, and one or more unique well identifiers; barcoding the precursor libraries in the plurality of partitions by: amplifying the cell barcoding oligonucleotides in the at least some partitions to produce cell barcoding primers; then amplifying the unique well identifiers and precursor libraries in partitions with the cell barcoding primers to produce cell barcoded libraries and cell barcoded unique well identifiers; and isolating the cell barcoded libraries and cell barcoded unique well identifiers.

In some embodiments, the UWIs are added to the mixture of cells before enzymatic fragmentation of library preparation of precursor library. Thus, UWIs are resistant, or at least partially resistant to the enzymatic steps performed during enzymatic fragmentation but permit other enzymatic steps to occur such as ligation and/or PCR amplification.

Sample pooling may be useful for sample tracking, and samples can be different cell types, same cell type, but different sample, different heterogenous samples, and the like. Sample pooling allows pooling of samples well before any enzymatic steps, dramatically decreasing library prep costs. For example, if a sample is from a different well, then it should get a different well barcode.

Structure of Spike-In ssDNA or dsDNA Fragment

In some embodiments, the spike-in DNA fragment comprise ssDNA or dsDNA. Spike-in Fragments can be linear or circular. In some embodiments, two universal sequences flank the barcoding insert fragment (non-genomic DNA). These universal sequences aid amplification of the spike-in DNA fragment, and in some embodiments, comprise Illumina R1 and R2 sequences, or their reverse complements. Specific sequences is determined by single cell isolation method and/or library prep method.

In some embodiments, the barcoding insert is designed to be similar in length to the target insert size of the library prep method. Depending on library preparation method, and sequencing platform the barcoding insert can range from 10-10,000 bp. Illumina sequencing will target 50-350 bp, while long-read technologies will require larger insert sizes. In some embodiments, the entire barcoding insert sequence can encode the unique well identifier (UWI) allowing for a designated edit distance. In certain embodiments, the barcoding insert includes one or more UWIs near either or both of the insert ends. These UWIs can be smaller, 4-20 bp, and positioned near (within 0-50 bp) of the ends of the barcoding insert fragment. In certain embodiments, the UWI in the barcoding insert are not contiguous. In certain embodiments, the UWI in the barcoding insert are contiguous.

Positioning the barcode sequence near the beginning of the read will allow sequencing of different lengths to be performed. The remaining sequence comprise of a sequence absent from the genomic sequence(s) of the organism(s) being sequenced.

Nuclease Resistant Spike-In ssDNA Fragment

In situ ligation library prep methods described in PCT/US2021/046025 use enzymatic fragmentation to prepare insert fragments of appropriate sequencing lengths. Generation of these fragments can be done with the various enzymes mentioned in that PCT/US2021/046025, some of which only recognize dsDNA. But others (i.e. DNaseI and micrococcal nuclease) have activity on both ssDNA and dsDNA. In this case, modifications to the spike-in ssDNA fragment to confer nuclease resistance could be made to the spike-in oligonucleotide fragment. One modification, phosphorothioate bonds substitutes a sulfur atom of a non-bridging oxygen in the phosphate backbone. Providing this phosophorthioate modification at every position, or every other position, or further apart with 0-10 normal bounds between each phosphorothioate bonds, will reduce the digestion of this molecule during enzymatic fragmentation. Phosphorothioate bonds before all A's, C's, G's, or T's, or a combination of bases, or a random distribution of phosphorthioate bonds throughout the A's, C's, G's, or T's, or a combination of bases could also work. Alternative methods are to include locked nucleic acids (LNAs) or a combination of methods. Further composition information follow Embodiment 1.

Structure of Spike-In dsDNA Fragments

In some embodiments, spike-in ssDNA fragments are designed to resist nuclease digestion. However, an alternative approach is to embrace nuclease digestion and provide a molecule large enough to handle the effect of digestion. For the in situ ligation protocol described in PCT/US2021/046025, a dsDNA fragment can be used as the spike-in fragment. The spike-in dsDNA fragment can be linear (ie a geneblock, digested plasmid), or circular (i.e. plasmid) and can be from 200-10,000 bp, with one or more 6-20 bp UWI present throughout the spike-in dsDNA strand. Enzymatic fragmentation will cleave the spike-in dsDNA fragment and then the ligation step will ligate on universal adapters, making the product amplifiable. As with Embodiment 1, the backbone sequence is absent from the genomic sequence(s) of the organism(s) being sequenced.

### Structure of Spike-In RNA Fragment

In some cases, a spike-in fragment comprises an RNA fragment. In certain embodiments, RNA may be more desirable than DNA. For example, in such embodiments where RNA is used as the spike-in fragment molecule, the cell barcoding method directly labels the RNA with the cell barcode, as occurs with some single RNA kits (e.g. 10× genomics). In this method, RNA fragments are structured and modified such that the reverse transcriptase primers can amplify the RNA fragment. Either methods to enrich or retain the spike-in fragment during library preparation will occur, or some of the non-enriched sample need to be sequenced to associate well barcodes with cell barcodes.

### Spike-In Fragment Timing—Before Fixation

In some embodiments, spike-in fragments is added before any protocol perturbations. However, the intact cell membrane and/or cell wall of cells is designed to prevent free diffusion across the membrane. Delivery systems for oligo-nucleotide probes have been developed for detection of RNA in live cells (doi: 10.1146/annurev-bioeng-061008-124920). Similar methods can be used to introduce our spike-in DNA fragments and include conventional methods such as that described in doi: 10.1146/annurev-bioeng-061008-124920:
1) Cell membrane permeabilization with streptolysin O, a reversable permeabilizer, or other bacterial exotoxins. Incubation of the cells with streptolysin O and the spike-in fragment in a buffer lacking Ca2+, will allow for influx of the spike-in fragment before cell fixation and permeabilization. Cells can then be resealed by added Ca2+ to the incubation mix (https://doi.org/10.1073/pnas.051429498). This will allow pooling cells together with minimal cross-contamination between other samples.
2) Conjugation of cell penetrating peptides to the spike-in DNA fragment. Cell penetrating peptides have been shown to allow uptake of the peptides and their payload in mammalian systems (https://doi.org/10.1038/s42003-021-01726-w), yeast (https://doi.org/10.1016/j.febslet.2005.07.099), and is likely conserved across eukaryotic organisms. These peptide sequences can be conjugated to DNA oligos allowing for the DNA to permeate the cell membranes. This is an effective method for spike-in DNA fragment introduction.
3) Use of electroporation to introduce spike-in DNA fragments. Electroporation is a traditional method for introducing DNA fragments into cells (doi: 10.1146/annurev-bioeng-061008-124920) and can be harnessed to introduce these spike-in DNA fragments.
   Spike-In DNA Fragment after Fixation and/or Permeabilization

In some embodiments, pooling samples after the fixation and/or permeabilization steps bypass the need for developing methods to get DNA fragments into the cells. After fixation and permeabilization, the cells readily uptake enzymes and oligos presented in appropriate concentrations as described in PCT/US2021/046025, thus the spike-in DNA fragments can be provided during an incubation at the appropriate concentration (1 pM to 1 uM). After incubation, the cells are pelleted and washed to remove excess non-cell associated spike-in DNA fragments. After washing, the cells can be pooled together and the remaining steps of in situ library prep can be performed.

Transformation of Spike-In dsDNA Fragment

Spike-in DNA fragments can be transformed into the cells of a well, creating a cell line, in which the progeny contain the spike-in DNA fragment. This can be used with dsDNA, specifically circular DNA molecules that are constructed as plasmids, but could also include dsDNA fragments if appropriately designed. Notably these lines become modified from the original sample through the addition of this exogenous sequence, but the UWI is present within it. Chemical and electroporation methods to make the cells susceptible for fragment uptake can be used.

### Isolating the Cell Barcoded Libraries

An aspect of the present methods include isolating the cell barcoded libraries. In some embodiments, the method comprises lysing the cells containing the set of amplicon products. In some embodiments, the method comprises breaking emulsions and purifying the DNA or RNA.

Methods of isolating cell barcoded library are conventionally known in the art. For example, cell barcoded libraries can be isolated by breaking the partition, lysing the cell in the partition, and the like. Additional methods for breaking the partitions include freezing with liquid nitrogen, chloroform extraction, phenol chloroform extraction, and the like.

### Lysing the Cells

In some embodiments, the method comprises lysing the cells to collect cell barcoded libraries or nucleic acid fragments after partitioning. In certain embodiments, lysing the cells includes contacting the cells with a cell lysing agent. The lysing step can be accomplished by contacting the nucleic acid fragments or cellular barcoded libraries within the cell with a cell lysing agent or physically disrupting the cell structure. In some embodiments, cell lysis occurs during cellular barcoding. In some embodiments, cell lysis occurs before cellular barcoding. In some embodiments, cell lysis after cellular barcoding (e.g. lysis on cell barcoded libraries).

In some embodiments, lysing occurs after one or more PCR steps. Lysing the cells with a cell lysing agent facilitates purification and isolation of the nucleic acid fragments for each cell population.

In some embodiments, the lysing step of the present methods occurs after cellular barcoding and thus on the final amplicon products such as the second or third set of amplicon products. In some embodiments, lysing the cells purifies the amplicon products for each cell population.

In some embodiments, the lysing step of the present methods occurs after producing the second set of amplicon products (e.g., nucleic acid fragments) or for hybridization capture methods, after amplification used for population cell barcoding. In some embodiments, lysing the cells purifies the second set of amplicon products for each cell population.

In some embodiments, lysing the cell includes contacting the cells with a cell lysing agent.

Non-limiting examples of cell lysing agents include, but are not limited to, an enzyme solution. In some embodiments, the enzyme solution includes a proteases or proteinase K, phenol and guanidine isothiocyanate, RNase inhibitors, SDS, sodium hydroxide, potassium acetate, and the like. However, any known cell lysis buffer may be used to lyse the cells within the one or more cell populations.

Non-limiting examples of cell lysing methods include, but are not limited to, an enzyme solution-based method, mechanical based methods, physical manipulation, or chemical methods. In some embodiments, the lysis solution includes a proteases or proteinase K, phenol and guanidine isothiocyanate, RNase inhibitors, SDS, sodium hydroxide, potassium acetate, and the like. However, any known cell lysis buffer may be used to lyse the cells within the one or more cell populations. Mechanical lysis methods include breaking down cell membranes using shear force. Examples of mechanical lysis methods include, but are not limited to, using a High Pressure Homogenizer (HPH) or a bead mill (also known as the bead beating method). Physical methods include thermal lysis, such as repeated freeze thaws, cavitation, or osmotic shock. Chemical denaturation includes use of detergents, chaotropic solutions, alkaline lysis, or hypotonic solutions. Detergents for cell lysis can be ionic (anionic or cationic) or non-ionic detergents, or mixtures thereof. Examples of non-ionic detergents used for lysis include, but are not limited to, 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), and Triton X-100. A non-limiting example of an ionic detergent used for lysis includes, sodium dodecyl sulfate (SDS). Examples of chaotropic agents include, but are not limited to, ethylenediaminetetraacetic acid (EDTA), and urea.

In some embodiments, lysing includes heating the cells for a period of time sufficient to lyse the cells. In certain embodiments, the cells can be heated to a temperature of about 25° C. or more, 30° C. or more, 35° C. or more, 37° C. or more, 40° C. or more, 45° C. or more, 50° C. or more, 55° C. or more, 60° C. or more, 65° C. or more, 70° C. or more, 80° C. or more, 85° C. or more, 90° C. or more, 96° C. or more, 97° C. or more, 98° C. or more, or 99° C. or more. In certain embodiments, the cells can be heated to a temperature of about 90° C., 95° C., 96° C., 97° C., 98° C., or 99° C.

### Purifying the Cell Barcoded Libraries

Techniques for purifying cell barcoded libraries amplicon products are well-known in the art and include, for example, using size selection based magnetic bead purification reagent (e.g., Solid Phase Reversible Immobilization (SPRI) beads), passing through a column, phenol chloroform and the like. In some embodiments, purifying ligated cell barcoded libraries can include using magnetic streptavidin beads, for example if the cell barcoded libraries contain biotin. In some embodiments, the bead purification method uses Solid Phase Reversible Immobilisation (SPRI) beads. In some embodiments, the purification beads are made from polysterene—magnetite. These beads can be coated with negatively charged carboxyl groups. Bead-based size purification can include a step which involves the addition of an appropriate amount of salt (Na+) to aid in the precipitation of the DNA/RNA of the cell barcoded libraries. This bead-based size purification method can also include a size selection step. The bead-based purification method can also include an elution step through the addition of an aqueous solution. Examples of aqueous solutions for elution include, but are not limited to, water, nuclease free water, and Tris-EDTA. In some embodiments, the beads are magnetic beads. These beads can bind to the DNA/RNA in a pH dependent manner. The magnetic beads may be positively charged at low pH, and negatively charged at high pH. The pH of the DNA/RNA sample may be controlled to allow DNA/RNA binding to beads or its release from the beads. In some embodiments, the column based purification is silica based. This may require the presence of chaotropic salts. An non-limiting example of a chaotropic salt is guanidine hydrochloride. The chaotropic salt may be present in high quantities. The column based purification may involve one or more wash steps with an appropriate buffer. Examples of appropriate buffers include, but are not limited to, salt and/or ethanol solutions. The DNA/RNA can then be eluted in an appropriate elution buffer. Examples of appropriate elution buffers include, but are not limited to, water, nuclease free water, and Tris-EDTA. The DNA/RNA would usually be eluted under low salt conditions. In some embodiments, the phenol-chloroform purification method involves adding the phenol-chlorform mixture to equal volume of the DNA/RNA sample. Phenol-chloroform purification involves the extraction of DNA/RNA through isolation of the aqueous phase. The phenol-chloroform purification procedure can be repeated one or more times to increase the purity of the DNA/RNA. In some embodiments, the phenol: chloroform ratio in the phenol-chloroform mixture is made close to a 1:1 ratio. In some embodiments, the phenol-chloroform mixture also contains alcohol. An example of an alcohol which can be used is isomyl alcohol. An appropriate amount of isomyl alcohol is added to the phenol-chloroform mixture. The phenol: chloroform: isomyl alcohol ratio can be approximately 25:24:1. In some embodiments, the phenol-chloroform mixture is buffered. The phenol-chlorform purification method may include an additional ethanol precipitation step. The ethanol precipitation step involves isolating the DNA/RNA in a precipitate.

In some embodiments, purifying the ligated DNA or RNA fragments of the present methods creates an enriched or purified cell barcoded library for sequencing. The term “enriched” as used herein and in its conventional sense, refers to isolated nucleotide sequences containing the genomic regions of interest (e.g., target regions) using known purification techniques (e.g., hybridization capture, magnetic bead purification techniques, and the like). The purified libraries described in the methods herein includes the final purified library before sequencing.

In some embodiments, the purifying step includes bead purification techniques using one or more of the following techniques: a bead-based size selection (e.g., AMPure, MagJet, Mag-Bind, Promega Beads, and Kapa Pure Beads), column based PCR cleanup (e.g., Qiagen), or a DNA precipitation based technique such as phenol chloroform or ethanol.

### Sequencing of Cell Barcoded Libraries (e.g., Amplicon Products Containing Cell Barcodes)

Following isolation of the cell barcoded libraries, sequencing can be performed. Sequencing occurs after a purification step; after the purification and additional ligation/PCR steps; or after the purification and additional ligation/PCR and hybridization capture steps.

Any high-throughput technique for sequencing can be used in the practice of the methods described herein. For example, DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like. These sequencing approaches can thus be used to sequence target nucleic acids of interest, for example, nucleic acids encoding target genes and other phenotypic markers amplified from the cell/nuclei populations.

In some embodiments, sequencing comprises whole genome sequencing. In some embodiments, sequencing comprises droplet- or partition-based sequencing.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456:53-59 (2008) or Complete Genomics sequencing, e.g. Drmanac et al, Science, 327:78-81 (2010)), arrays of wells, which may include bead- or particle-bound templates (such as with 454, e.g. Margulies et al, Nature, 437:376-380 (2005) or Ion Torrent sequencing, U.S. patent publication 2010/0137143 or 2010/0304982), micromachined membranes (such as with SMRT sequencing, e.g. Eid et al, Science, 323:133-138 (2009)), or bead arrays (as with SOLID sequencing or polony sequencing, e.g. Kim et al, Science, 316:1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

In some embodiments, sequencing may be performed using a flow cell. DNA/RNA fragments, which contain adapter molecules on either end, are washed across a flow cell (DNA is first denatured into single stranded DNA). This flow cell contains primers which are complementary to the adapter sequences. The bound DNA/RNA is then amplified repeatedly, using unlabelled nucleotides. This forms clusters of DNA/RNA which help produce an amplified signal during sequencing. During sequencing, primers and 4 different fluorescently labelled (reversible) terminator nucleotides are added. Each time a fluorescently labelled nucleotide is incorporated, the label is excited and the fluorescence detected by a camera. The fluorescently labelled terminator can then be removed and the process can continue to sequence the whole fragment. In some embodiments, sequencing is performed on the Illumina® MiSeq platform, (see, e.g., Shen et al. (2012) BMC Bioinformatics 13:160; Junemann et al. (2013) Nat. Biotechnol. 31(4): 294-296; Glenn (2011) Mol. Ecol. Resour. 11(5): 759-769; Thudi et al. (2012) Brief Funct. Genomics 11(1): 3-11; herein incorporated by reference in its entirety), NovaSeq, NextSeq, HiSeq, and the like.

In some embodiments, sequencing may be performed using a nanopore system, in which DNA/RNA molecules pass through a transmembrane protein (e.g. alpha hemolysin or MspA), with different nucleotides providing a different detectable signal as they pass through the channel. For instance, sequencing is performed on the Oxford Nanopore platform (see, e.g., Lu et al (2016), Genomics, Proteomics and Bioinformatics 14:5, herein incorporated by reference in its entirety).

In some embodiments, sequencing may be performed using a solid state nanopore system, in which DNA molecules pass through pores in a metal substrate, with different nucleotides providing a different detectable signal as they pass through the pores.

In some embodiments, sequencing may be performed through utilizing a single circular strand of DNA/RNA. This is created through the ligation of adapters to both ends of a template DNA/RNA molecule. This would then be loaded onto a sequencing unit which provides the smallest available volume for light detection. A single polymerase would be immobilised to the bottom of the base, and replication would begin. The polymerase would use 4 differently labelled nucleotides as a substrate. This would produce a small light pulse with each nucleotide addition which allows identification of the base. This sequencing protocol would produce a movie of light pulses allowing sequencing of the template. In some embodiments, sequencing is performed on the PacBio platform (see, e.g., Rhoads and Au (2015), Genomics, Proteomics and Bioinformatics 13:5, herein incorporated by reference in its entirety).

In some embodiments, sequencing is performed on any preferred, standard sequencing platform.

### Sequencing Analysis

Identification of UWI and association with Cell Barcode

Typical sample barcodes are attached to each genomic fragment produced during library prep and can be sequenced in a variety of methods depending on attachment method and sequencing chemistry. However, in all cases these sample barcodes can be directly associated with the genomic fragment. UWIs are not typical sample barcodes, because they are not directly attached to the genomic fragments. Association of a UWI with a genomic fragment requires additional information provided through the cell barcode. The same cell barcode is attached to UWI fragments and genomic fragments from the same cell. Amplification of these UWI fragments occur in the same way as genomic fragments, with the cell barcoding primer provided according to the single cell technology used. In the case of the 10X single cell ATACseq library prep kit, this includes amplifying the cell barcode on to the UWI via isothermal amplification. After breaking the emulsion, libraries are further amplified with a sample barcode primer hybridizing to the reverse complement of R2 (produced by cell barcoding) and a P5 primer. In the case of single cell amplification method described in PCT/US2023/062776, amplified cell barcoding oligos is used to amplify both the UWI fragment and genomic fragments. Barcodes from both sets of data will be used to cluster cell reads and then UWI information can group the cells together.

The UWI reads can be identified through the UWI sequence and any backbone sequence included in the fragment, and then because the sequenced UWI fragment will contain a cell barcode, a map between UWI and cell barcode can be made linking corresponding genomic fragments to their respective wells.

This method can be adapted to other single cell technologies, including methods described in PCT/US2023/062776, by adapting UWI fragment composition to match requirements for each individual technology.

### Samples/Cells

The sample comprising cells are obtained from cell culture, liquid biopsy, tissue sample. In some embodiments, the sample comprises live cells. In some embodiments, the sample comprises fixed cells. In some embodiments, the sample is a liquid biopsy. In certain embodiments, the sample is a blood sample or a serum sample. In certain embodiments, the sample is a cell suspension obtained from a liquid biopsy. In some embodiments, the sample is a tissue sample. In certain embodiments, the sample is a cell suspension obtained from a tissue sample. In some embodiments, the sample is a cell culture sample. In certain embodiments, the sample is a cell suspension obtained from a cell culture sample.

In some embodiments, the cell is a eukaryotic cell. In some embodiments, the cell is a prokaryotic cells.

In some embodiments, the cells are from a tumor biopsy. In some embodiments, the tumor sample is a solid tumor sample. In some embodiments, the tumor biopsy is a liquid tumor sample. In some embodiments, a tumor sample can include a heterogenous cell population. In some embodiments, the tumor sample is from human tumors such as, but not limited to, tumors from the breast, ovary, lung, prostate, colon, kidney, liver, skin, blood, bone marrow, lymph nodes, spleen, thymus, heart, brain, bladder, adrenal gland, cervix, intestine, pancreas, stomach, smooth muscle, skeletal muscle, thyroid, thymus, endometrium, vulva, etc. In some embodiments, cancer cells that can be detected by the methods of the present disclosure include, but are not limited to, cancer cells from hematological cancers, including leukemia, acute lymphoblastic leukemia, acute myeloid leukemia, acute promyelocytic leukemia, chronic lymphocytic leukemia, chronic myeloid leukemia, childhood leukemia, lymphoma, Hodgkin lymphoma, diffuse large B-cell lymphoma, follicular lymphoma, Burkitt lymphoma, Waldenstrom's macroglobulinemia, non-Hodgkin lymphoma, myeloma myelodysplastic syndromes, polycythemia vera, essential thrombocythemia, myclofibrosis, monoclonal gammopathy of undetermined significance, myeloproliferative neoplasms, amyloidosis, and aplastic anemia. In some embodiments, cancer cells that can be detected by the methods of the present disclosure include, but are not limited to, solid cancers, including for example tumors of the brain (glioblastomas, medulloblastoma, astrocytoma, oligodendroglioma, ependymomas, acoustic neuroma, astrocytoma and glioblastoma, craniopharyngioma, embryonal tumors, glioma, hemangioblastoma, lymphoma of the brain or spinal cord, meningioma, pineal region tumors, pituitary tumors, spinal cord tumors, and Vestibular Shwannoma). In some embodiments, cancer cells that can be detected by the methods of the present disclosure include, but are not limited to, carcinomas, e.g. carcinoma of the lung, liver, thyroid, bone, adrenal, spleen, kidney, lymph node, small intestine, pancreas, colon, stomach, breast, endometrium, prostate, testicle, ovary, skin, head and neck, and esophagus. In some embodiments, cancer cells that can be detected by the methods of the present disclosure include, but are not limited to, small cell lung cancer, combined small cell carcinoma non-small cell lung cancer, adenocarcinoma, squamous cell cancer, large cell carcinoma, salivary gland type tumors, lung sarcoma, lung lymphoma, lung carcinoid tumors, adenoid cystic carcinomas, mesothelioma, and thymomas. In some embodiments, cancer cells that can be detected by the methods of the present disclosure include, but are not limited to, hepatocellular carcinoma, fibrolamellar carcinoma, bile duct cancer, angiosarcoma, and hepatoblastoma. In some embodiments, cancer cells that can be detected by the methods of the present disclosure include, but are not limited to, papillary thyroid cancer, follicular thyroid cancer, anaplastic thyroid cancer, medullary thyroid cancer, thyroid lymphoma, and thyroid sarcoma. In some embodiments, cancer cells that can be detected by the methods of the present disclosure include, but are not limited to osteosarcoma, Ewing tumor, chondrosarcoma, dedifferentiated chondrosarcoma, mesenchymal chondrosarcoma, clear cell, chondrosarcoma, fibrosarcoma, giant cell tumor, and chordoma. In some embodiments, cancer cells that can be detected by the methods of the present disclosure include, but are not limited to, adenoma, adrenocortical carcinoma, neuroblastoma, and pheochromocytoma. In some embodiments, cancer cells that can be detected by the methods of the present disclosure include, but are not limited to, Hemangiosarcoma, and littoral cell angiosarcoma. In some embodiments, cancer cells that can be detected by the methods of the present disclosure include, but are not limited to, renal cell cancer, renal clear cell cancer, renal papillary cancer, chromophobe renal cell cancer, collecting duct carcinoma, renal medullary carcinoma, sarcomatoid type kidney cancer. In some embodiments, cancer cells that can be detected by the methods of the present disclosure include, but are not limited to, adult soft tissue sarcoma, childhood soft tissue sarcoma, neuroendocrine tumors, paraganglioma, intestinal lymphoma, gastrointestinal carcinoid tumors, and gastrointestinal stromal tumors. In some embodiments, cancer cells that can be detected by the methods of the present disclosure include, but are not limited to, adenocarcinoma, ductal adenocarcinoma, cystic tumors, cancer of the acinar cells, endocrine pancreatic tumors, pancreatoblastoma, sarcomas of the pancreas, and pancreatic lymphomas. In some embodiments, cancer cells that can be detected by the methods of the present disclosure include, but are not limited to, small bowel cancer, colon cancer, rectal cancer, anal cancer, squamous cell bowel cancer, carcinoid bowel tumors, bowel sarcomas, bowel lymphoma, and bowel melanomas. In some embodiments, cancer cells that can be detected by the methods of the present disclosure include, but are not limited to, stomach adenocarcinoma, soft tissure stomach sarcomas, gastrointestinal stromal tumors, stomach lymphomas, mucosa associated lymphoid tissue lymphomas, stomach carcinoid tumors. In some embodiments, cancer cells that can be detected by the methods of the present disclosure include, but are not limited to, invasive breast cancer, invasive lobular breast cancer, triple negative breast cancer, inflammatory breast cancer, angiosarcoma of the breast, ductal carcinoma in situ, lobular carcinoma in situ, medullary breast cancer, mucinous breast cancer, tubular breast cancer, adenoid cystic carcinoma of the breast, metaplastic breast cancer, lymphoma of the breast, basal type breast cancer, phyllodes, cystosarcoma phyllodes, papillary breast cancer, and Paget's disease of the breast. In some embodiments, cancer cells that can be detected by the methods of the present disclosure include, but are not limited to, endometroid adenocarcinoma, uterine serous carcinoma, and clear cell carcinoma of the endometrium. In some embodiments, cancer cells that can be detected by the methods of the present disclosure include, but are not limited to, acinar adenocarcinoma of the prostate, ductal adenocarcinoma of the prostate, transitional cell cancer of the prostate, squamous cell cancer of the prostate, small cell prostate cancer, neuroendocrine tumors of the prostate, and sarcomas of the prostate. In some embodiments, cancer cells that can be detected by the methods of the present disclosure include, but are not limited to, seminomas, classic seminomas, spermatocytic seminomas, non-seminomatous germ cell tumors, embryonal carcinoma, yolk sac carcinoma, choriocarcinoma teratomas of the testicles, Leydig cell tumors, and Sertoli cell tumors. In some embodiments, cancer cells that can be detected by the methods of the present disclosure include, but are not limited to, epithelial ovarian cancer, germ call ovarian tumors, sex cord stromal tumors, and borderline ovarian tumors. In some embodiments, cancer cells that can be detected by the methods of the present disclosure include, but are not limited to, basal cell carcinoma of the skin, melanoma, non-melanoma skin cancer, Merkel cell cancer, cutaneous skin lymphomas, Kaposi sarcoma, skin adnexal tumors, skin sarcomas, and squamous cell carcinoma of the skin. In some embodiments, cancer cells that can be detected by the methods of the present disclosure include, but are not limited to, oropharyngeal cancer, hypopharyngeal cancer, laryngeal cancer, lip and oral cavity cancer, nasopharyngeal cancer, paranasai sinus and nasal cavity cancer, salivary gland cancer, squamous cell neck cancer, and soft tissue sarcoma. In some embodiments, cancer cells that can be detected by the methods of the present disclosure include, but are not limited to, esophageal adenocarcinoma, esophageal squamous cell carcinomas, esophageal small cell carcinoma. In some embodiments, cancer cells that can be detected by the methods of the present disclosure include, but are not limited to, secondary cancers caused by metastasis.

Tumor microenvironments contain a heterogenous population of cells. Characterizing the composition and the interaction, dynamics, and function of a heterogenous population of cells at the single-cell resolution are important for fully understanding the biology of tumor heterogeneity, under both normal and diseased conditions. For example, cancer, a disease caused by somatic mutations conferring uncontrolled proliferation and invasiveness, can benefit from advances in single-cell analysis. Cancer cells can manifest resistance to various therapeutic drugs through cellular heterogeneity and plasticity. The tumor microenvironment includes an environment containing tumor cells that cooperate with other tumor cells and host cells in their microenvironment and can adapt and evolve to changing conditions.

In some embodiments, the heterogeneous population of cells can include, but are not limited to, inflammatory cells, cells that secret cytokines and/or chemokines, cytotoxic immune cells (e.g., natural killer cells, natural killer T cells, and/or CD8+ T cells), immune cells, macrophages (e.g., immunosuppressive macrophages, tumor-associated macrophages, classically activated (M1) macrophages, alternatively-activated (M4) macrophages, and/or all macrophage subtypes including: M4, Mox, Mhem, M(Hb), M2a/2b/2c, and M1), antigen-presenting cells, cancer cells, tumor-associated neutrophils, erythrocytes, dendritic cells (e.g., myeloid dendritic cells, plasmacytoid dendritic cells, langerhans cells, and/or interdigitating dendritic cells), eosinophils (e.g. eotaxin-1 (CCL-11), eotaxin-2 (CCL-24), and eotaxin-3 (CCL-26)), mast cells (e.g. mucosal mast cells and connective tissue mast cells), T helper cells (e.g. CD4+ T cells, Th1 cells, Th2 cells, Th3 cells, Th17 cells, and TFH cells), regulatory T cells (e.g. natural T regularoty cells and induced regulatory T cells), memory T cells (e.g. central memory T cells and effector memory T cells), B cells (e.g. transitional, naïve, plasma, and/or memory cells), tumor-infiltrated T cells, fibroblasts, endothelial cells (e.g. vascular endothelial cells and/or lymphatic endothelial cells), PD1+ T cells, and the like.

In some embodiments, the cell population comprises neuronal cells. Non-limiting examples of neuronal cells include neurons (e.g. motor neurons, sensory neurons, intermediary nerons, and relay neurons), astrocytes, oligodendrocytes, microglia, ependymal cells, satellite cells, and schwann cells.

In some embodiments, the cell population comprises cardiac cells. Non-limiting examples of cardiac cells include cardiac fibroblast cells, cardiomyocytes, smooth muscle cells, and endothelial cells.

In some embodiments, the cell population comprises fibroblast cells.

In some embodiments, the sample can be from cell lines such as ovarian cancer (e.g. A4, OVCAR3, CAOV3, CAOV4, ES-2, OV-90, TOV-112D, TOV-21G, UWB1.289, UWB1.289+BRCA1, 59M, A2780, A2780CIS, A2780ADR, COLO720E, COV318, COV362, COV362.4, COV413A, COV413B, COV504, COV644, OAW28, OAW42, OV56, OV7, OV17R, PEA1, PEA2, PEO1, PEO4, PEO14, PEO16, PEO23, SKOV3, ECACC, 2774, A2780, HOC7, SKOV3, SKOV6, IGROV1), teratocarcinoma (e.g. NT2, P19, F9), colon cancer (e.g. HT29, CL40, SW1417, CW2), prostate (e.g. PC3, DU145, LNCaP), cervical cancer (e.g. C33A, HT-3, ME180), kidney cancer (e.g. ACHN, A-498, 786-O, Caki-1, Caki-2, 769-P, RCC4, SMKT-R), lung cancer (e.g. A549, PC9, NCIH-322, SHP-77, CORL23, NCIH727, NCI-H358), skin cancer (e.g. A431, A375, HS695T, HS1CLS, IGR1, MELCSLI, MEWO, MML1, NISG, SKMEL1, WSICLS), glioma (e.g. C6, LN229, SNB19, U87, U251), but are not limited to only these lines.

In some embodiments, the cell population comprises animal cells (in particular, non-human animal cells). In some embodiments, the cells include, but are not limited to, non-human mammalian cells, guinea pig cells, rabbit cells, hamster cells, non-human primate cells, dog cells, pig cells, domestic cat cells, sheep cells, mice cells, rat cells, bird cells, amphibian cells, reptile cells, fish cells (e.g. zebra fish cells), cattle cells, chicken cells, goat cells, turkey cells, and horse cells.

In some embodiments, the cell population comprises invertebrate animal cells, such as insect cells e.g. cells from Drosophila.

In some embodiments, the cell population comprises primary cells from these animals. In some embodiments, the cell population comprises cell lines derived from these animals. Non-limiting examples of cell lines include cell lines from, Spodoptera frugiperda (e.g. Sf9 cells), Trichoplusia ni (e.g. Tni-FNL cells), Drosophila melanogaster (e.g. S2, S2R+, S2-DGRC, and Kc167 cells), Heliothis virescens (e.g. BCIRL-Hz-AM1 and FB33 cells), the mosquito (e.g. Aag2 and A20), Hamster (e.g. Chinese hamster ovary (CHO) cells), mouse (e.g. 3T3-L1, ALC, and bEnd.3 cells), Rat (e.g. 9L and B35 cells), Zebrafish (e.g. AB9 cells), Dog (e.g. CMT12 and D17 cells), African green monkey (e.g. MA-104 and Vero cells), and Cercopithecus aethiops (e.g. Cos-7 cells).

In some embodiments, the cell population comprises cells which have been genetically modified. In some embodiments, the cells have been genetically modified in-vitro. In some embodiments, the cells comprise of cells derived from human patients and/or animals who have undergone gene therapy e.g. faulty/inactive gene replacement, and introduction of a new gene to a cell(s). In some embodiments, the cells may comprise of genetically modified immune cells. A non-limiting example of a genetically modified immune cell is a chimeric-antigen receptor T cells (CAR-T cells). In some embodiments, the cells have been genetically modified through genome editing technology. Examples of genome editing technology which can be used to genetically modify cells include, but are not limited to, CRISPR (e.g. CRISPR/Cas9), transcription activator-like effector nucleases (TALENs), zinc-finger nucleases (ZFNs), and homing endonucleases/meganucleases.

In some embodiments, the cell population comprises plant cells. In some embodiments, the cells comprise of plant cells from the following plant species: Arabidopsis thaliana, Boechera spp, Salaginella moellendorffi, Brachypodium distachyon, Setaria viridis, Lotus japonicus, Lemna gibba, Zea mays, Medicago truncatula, Mimulus guttatus, Nicotania benthamiana, Nicotania tabacum, Oryza sativa, Physcomitrella patens, Marchantia polymorpha, Populus spp, Chlamydomonas reinhardtii, Beta vulgaris, Agrostis canina, Agrostis gigantea, Agrostis stolonifera, Agrostis capillaris, Alopecurus pratensis, Arrhenatherum elatius, Bromus catharticus, Bromus sitchensis, Cynodon dactylon, Dactylis glomerata, Festuca arundinacea, Festuca filiformis, Festuca ovina, Festuca pratensis, Festuca rubra, Festuca trachyphylla, Festulolium Asch, Lolium multiflorum, Lolium perenne, Phalaris aquatica, Phleum nodosum, Phleum pratense, Poa annua, Poa nemoralis, Poa palustris, Poa pratensis, Poa trivialis, Trisetum flavescens, Biserrula pelecinus, Galega orientalis, Hedysarum coronarium, Lotus corniculatus, Lupinus albus, Lupinus angustifolius, Lupinus luteus, Medicago italica, Medicago littoralis, Medicago lupulina, Medicago polymorpha, Medicago rugosa, Medicago sativa, Medicago scutellate, Medicago truncatula, Onobrychis viciifolia, Ornithopus sativus, Trifolium alexandrinum, Trifolium fragiferum, Trifolium glanduliferum, Trifolium hirtum, Trifolium hybridum, Trifolium incarnatum, Trifolium isthmocarpum, Trifolium michelianum, Trifolium pratense, Trifolium repens, Trifolium resupinatum, Trifolium squarrosum, Trifolium subterraneum, Trifolium vesiculosum, Trigonella foenum-graecum, Vicia benghalensis, Vicia faba, Vicia pannonica, Vicia sativa, Vicia villosa, Brassica napus, Brassica oleracea, Phacelia tanacctifolia, Plantago lanccolata, Raphanus sativus, Arachis hypogca, Brassica rapa, Brassica juncea, Brassica napus, Brassica nigra, Cannabis sativa, Carthamus tinctorius, Gossypium spp, Helianthus annuus, Linum usitatissimum, Papaver somniferum, Sinapis alba, Glycine max, Avena nuda, Avena sativa, Avena strigose, Hordeum vulgare, Phalaris canariensis, Sorghum bicolor, Sorghum sudanense, Sorghum bicolor, Triticosecale Wittm, Triticum durum, Triticum spelta, and Solanum tuberosum.

In some embodiments, the cell population comprise of the following types of plant cells: parenchyma cells, palisade parenchyma cells, ray parenchyma cells, collenchyma cells, angular collenchyma cells, annular collenchyma cells, lamellar collenchyma cells, lacunar collenchyma cells, sclerenchyma cells, fibre sclerenchyma cells, sclereid sclerenchyma cells, xylem cells, phloem cells, sieve tube member cells, companion cells, sieve cells, meristematic cells, apical meristem cells, lateral meristem cells, intercalary meristem cells, epidermal cells, pavement cells, stomatal guard cells, and trichomes cells.

In some embodiments, the cell population comprise of prokaryotic organisms. In some embodiments, the prokaryotic organisms comprise bacterial cells. In certain embodiments, the bacterial cells comprise gram negative bacteria or gram positive bacteria. Non-limiting examples of bacterial cells include Enterobacteriaceae, such as Salmonella and Escherichia, Caulobacter, myxococcus, streptomyces, bacillus, clostridium, Bifidobacterium, Helicobacter pylori, Staphylococcus, and Streptococcus.

In some embodiments, the cell population comprises bacterial cells. Examples of bacterial cells include, but are not limited to, Acetobacter aurantius, Acinetobacter baumannii, Actinomyces israelii, Agrobacterium radiobacter, Agrobacterium tumefaciens, Anaplasma spp, Anaplasma phagocytophilum, Azorhizobium caulinodans, Azotobacter vinelandii, viridans streptococci, Bacillus spp, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides spp, Bacteroides fragilis, Bacteroides gingivalis, Bacteroides melaninogenicus (now known as Prevotella melaninogenica), Bartonella, Bartonella hensclac, Bartonella quintana, Bordetella, Bordetella bronchiseptica, Bordetella pertussis, Borrelia burgdorferi, Brucella spp, Brucella abortus, Brucella melitensis, Brucella suis, Burkholderia spp, Burkholderia mallei, Burkholderia pseudomallei, Burkholderia cepacia, Calymmatobacterium granulomatis, Campylobacter spp, Campylobacter coli, Campylobacter fetus, Campylobacter jejuni, Campylobacter pylori, Chlamydia, Chlamydia trachomatis, Chlamydophila spp, Chlamydophila pneumoniae, Chlamydophila psittaci, Clostridium spp, Clostridium botulinum, Clostridium difficile, Clostridium perfringens, Clostridium tetani, Corynebacterium spp, Corynebacterium diphtheriae, Corynebacterium fusiforme, Coxiella burnetii, Ehrlichia chaffeensis, Ehrlichia ewingii, Eikenella corrodens, Enterobacter cloacae, Enterococcus spp, Enterococcus avium, Enterococcus durans, Enterococcus faecalis, Enterococcus faecium, Enterococcus gallinarum, Enterococcus maloratus, Escherichia coli, Fusobacterium necrophorum, Fusobacterium nucleatum, Gardnerella vaginalis, Haemophilus spp, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus spp, Lactobacillus acidophilus, Lactobacillus bulgaricus, Lactobacillus casei, Lactococcus lactis, Legionella pneumophila, Leptospira interrogans, Leptospira noguchii, Listeria monocytogenes, Methanobacterium extroquens, Microbacterium multiforme, Micrococcus luteus, Moraxella catarrhalis, Mycobacterium spp, Mycobacterium avium, Mycobacterium bovis, Mycobacterium diphtheriac, Mycobacterium intracellulare, Mycobacterium leprae, Mycobacterium lepraemurium, Mycobacterium phlei, Mycobacterium smegmatis, Mycobacterium tuberculosis, Mycoplasma spp, Mycoplasma fermentans, Mycoplasma genitalium, Mycoplasma hominis, Mycoplasma penetrans, Mycoplasma pneumoniae, Mycoplasma mexican, Neisseria, Neisseria gonorrhoeae, Neisseria meningitidis, Pasteurella spp, Pasteurella multocida, Pasteurella tularensis, Peptostreptococcus, Porphyromonas gingivalis, Prevotella melaninogenica, Pseudomonas aeruginosa, Rhizobium radiobacter, Rickettsia spp, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella spp, Salmonella enteritidis, Salmonella typhi, Salmonella typhimurium, Serratia marcescens, Shigella dysenteriae, Spirillum volutans, Staphylococcus spp, Staphylococcus aureus, Staphylococcus epidermidis, Stenotrophomonas maltophilia, Streptococcus spp, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema spp, Ureaplasma urealyticum, Vibrio spp, Vibrio cholerae, Vibrio comma, Vibrio parahaemolyticus, Vibrio vulnificus, Wolbachia spp, Yersinia spp, Yersinia enterocolitica, Yersinia pestis, Yersinia pseudotuberculosis.

In some embodiments, the cells comprise of simple eukaryotic organisms. Non-limiting examples of a simple eukaryotic organisms are yeasts, such as Saccharomyces (e.g. Saccharomyces cerevisae), Schizosaccharomyces Candida, or Pichia; also Euglenophyta, Chlorophyta (green algae), Diatoms, Dinoflagellate Euglenophyta, Chlorophyta, Diatoms, and Dinoflagellate. In some embodiments, the eukaryotic organisms are fungi cells. In some embodiments, the eukaryotic organisms are plant cells. In some embodiments, the eukaryotic organisms are mammalian cells.

In some embodiments, the cell population comprise non-human cells. In some embodiments, the cell population comprise human cells. In some embodiments, the cell population comprise rodent cells. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell. In certain embodiments, the cell population is a mixture of one or more cells selected from: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the cell populations within the sample are from mutated/malignant tissue or abnormal blood. In some embodiments, the methods of the present disclosure steps are also performed on cell populations within the sample that are from non-mutated/benign tissue or normal blood, which serve as a controls sample. In some embodiments, the cell populations within the sample are from both non-mutated tissue or normal blood, which serves as a “tumor-normal” control sample, and mutated/malignant tissue and abnormal blood, which serves as a “target” sample. For example, aspects of the present methods also include performing tumor normal analysis from normal cells within a biopsy, e.g., for example where the “target” sample came from. Such methods allow for detecting and diagnosing cell populations from non-mutated tissue or normal blood to determine if mutations are found in familial germlines that may also develop in other places of the body, or if the mutations are somatic to provide for better treatment options.

In various embodiments, the sample comprises no more than 100k cells, such as no more than 90k, no more than 80k, no more than 70k, no more than 60k, no more than 50k, no more than 40k, no more than 30k, no more than 20k, no more than 15k, no more than 10k cells, no more than 5k, no more than 1k, no more than 500, no more than 200, or no more than 100 cells. In certain embodiments, the sample comprises about 50k cells. In certain embodiments, the sample comprises about 50k cells. In certain embodiments, the sample comprises about 40k cells. In certain embodiments, the sample comprises about 30k cells. In certain embodiments, the sample comprises about 20k cells. In certain embodiments, the sample comprises about 15k cells. In certain embodiments, the sample comprises about 10k cells. In certain embodiments, the sample comprises about 5k cells. In certain embodiments, the sample comprises about 1k cells. In specific embodiments, the sample comprises about 500 cells. In specific embodiments, the sample comprises about 200 cells. In specific embodiments, the sample comprises about 100 cells.

### NON-LIMITING EXEMPLARY EMBODIMENTS OF IN PARTITION AMPLIFICATION

### Embodiment 3. In Partition Amplification of Cell Barcode Primer for Combinatorial Cell Barcoding Single Cell Prep

This disclosure also features methods to facilitate cell identification when multiple cell barcodes enter a partition (e.g., a droplet prepared by the digital PCR instrument) where the cell barcode entities (see, e.g., Embodiment 1 reagent (2)) comprise a single cell barcoding oligo that is amplified in the partition to create a clonal population of the single cell barcoding primer having complementarity to UniversalSequence1. Clonal population is defined as multiple identical copies of a barcode sequence, allowing for synthesis error. Traditional barcoded beads include clonal barcodes on each of the beads. In the present method, amplification of a single sequence inside of a cell/partition also results in a clonal population. For example, if two sequences are inside of a cell/partition then two clonal populations are made in the cell.

Non-limiting examples of single cell barcoding oligos include those described in PCT/US2023/062776 (now published as WO 2023/159151), which is hereby incorporated by reference in its entirety.

In some embodiments, the methods described herein include cell barcode entities (e.g., see Embodiment 1 reagent (3)) that comprise a single cell barcoding oligo that is amplified in the partition to create a clonal population of the single cell barcoding primer having complementarity to UniversalSequence2. Non-limiting examples of single cell barcoding oligos include those described in PCT/US2023/062776 (now published as WO 2023/159151), which is hereby incorporated by reference in its entirety.

In some embodiments, the methods described herein include an additional component comprising a barcoding oligo amplification primer. Non-limiting examples of barcoding amplification primers include those described in PCT/US2023/062776 (now published as WO 2023/159151), which is hereby incorporated by reference in its entirety. In such cases, an additional step involving isothermal amplification of the barcoding oligo using the barcoding oligo amplification primer occurs before amplification of the library.

In some embodiments, the in-partition amplification is used performed in a digital PCR instrument using digital PCR workflow or digital PCR workflow adapted to include the described steps.

### Embodiment 4. In Partition Amplification of Cell Barcode Primer Double Poisson Single Cell Prep

This disclosure also features methods to facilitate cell identification when multiple cell barcodes enter a partition (e.g., a droplet prepare by the digital PCR instrument) where the cell barcode entities (see, e.g., Embodiment 1 reagent (2) comprises a single cell barcoding oligo that is amplified in the partition to create a clonal population of the single cell barcoding primer having complementarity to UniversalSequence1 or UniversalSequence2. Non-limiting examples of single cell barcoding oligos include those described in PCT/US2023/062776 (now published as WO 2023/159151), which is hereby incorporated by reference in its entirety.

In some embodiments, the methods described herein include an additional component comprising a barcoding oligo amplification primer. Non-limiting examples of barcoding amplification primers include those described in PCT/US2023/062776 (now published as WO 2023/159151), which is hereby incorporated by reference in its entirety. In such cases, an additional step involving isothermal amplification of the barcoding oligo using the barcoding oligo amplification primer occurs before amplification of the library.

In some embodiments, the in-partition amplification is performed in a dPCR instrument using dPCR workflow or dPCR workflow adapted to include the described steps. In some embodiments, the in-partition amplification is performed in an instrument selected from: Geode (Stilla technologies), Chromium (10X Genomics), ddSEQ Single-Cell Isolator (Biorad), QX200 Droplet Digital PCR System (Biorad), QX600 Droplet Digital PCR System (Biorad), QX One Droplet Digital PCR System (Biorad), Qiacuity Digital PCR Machine (Qiagen), QuantStudio Absolute Q Digital PCR System (ThermoFisher), HIVE CLX (Honeycomb), BD Rhapsody (BD Biosciences), Icell8 (Takara), Asteria (Scipio), Pipseq (Fluent), Biomark X9 System (Standard Biotools), C1 System (Standard Biotools), SH800S Cell Sorter (Sony Biotechnology), BD FACS Melody (BD Biosciences), CytoFlex SRT Benchtop Cell Sorter (Beckman Coulter), and MoFlo Astrios Cell Sorter (Beckman Coulter).

### Embodiment 5: Pre-Preparation of In Situ Ligation Libraries Before Droplet-Based Isolation

This disclosure features methods where pre-cursor ligation libraries can be prepared in situ within a bulk reaction before partitioning the cells in a droplet. Note that the sample pooling methods described above may be applied prior to creating pre-cursor ligation libraries.

Each cell droplet can then be merged with zero, one, or more additional droplets such that the droplet is capable of labeling the cell with a unique cell barcode or barcode combination.

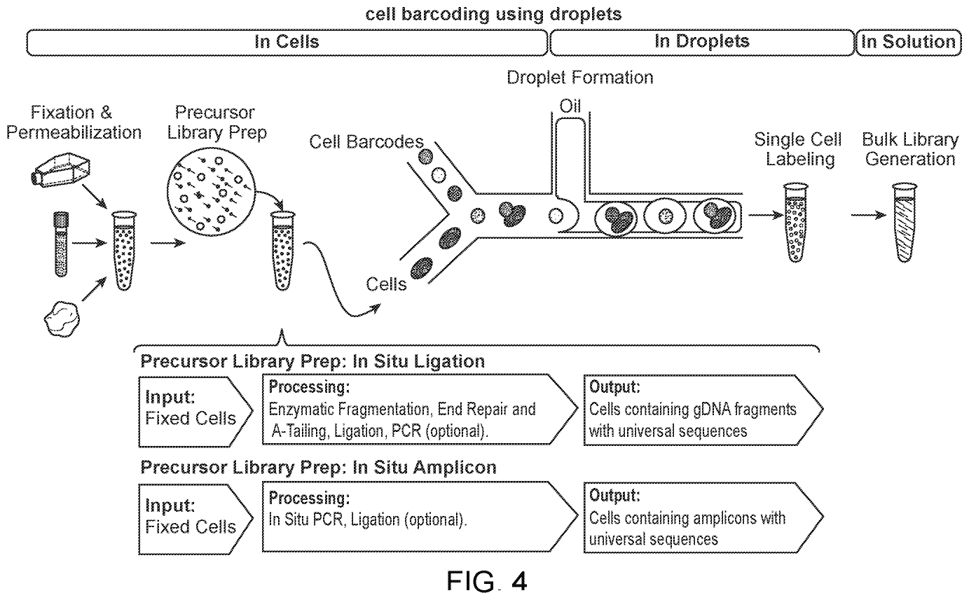

In one embodiment, the method includes:
Step 1: performing a bulk in situ reaction according to equivalent steps in PCT/US2021/046025 and PCT/US2023/062776, each of which are hereby incorporated by reference in their entireties. An example of partitioning precursor libraries in a droplet is shown in FIG. 4. As shown in FIG. 4, precursor libraries prepared using in situ library preparation methods are then subjected to cell barcodes using droplets, allowing for single cell labeling and bulk library generation.
Step 1a. Fixing and Permeabilizing;
Step 1b. Fragmentating and End Repair A-tailing;
Step 1c. Ligating (in situ buffer exchange optional before and after ligation step);
Step 2: Partitioning the cell into a droplet;
Step 3: Merging cell droplet with a droplet containing indexing primers;
Step 4: Performing PCR; and
Step 5: Isolating libraries from droplets in which PCR was performed.

In some embodiments, the method includes adding an additional protease step to aid library amplification in the droplet.

In some embodiments, cell droplets can be formed (step 2) in a buffer containing all PCR reagents, except for the indexing primers. In some embodiments, cell droplets can be formed (step 2) in a buffer containing all PCR reagents, including indexing primers. In some embodiments, indexing primers can be added to the pre-droplet cell buffer on beads. In some embodiments, a precursor molecule to the indexing primer can be added to the pre-droplet cell buffer, this molecule will be amplified after droplet formation. In some embodiments, a PCR reaction can occur within the cells, but before forming a cell droplet. In some embodiments, Step 1 is not ligation library prep, but another method for creating pre-cursor libraries within cells ((i.e., in situ PCR amplification, or RNA amplification, tagmentation)).

In some embodiments, cell droplets can be formed (step 2) in a carrier fluid, such as PBS or buffers standardly used in the industry.

In some embodiments, cell droplets can be merged (step 3) with one or more different types of droplet in a sequential manner.

In some embodiments, indexing primers can be in droplets on beads (step 3).

In some embodiments, PCR reagents can also be included in the indexing primer droplet (step 3).

In some embodiments, indexing primers can be in droplets not on beads (step 3), such droplets could have been formed in absence of beads, or by cleaving the primers off of the beads.

In some embodiments, indexing primers in droplets can be generated from indexing oligos by primer invasion PCR or alternative isothermal amplification methods, for example, as described in PCT/US2022/019845, which is hereby incorporated by reference in its entirety.

In some embodiments, indexing primer droplets do not contain the indexing primer, but contain a precursor molecule to make the indexing primer (e.g., an indexing oligo). For example, the indexing primers are generated from the indexing oligo using primer invasion PCR or alternative isothermal amplification methods, for example, as described in PCT/US2022/019845, which is hereby incorporated by reference in its entirety.

In some embodiments, performing PCR (step 4), includes performing thermocycling conditions to allow indexing primers to amplify the pre-cursor libraries.

In some embodiments, performing PCR (step 4), can include performing isothermal amplification to amplify indexing oligos preparing the indexing primer in the merged cell droplet.

In some embodiments, performing PCR (step 4) can include merging a third droplet with the cell droplet after isothermal amplification is performed.

In some embodiments, the method includes optimization of the isolating libraries from droplets in which PCR was performed (step 5).

### Embodiment 6: Methods of Performing Library Partition and Droplet-Based Sequencing with Cells Conjugated to Magnetic Beads

The present disclosure also includes using the magnetic beads conjugated cells in methods where pre-cursor ligation libraries can be prepared in situ within a bulk reaction before partitioning the cells in a partition. Each cell droplet can then be merged with zero, one, or more additional droplets such that the droplet is capable of labeling the cell with a unique cell barcode or barcode combination.

### Embodiment 7: Methods of Bead Conjugation Plus Preparing an In Situ Library for Sequencing

Provided herein are methods of preparing an in situ library for sequencing with magnetic beads. The methods comprise: (a) mixing a sample comprising cells with magnetic beads; (b) incubating the mixture; (c) adding a fixing agent; (d) washing the cells fixed to the magnetic beads by magnetic pelleting; (e) attaching universal sequences to nucleic acid inside of the cells; and (f) purifying the nucleic acid products. In some embodiments, the methods further comprise a step of adding a quenching agent after step (c). In various embodiments, adding the quenching agent improves the fragment distribution of the in situ library.

Also provided herein are methods of preparing an in situ library for sequencing with magnetic beads comprising: (a) incubating a sample comprising cells in the presence of a fixing agent; (b) mixing the fixed cells with magnetic beads; (c) incubating the mixture; (d) washing the cells fixed to the magnetic beads by magnetic pelleting; (e) attaching universal sequences to nucleic acid inside of the cells; and (f) purifying the nucleic acid products. In some embodiments, the methods further comprise a step of adding a quenching agent after step (c). In various embodiments, adding the quenching agent improves the fragment distribution of the in situ library.

The universal sequences can be any sequences, oligonucleotides, or primers described above for the preparation of a ligation-based, or amplicon-based in situ library. It can also include barcodes. In some embodiments, the universal sequences are adapter oligonucleotides. In some embodiments, the universal sequences are barcoding oligonucleotides.

### KITS

Aspects of the present disclosure include kits for preparing in situ precursor libraries and cell barcoded libraries in partitions.

Aspects of the present disclosure provides a kit for preparing, in situ libraries and barcoded libraries from a cell population for analyzing a population of cells. The kit may comprise one or more primer sets, barcoding oligonucleotides, reagents, enzymes, and/or buffers described herein contained in the compositions. The kit may further comprise written instructions for processing and analyzing a heterogeneous population of cells based on the sequencing of the cells and phenotypic markers. The kit may comprise one or more primer sets, oligonucleotides, reagents, enzymes, and/or buffers described herein contained in the compositions. The kit may further comprise written instructions for generating primers from oligonucleotides using linear amplification. The kit may also comprise reagents for performing amplification techniques (e.g., PCR, isothermal amplification, ligation, tagmentation etc.), hybridization capture, purification, and/or sequencing (e.g., Next Generation Sequencing). In some cases, the kit also includes reagents for fragmentation and ligation of consensus regions to a DNA or RNA fragment.

In some embodiments, the kit comprises the components necessary for in situ library preparation of precursor libraries, and components necessary for in situ cell barcoding preparation to produce cell barcoded libraries, in partitions, and optionally components necessary for performing partition-based sequencing.

In some embodiments, the kit comprises one or more microfluidic chips. In some embodiments, the kit comprises one or more instruments for carrying one or more of the steps of the methods presented herein (e.g. partitioning, cell barcoding, sequencing). In some embodiments, instrument is one or more of: Geode (Stilla technologies), Chromium (10X Genomics), ddSEQ Single-Cell Isolator (Biorad), QX200 Droplet Digital PCR System (Biorad), QX600 Droplet Digital PCR System (Biorad), QX One Droplet Digital PCR System (Biorad), Qiacuity Digital PCR Machine (Qiagen), QuantStudio Absolute Q Digital PCR System (ThermoFisher), HIVE CLX (Honeycomb), BD Rhapsody (BD Biosciences), Icell8 (Takara), Asteria (Scipio), Pipseq (Fluent), Biomark X9 System (Standard Biotools), C1 System (Standard Biotools), SH800S Cell Sorter (Sony Biotechnology), BD FACS Melody (BD Biosciences), CytoFlex SRT Benchtop Cell Sorter (Beckman Coulter), and MoFlo Astrios Cell Sorter (Beckman Coulter).

In some embodiments, the kit comprise a fragmentation enzyme and buffer for performing an enzymatic fragmentation reaction to form one or more nucleic acid fragments within a cell; an End repair and A tail (ERA) master mix and buffer for performing an end-repair and A-tailing reaction on the one or more nucleic acid fragments; a ligation enzyme and buffer; adapter sequences, wherein the ligation enzyme and buffer, and adapter sequences are capable of ligating, in each cell, the nucleic acid fragments to the adapter sequences in situ to create a ligated library comprising ligated nucleic acid fragments; amplification primers for amplifying the ligated nucleic acid fragments to form amplicon products; a polymerase chain reaction (PCR) enzyme master mix comprising one or more of: an enzyme, a buffer, or an enzyme and a buffer; in an amount sufficient to prepare a precursor library in situ.

In some embodiments, the kit further comprises cell barcoding oligonucleotides comprising: an oligonucleotide, wherein the oligonucleotide comprises: an amplification sequence, and a consensus region that is complementary to a target sequence of a nucleic acid fragment; and a second oligonucleotide, wherein the second oligonucleotide comprises: a second amplification sequence, a second consensus region that complementary to a target sequence of a nucleic acid fragment; amplification reagents comprising: a first amplification primer comprising a nucleotide sequence that is complementary to the amplification sequence on the oligonucleotide; a second amplification primer comprising a nucleotide sequence that is complementary to the second amplification sequence on the second oligonucleotides; and an isothermal amplification polymerase.

In some embodiments, the kit further comprises instructions for carrying out the precursor library preparation in situ;

In some embodiments, the kit further comprises instructions for preparing a mixture in one or more containers comprising one or more of: the cell population, cell barcoding oligonucleotides, and amplification reagents;

In some embodiments, the kit further comprises instructions for partitioning the mixture into a plurality of partitions.

In some embodiments, the kit further comprises instructions for barcoding the precursor libraries in the plurality of partitions.

In some embodiments, the kit further comprises instructions for isolating the cell barcoded libraries; and

In some embodiments, the kit further comprises instructions for sequencing the cell barcoded libraries.

In some embodiments, the kit comprising instructions for carrying out the precursor library comprises the following steps: performing, in each cell of the cell population, an enzymatic fragmentation reaction to form nucleic acid fragments; ligating, in each cell, the nucleic acid fragments to adapter sequences sequences in situ to create a precursor library comprising ligated nucleic acid fragments.

In some embodiments, the kit comprising instructions for preparing the mixture comprises the following steps: preparing the mixture in one or more containers comprising one or more of: the cell population, cell barcoding oligonucleotides, and amplification reagents

In some embodiments, the kit comprising instructions for partitioning the mixture into a plurality of partitions comprises the following steps: partitioning the mixture into a plurality of partitions, wherein at least some partitions contain: a cell from the cell population comprising the precursor library, and a plurality of cell barcoding oligonucleotides.

In some embodiments, the kit comprising instructions for barcoding the precursor library in a plurality of partitions comprises the following steps: barcoding the precursor libraries in the plurality of partitions by: amplifying the cell barcoding oligonucleotides in the at least some partitions to produce cell barcoding primers; amplifying the precursor libraries in partitions with the cell barcoding primers to produce cell barcoded libraries.

In some embodiments, the kit further comprises one or more buffers.

In some embodiments, the kit further comprises a cell lysis buffer.

In some embodiments, the amplification primers comprise barcoding primers, sequencing primers, or a combination thereof.

In some embodiments, the kit further comprises protease K.

In some embodiments, the kit further comprises barcoding primers, and a second PCR Enzyme master mix comprising one or more of: an enzyme, a buffer, or an enzyme and a buffer.

In some embodiments, the kit further comprises a lytic enzyme.

In some embodiments, a thermocycler.

In some embodiments, a partitioning instrument for partitioning cells, cell barcoding oligonucleotides, or a combination of cells and cell barcoding oligonucleotides.

In some embodiments, an instrument comprising a thermocycler and a partition engine.

In some embodiments, the partition engine is configured to hold one or more microfluidic chips.

In some embodiments, the one or more microfluidic chips is configured to hold a sample comprising one or more cells in the cell population, amplification reagents, and one or more cell barcoding oligonucleotides.

### 6. EXAMPLES

### Example 1: In Situ Library Prep and Cell Barcoding

Experiment 1: First Attempt with in Situ Library Prep (precursor libraries) and Cell Barcoding

To test whether precursor libraries containing mouse and human cells and that were cell barcoded in droplets could be sequenced and analyzed to determine sequencing read contribution of human and mouse cells, GM12878 cells and EL4 cells were fixed according to protocol A, independently.

### Protocol A: Cell Fixation

Cells were resuspended to a concentration of 1M cells/mL. IncellMax (IncellDx) fixative, is added to cells to a final concentration of 1X IncellMax buffer and 0.5 cells/mL. Cells incubate for 1 hour at room temperature with rotation. Centrifuged at 1500×g for 5 minutes. Supernatant is decanted off of cell pellet and cells washed with PBS 1 time. Cells were resuspended in PBS to a final concentration of 1M cells/ml.

The GM12878 cells and EL4 cells were mixed at a ratio of 70% Human to 30% Mouse, before preparing precursor libraries using 100K cells. Precursor libraries were prepared following protocol D, below:

### Protocol D: In Situ Library Prep, Base Protocol.

In situ pre-cursor libraries were prepared following the protocol as described in PCT Patent Application Publication Nos. WO2022036273 and WO2022192603, up through ligation. In brief, cells were fixed using either Protocol A, washed with dPBS at 1500×g, DNA accessibility was performed by incubating cells in dPBS at 95*C for 20 minutes, then placing on ice.

Fragmentation and End Repairs was performed by adding the heated cells to 9 uL of Frag/AT Enzymes and 4 ul of Frag/AT buffer (Watchmaker Genomics) to a final volume of 50 uL.

Cells were then incubated at 37*C for 20 minutes and 65*C for 30 min. 20 uL of Ligation Master Mix (Watchmaker Genomics) and 5 uL of 15 uM Annealed Y-Adapter was added to the reaction.

Ligation was performed at 20*C for 15 minutes. Cells were washed with dPBS by raising the volume to 200 uL, removing all but 2 uL supernatant and then resuspending in 20 uL.

12K cells were then used for single cell generation using protocol F, below:

Protocol F: 10X Cell Barcoding with In Situ Prepped Cells (Precursor Libraries).

In situ library prepped cells from Protocol D were aliquoted to individual reactions in accordance to the 10X Chromium Next GEM Single Cell ATAC Reagent Kits v2 guidelines and concentrated or diluted in dPBS as required to achieve 8 uL of cells. 7 uL of ATAC Buffer B were added to each tube and mixed by pipetting. Steps 2.1 through 4.2 in the 10X Chromium Next GEM Single Cell ATAC Reagent Kits v2 (CG000496 Rev B) were performed as written.

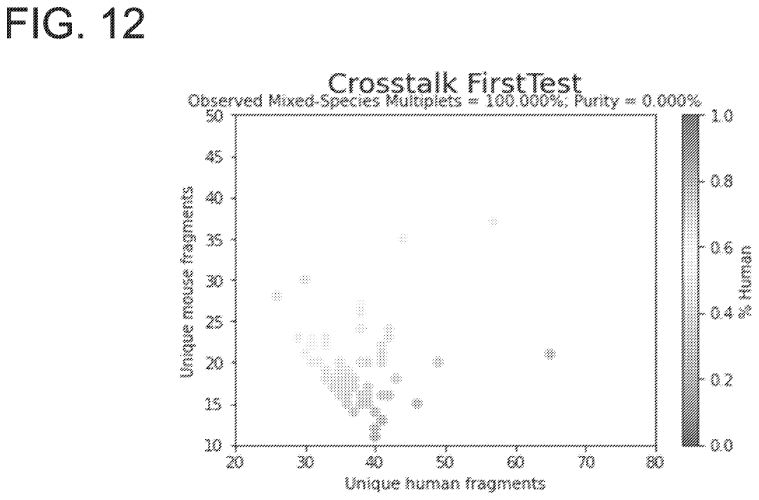

Sequenced cell barcoded libraries were analyzed for mouse human read contribution. Result: As shown in FIG. 12, little separation between mouse and human cells were observed, indicating optimization of the protocol was needed.

### Experiment 2:

To determine why little separation between mouse and human cells were observed in Experiment 1 of Example 1, the inventors sought out to modify the cell fixation protocol prior to performing library preparation of precursor libraries and cell barcoding.

Instead of using an IncellMax (IncellDx) fixative as described in Protocol A, paraformaldehyde (PFA) was used as the fixative in experiment 2, and Experiment 1 was repeated using PFA as the fixative. See Protocol B, below:

### Protocol B—Modified Cell Fixation

This fixation protocol (Protocol B), is same as Protocol A, except instead of IncellMax, Paraformaldehyde (PFA) is added to the cells to a final concentration of 1.6X PFA and 0.5 cells/ml.

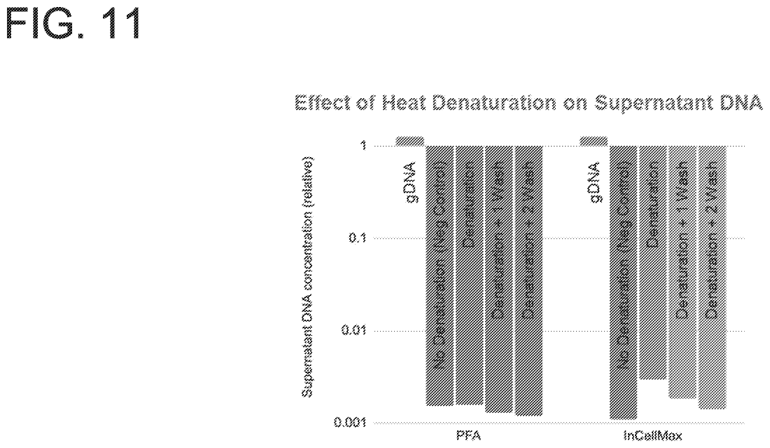

As shown in FIG. 11, it was surprisingly found that PFA was a better fixative for reducing supernatant DNA that occurs after a heat denaturing step during library preparation of precursor libraries. Amount of DNA within the supernatant was determined via qPCR with raw-unpurified supernatants.

Due to non-optimal buffer conditions, total concentration of DNA was not able to be determined, but relative amounts were determined between like samples. Measuring Supernatant DNA After DNA Accessibility. PFA fixation (Protocol B) had less supernatant DNA than InCellMax samples after denaturation. 100 ng gDNA was diluted in 37 uL dPBS to match buffer conditions of the cell samples. Cell samples underwent DNA Accessibility conditions as indicated, where “No Denaturation” sample was placed on ice for the 20 min denaturation step and the washes were performed by raising volume and removing all but 5 uL after centrifugation. All samples were eluted to 37 uL before qPCR.

### Example 2: Single Reaction Cell Barcoding Amplification

In order to perform single stream cell barcoding, cell barcoding oligos need to be amplified in the same buffer as PCR amplification of the precursor library. Thus, to identify a condition suitable for this dual purpose reaction, a number of buffers using different PCR polymerase enzymes and isopolymerase enzymes were tested to assess compatibility with amplification and droplet formation.

### Experiment 1:

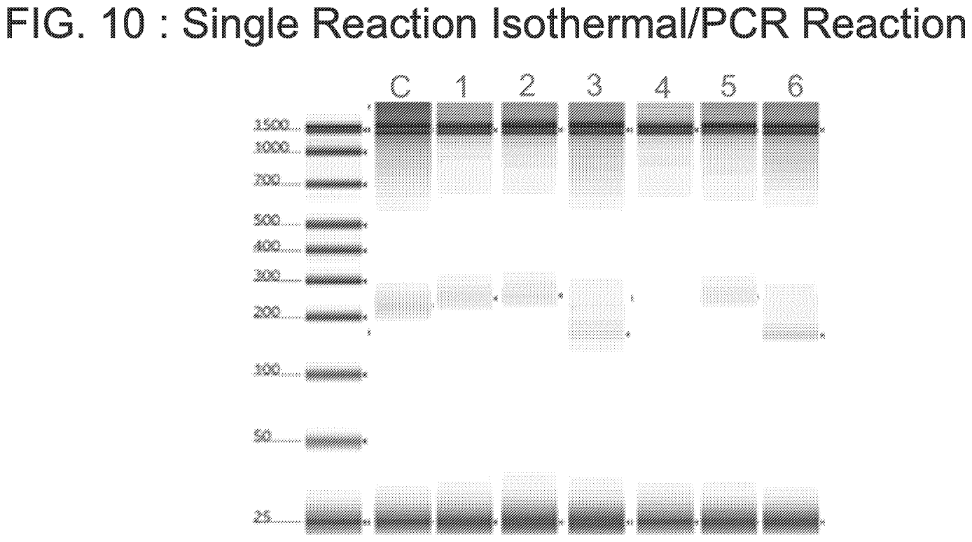

All samples used a 10 ng purified precursor library containing R1 and R2 adapter sequences ligated on as the template for PCR. The tapestation results as shown in FIG. 10 show (C) Control samples that were amplified using NEBNext Q5 enzyme (PCR enzyme) with P5/P7 indexing primers, which amplify the precursor library; Columns 1,2,3 of FIG. 12 used WM Equinox 5X Buffer and WM Equinox Polymerase (PCR enzyme) for the reaction buffer and PCR enzyme, respectively; Columns 4,5,6 of FIG. 12 used NEB Q5 5X Reaction Buffer and NEB Q5 polymerase (PCR enzyme) for the reaction buffer and PCR enzyme, respectively; and Columns 1-6) use 0.2 pmol each P5 and P7 barcoding oligos and 40 pmol barcoding oligo amplification primer were included in each reaction.

Following preparation of the reactions, isothermal amplification was performed with cell barcoding oligonucleotides on the sample. After 30 minutes of isothermal amplification, PCR amplification was performed for 12 cycles. Samples were then purified and amplified with an additional 12 cycles using NEB Q5. Columns 1,4) also included 19.2U Bst2.0 (NEB) isothermal polymerase to the reaction. Isothermal amplification was performed at 60*C. Columns 2,5) included 15.6U Sequenase2.0 (ThermoFisher) isothermal polymerase in the reaction. Isothermal amplification was performed at 30*C. Columns 3,6) included 16 Units of Isopol BST SD+ (Articzymes) isothermal polymerase in the reaction. Isothermal amplification was performed at 30*C.

### Results:

Columns 3, 6) with Isopol SD+ did not produce appropriately sized library fragments in either Equinox buffer or Q5 Reaction Buffer.

Bst2.0 worked in the Equinox buffers, but not Q5 buffer.

Sequenase worked with both Equinox buffer and Q5 Reaction Buffer.

Therefore, the type and/or amount of buffer in combination with a specific enzyme and enzyme concentration used was shown to have an effect on amount of library production.

### Experiment 2:

None of the tested enzyme buffer combinations from Experiment 1 of Example 2, formed droplets on the Geode (Stilla), data not shown. However, testing the optimal buffer and PCR enzyme (1X Naica® multiplex PCR MIX (Stilla Technologies) for the instrument with Bst2.0, provided evidence that Bst2.0 can amplify in the required buffer for droplet formation.

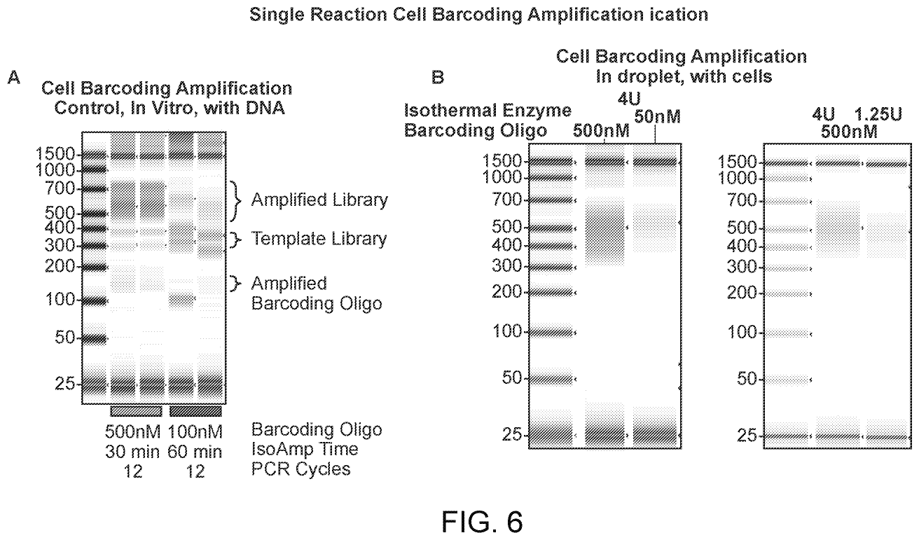

FIG. 6A showed that cell barcoding isothermal amplification and PCR amplification reactions can occur in the same buffer, in vitro and still result in cell barcoded libraries product (top and bottom lines with the two conditions). FIG. 6B shows performance of the isothermal amplification and PCR amplification reactions on precursor libraries, in situ. After recovering droplets, the droplets were amplified again and resulted in cell barcoded libraries in the expected size range. FIG. 6B shows that cell barcoding amplification and PCR amplification reactions can occur on precursor libraries in the same buffer and in droplets, resulting in recovered cell barcoded libraries. The inventors showed that cell barcoded libraries were recovered using differing amounts of barcoding oligonucleotides and differing amounts of isothermal polymerase enzymes (FIG. 6B).

In short, as shown in FIG. 6, single reaction cell barcoding works in vitro and in droplets. FIG. 6A shows single reaction cell barcode amplification works with a template library and a barcoding oligo containing a 3′ inv dT (used to prevent lengthening of the barcode oligo during isothermal amplification). This panel showed amplification of the template library and production of amplified barcoding oligo. In FIG. 6B, reaction conditions were then tested in droplets, some of which contain in situ library prepped cells (precursor libraries) using protocol K with modification to support the indicated amount of isothermal enzyme (4U or 1.25U) and barcoding oligonucleotide (500 nM or 50 nM). The barcoding oligo used was un-modified (no 3′ inv dT) and a second amplification using P5/P7 was required after droplet recovery to observe accurate insert fragment size.

Result: The present inventors found that 500 nM and 50 nM amplified libraries inside of droplets, and amplification worked around 3.84U Bst2.0 (8000U/ml stock from NEB) (libraries were observed using 1.25U using the same product). The storage buffer Bst2.0 was prepared in became detrimental to droplet formation above 3.84U Bst2.0.

Using a higher concentrated enzyme (120000 U/ml Bst2.0) amplification was observed up to 19U Bst2.0 without protocol modifications.

### Protocol K: Single Stream Cell Barcoding

A master mix containing 1X PCR buffer and Enzyme (Stilla Technologies), 4U of Bst2.0 (NEB), AOPI dye (Nexcelom), and extra dNTP (28nmol extra) and MgSO4 (0.12 umol extra) was prepared. Note the 1X PCR buffer contained, along with other components, an undisclosed concentration of dNTP and MgSO4. A second Master Mix of Cells, barcoding oligonucleotides (0.05pmol, ea) and amplification oligos (40pmol) was prepared. These two master mixes were combined before loading the Geode (Stilla Technologies). Droplet formation and PCR Amplification were performed using the following cycling conditions:

| | | Temp | | |
|---|---|---|---|---|
| | | Bst 2.0 | | |
| Step | Cycle | (° C.) | Time | |
| Partition | 1 | 40 | — | |
| Isothermal | 1 | 60 | 30 | min |
| Amplification | | | | |
| Initial | 1 | 95 | 10 | min |
| Denaturation | | | | |
| PCR Cycling | 45 | 95 | 10 | sec |
| | | 58 | 30 | sec |

| | | | |
|---|---|---|---|
| Close Cycle | 1 | — | — |
| Release | 1 | — | — |

Recovery was performed using the a commercially available Geode recovery protocol from Stilla's instrument. DNA libraries were cleaned up using Chloroform and PCR amplification performed with P5/P7 amplification primers (Watchmaker Genomics).

### Cell Barcoding Oligo Sequences:

P5CBO-
(SEQ ID NO: 1)
5′-GTCGTGTAGGGAAAGAGTGTAANNNNNGTNNNNNGTNNNNNGTNNNN
NCCGTGTAGATCTCGGTGGTCGCCGTATCATTAAAAAAAAAAAAAAAAAA
AAA-3′
p7CBO-
(SEQ ID NO: 2)
5′-ACACGTCTGAACTCCAGTCACNNNNNACNNNNNACNNNNNACNNNN
NATCTCGTATGCCGTCTTCTGCTTGAAAAAAAAAAAAAAAAAAAAA-3′
Amplification Oligo-
(SEQ ID NO: 3)
5′-TTTTTTTTTTTTTTTTTTTT-3′

### Experiment 3:

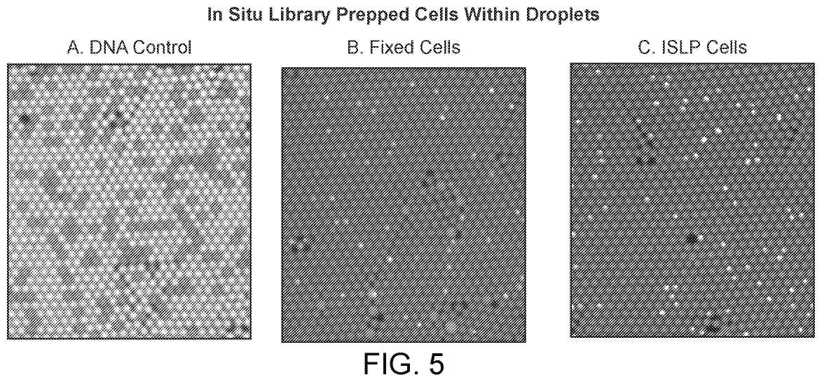

FIG. 5 provides images of droplets were taken for A) Control DNA, B) cells which have only undergone fixation, and C) cells that have had libraries prepared within them using the in situ library prep protocol (precursor libraries) as described in Protocol D and in PCT Patent Application Publication Nos. WO2022036273 and WO2022192603. FIG. 5 shows a subset of the flow cell and indicated that cells fit within the droplets, and in situ library prep (precursor libraries) does not affect the loading of droplets.

In all cases, reactions were prepared with ¼ recommended AOPI dye (Nexcelom) in a Master Mix containing a final concentration of 1X Naica® multiplex PCR MIX (Stilla Technologies). PCR Amplification occurred after segmentation, before imaging. A) IQOQ probes (Stilla Technologies) were included, with [10 ng DNA]. B-C) 15/17 Indexing Primers (IDT) were included, with 1000 cells. Shown are images from the “blue” channel (Ex 415-480 nm; Em 495-520 nm), which recognizes acridine orange bound to dsDNA.

### Example 3: Optimization of In Situ Library Prep Using Droplet Segmentation for Cell Barcoding

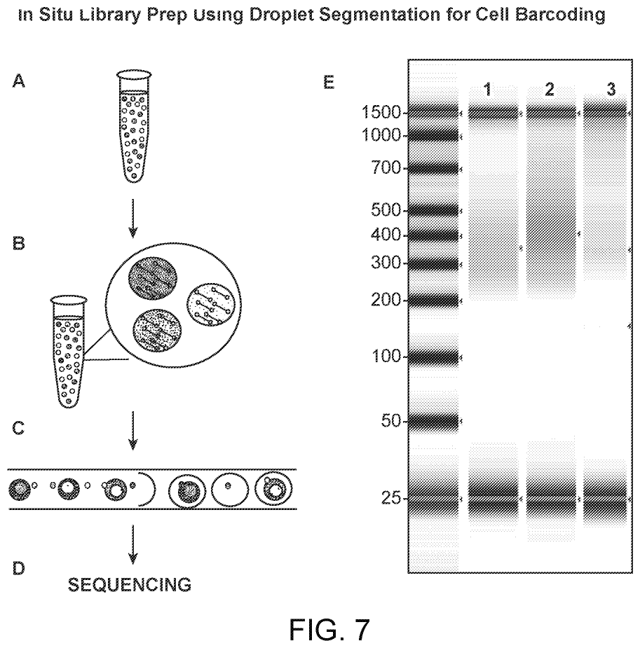

FIG. 7A-7D shows in-droplet cell barcoding using in situ library prepped cells.

A single cell suspension (FIG. 7A) was used to perform in situ library preparation of Samples 1-3 to create precursor libraries. Samples 1-2 used fixed and permeabilized cells, mixed at different points in the reaction. Sample 3 used nuclei. Library preparation steps including enzymatic fragmentation, End Repair, A-tailing, and Ligation were performed in situ (FIG. 7B). All samples included a mix of 50% human and 50% mouse cells/nuclei. Complete protocols for each sample are as follows:

### Sample 1 (Cells):

Sample 1, was a positive control for cross-talk, and human and mouse cells were mixed immediately before droplet segregation. GM12878 cells and EL4 cells were fixed according to protocol B (see experiment 2), independently. Then they were made into precursor libraries using 100K cells that were prepared following protocol D (see experiment 1) with the following modifications: Cells were washed 2X after ligation and then mixed at a 50% ratio after precursor library prep.

### Sample 2 (Cells):

GM12878 cells and EL4 cells were fixed according to protocol B (see experiment 2), independently. Then they were mixed at a ratio of 50% Human to 50% Mouse, before preparing precursor libraries using 100K cells were prepared following protocol D (see experiment 1) with the following modifications: Cells were washed 2 times after DNA accessibility by bringing volume up to 200 uL with PBS and removing all but 5 uL and resuspending in PBS accordingly. Cells were additionally washed between End Repair and ligation, cells were resuspended in PBS diluted Frag/AT buffer to ensure reaction conditions do not change from SOP. Cell were then washed 2X after ligation.

### Sample 3 (Nuclei):

In sample 2, cells were mixed and 2 washes were performed before library preparation for creating precursor libraries. GM12878 cells and EL4 cells were processed into nuclei using protocol C (as follows), independently. Then they were made into precursor libraries using 100K nuclei following protocol D (see experiment 1) with the following modifications. No DNA accessibility step was performed as the protocol c includes this step. Cells were mixed after fragmentation before ligation.

### Protocol C: Nuclei Prep and Nuclei Fixation:

Resuspend cells in NIB-L (0.5% Triton X-100; 10 mM TrisHCl, ph7.4; 10 mM NaCl; 3 mM MgCl2; 1X protease inhibitors (Roche complete tablet)) to 10K cells/uL incubate on ice for 3 minutes, then add NIB-O (10 mM TrisHCl, ph7.4; 10 mM NaCl; 3 mM MgCl2; 1X protease inhibitors (Roche complete tablet)) to a final concentration of 2.5K cells/uL. Centrifuge at 500×g for 5 minutes. Wash with NIB-O 2 times. Resuspend cells in NIB-O+12.5 mM LIS to a concentration of 10K cells/uL. Incubate on ice for 5 minutes. Add NIB-O to a final concentration of 2.5K cells/uL. Centrifuge at 1500×g for 10 minutes resuspended in dPBS to estimated concentration of 1M nuclei/ml. Fix according to Protocol B (experiment 2), where instead of cells, nuclei are used.

The resulting prepared precursor libraries (FIG. 7B) were then combined with barcoding reagents for single cell generation following protocol F (see experiment 1) using ~6000 cells or nuclei (FIG. 7C). Double stranded libraries (Samples 1, 2, and 3 of FIG. 7E) were then labeled and sequenced (FIG. 7D).

Result: FIG. 7E shows cell barcoded library yield for one example cell barcoding experiment using cell segmentation and labeling via the 10X ATAC seq Single Cell Kit. Tagmentation was not performed according to the kit, but barcoding beads and amplification primers were used. The results show that single cell barcoding is possible in partitions with in situ library prep (precursor libraries) on cells and nuclei.

### Example 4: Sequencing Analysis of Droplet Barcoded Libraries

Next, the inventors sought to improve purity of the precursor and cell barcoded libraries. Libraries generated in example 3, were sequenced on a MiSeq.

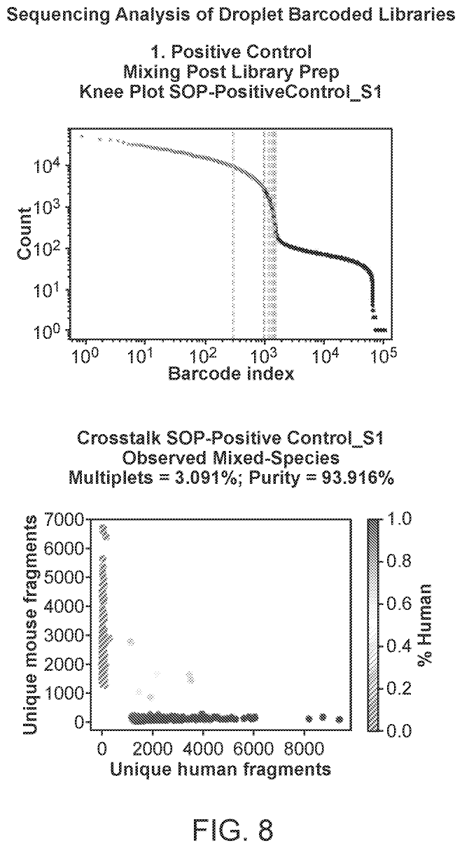

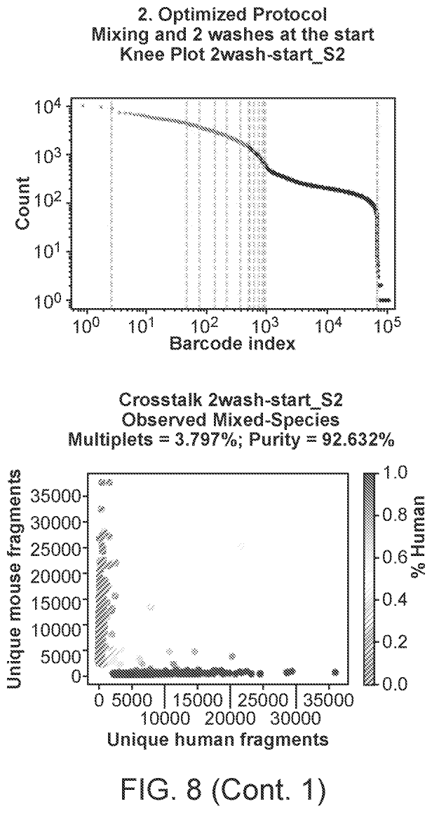

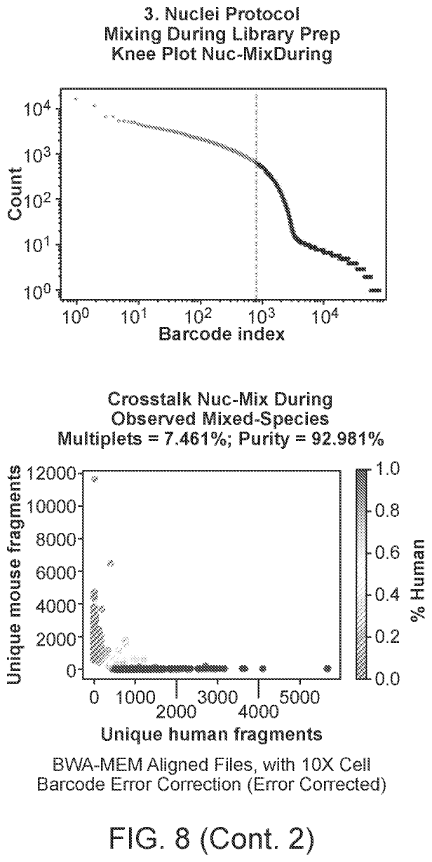

FIG. 8. Samples 1 (Positive Control, Sample 1 from experiment 4) and 2 (2wash start, sample 2 from experiment 4) and 3 (Nuc-MixDuring, sample 3 from experiment 4) from FIG. 7 were sequenced and analalyzed. Putative knee positions were identified for all samples, the knee in dark grey was selected and barcodes with more reads than the knee were defined as cells. Cells were then analyzed for human and mouse DNA content. The Positive control (FIG. 7 description) had 500 cells with a 93.9% purity and 3% multiplets. The 2wash-start sample performed nearly as well, and had 92.6% purity and 3.8% multiplets from 1000 cells. The Nuc-MixDuring sample yielded 831 cells with a purity of 92.9% and 7.5% multiplets.

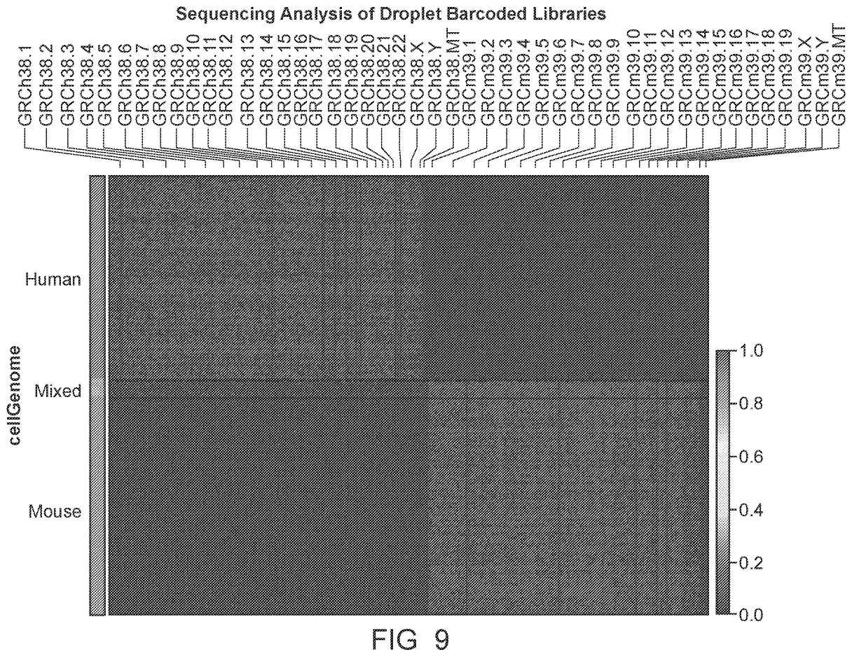

FIG. 9. Sequencing Analysis of Droplet Barcoded Libraries. For Sample 2, the 2wash-start sample, reads were binned in 1,000,000 bp regions and plotted against mouse and human genomes. Human consisted of >80% human reads, while mouse consisted of >80% mouse reads. The heat map showed good separation of mapped reads for these genomes.

### Example 5: Single Reaction Isothermal/PCR Reaction in Partitions

Next, the inventors sought to determine whether cell barcoding steps in droplets could occur in a single reaction. The present inventors surprisingly found that cell barcoding steps of isothermal amplification and PCR Amplification, in situ, could be performed in droplets in a single reaction.

All samples used 10 ng purified precursor library containing R1 and R2 adapter sequences ligated on as the template for PCR. Column (C) on the Western Blot of FIG. 10 shows a control sample amplified using NEBNext Q5 enzyme and P5/P7 indexing primers. Columns 1,2,3) of FIG. 10 used WM Equinox 5X Buffer and WM Equinox Polymerase for the reaction buffer and PCR enzyme, respectively. Columns 4,5,6) of FIG. 10 used NEB Q5 5X Reaction Buffer and NEB Q5 polymerase for the reaction buffer and PCR enzyme, respectively. Columns 1-6) includes 0.2 pmol of each P5 and P7 barcoding oligos and 40 pmol barcoding oligo amplification primer in each reaction.

After 30 minutes of isothermal amplification, PCR amplification was performed for 12 cycles, samples were purified an amplified an additional 12 cycles using NEB Q5. Columns 1,4) of FIG. 10 also included 19.2U Bst2.0 (NEB) to the reaction. Isothermal amplification was performed at 60*C. Columns 2,5) also included 15.6U Sequenase2.0 (ThermoFisher) in the reaction. Isothermal amplification was performed at 30*C. Columns 3,6) also included 16 Units of Isopol SD+ (Articzymes) in the reaction. Isothermal amplification was performed at 30*C.

Results: FIG. 10 shows that isothermal amplification and PCR reaction occurring on cells in droplets can occur in a single reaction container without compromising cell barcoded library yield.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of preparing in situ libraries, the method comprising:
(a) preparing, in situ, precursor libraries from a sample comprising a cell population to produce a precursor library within each cell, comprising nucleic acid fragments with a genomic region of interest and adapter sequences;
(b) preparing a mixture in one or more containers comprising one or more of: the cell population, cell barcoding oligonucleotides, and amplification reagents;
(c) partitioning the mixture into a plurality of partitions, wherein at least some partitions contain:
a cell from the cell population comprising the precursor library, and
a plurality of cell barcoding oligonucleotides;
(d) barcoding the precursor libraries in the plurality of partitions by:
i. amplifying the cell barcoding oligonucleotides in the at least some partitions to produce cell barcoding primers;
ii. amplifying the precursor libraries in partitions with the cell barcoding primers to produce cell barcoded libraries; and
(e) isolating the cell barcoded libraries.

2. The method of claim 1, wherein the partition in the plurality of partitions is a droplet.

3. The method of claim 1, wherein the partition in the plurality of partitions is an emulsion.

4. The method of claim 1, wherein the partition in the plurality of partitions is a container.

5. The method of claim 4, wherein the container is a well.

6. The method of claim 1, wherein the well is a microwell or nanowell.

7. The method of claim 1, wherein the partition is a hydrogel.

8. The method of claim 1, wherein the partition is poly(ethylene glycol) (PEG).

9. The method of any one of claims 1-8, wherein said partitioning the mixture into the plurality of partitions occurs within a single stream from a single container.

10. The method of any one of claims 1-8, wherein said partitioning the mixture into the plurality of partitions occurs after the mixture from two containers are combined.

11. The method of any one of claims 1-10, wherein each partition in the plurality of partitions holds a volume ranging from 0.1 to 5 nanoliters.

12. The method of claim 11, wherein each partition in the plurality of partitions has a volume ranging from 0.1 to 1 nanoliters.

13. The method of claim 2, wherein the droplet has a volume ranging from 0.1 to 5 nanoliters.

14. The method of any one of claims 1-13, wherein the at least some partitions comprise at least 80% partitions containing cells from the cell population comprising the precursor library and the plurality of cell barcoding oligonucleotides.

15. The method of claim 14, wherein the at least some partitions comprise at least 70% partitions containing cells from the cell population comprising the precursor library and the plurality of cell barcoding oligonucleotides.

16. The method of claim 15, wherein at least some partitions comprise at least 60% partitions containing cells from the cell population comprising the precursor library and the plurality of cell barcoding oligonucleotides.

17. The method of claim 16, wherein at least some partitions comprise at least 50% partitions containing cells from the cell population comprising the precursor library and the plurality of cell barcoding oligonucleotides.

18. The method of any one of claims 1-13, wherein the at least some partitions comprise at least 30% partitions containing cells from the cell population comprising the precursor library and the plurality of cell barcoding oligonucleotides.

19. The method of any one of claims 1-13, wherein the at least some partitions comprise at least 20% partitions containing cells from the cell population comprising the precursor library and the plurality of cell barcoding oligonucleotides.

20. The method of any one of claims 1-17, wherein said amplifying the cell barcoding oligonucleotides comprises amplifying via isothermal amplification.

21. The method of any one of claims 1-20, wherein amplifying the precursor libraries in partitions with the cell barcoding primers to produce cell barcoded libraries comprises PCR amplification.

22. The method of claim 21, wherein the PCR amplification is digital PCR amplification.

23. The method of any one of claims 1-22, wherein at least one of the amplification reagents comprise a buffer.

24. The method of any one of claims 1-23, wherein at least one of the amplification reagents comprise a PCR polymerase enzyme.

25. The method of any one of claims 1-24, wherein at least one of the amplification reagents comprise an isothermal polymerase enzyme.

26. The method of any one of claims 1-25, wherein at least one of the amplification reagents comprise one or more polymerase enzymes selected from: Klenow Fragment (Exo-), Bsu Large Fragment, Bst DNA polymerase, Bst2.0, Sequenase, Bsm DNA Polymerase, EquiPhi29, and Phi29 DNA polymerase.

27. The method of any one of claims 1-23, wherein the at least one amplification reagents comprises one or more enzymes selected from: DNA polymerase, RNA polymerase, nicking enzyme, a Bst2.0 polymerase, a Phi29 polymerase, and a DNA ligase.

28. The method of any one of claims 1-27, wherein the at least one amplification reagents comprise dNTP and MgSO4.

29. The method of any one of claims 1-28, wherein the at least one amplification reagents comprise amplification oligonucleotides.

30. The method of claim 23, amplifying via isothermal amplification and amplifying via PCR amplification in the partition occur in a single reaction container comprising the buffer.

31. The method of claim 24, wherein the buffer is not aspirated, washed, or modified between isothermal amplification and PCR amplification steps.

32. The method of any one of claims 20-31, wherein isothermal amplification and PCR amplification occurs in a single reaction container.

33. The method of any one of claims 20-32, wherein the method further comprises, before barcoding the precursor libraries in the plurality of partitions, lysing the cell in each cell population.

34. The method of any one of claims 20-32, wherein the method further comprises, after barcoding the precursor libraries in the plurality of partitions, lysing the cell in each cell population.

35. The method of any one of claims 1-34, wherein preparing precursor libraries comprises:
performing, in each cell of the cell population, an enzymatic fragmentation reaction to form nucleic acid fragments;
ligating, in each cell, the nucleic acid fragments to adapter sequences sequences in situ to create a precursor library comprising ligated nucleic acid fragments.

36. The method of claim 35, wherein before preparing precursor libraries, the method comprises mixing a second cell population with the cell population to create a cell population mixture.

37. The method of claim 36, wherein the method comprises, before enzymatic fragmentation, the method comprises fixing or permeabilizing the first cell population.

38. The method of claim 37, wherein the method comprises, before enzymatic fragmentation, the method comprises fixing or permeabilizing the second cell population.

39. The method of any one of claims 36-38, wherein before preparing precursor libraries, the method comprises mixing a fixed or permeabilized second cell population with the fixed or permeabilized cell population to create a cell population mixture.

40. The method of claim 35, further comprising fixing or permeabilizing the cell population mixture before preparing precursor libraries.

41. The method of any one of claims 35-40, wherein the method further comprises performing a heat denaturation step on the cells before enzymatic fragmentation.

42. The method of claim 41, further comprising washing the cell population mixture after the heat denaturation step with a buffer.

43. The method of claim 41, further comprising at least a second washing step to wash the population mixture with the buffer.

44. The method of claim 35, wherein before mixing the second cell population with the first cell population to create the cell population mixture:
introducing a first set of unique well identifiers (UWIs) to the first population of cells;
introducing a second set of UWIs to the second population of cells;
wherein the first set of UWIs comprise one or more barcode reads and a non-genomic fragment and the second set of UWIs comprise one or more barcode reads and a non-genomic fragment;
preparing, in situ, the precursor library from the first and second population of cells to produce the precursor library, the precursor library comprising nucleic acid fragments comprising a genomic region of interest from the first and second cell populations and one or more adapter sequences
barcoding, in situ, the precursor library and the first and second set of UWIs to produce:
a cell barcoded precursor library,
a first and second set of cell barcoded UWIs;
isolating the cell barcoded precursor library and cell barcoded UWIs;
sequencing the cell barcoded precursor library and cell barcoded UWIs; and
analyzing sequencing reads to identify: cells belonging to the first population of cells and cells belonging to the second population of cells based on the UWI.

45. The method of claim 44, wherein analyzing comprises analyzing the cell barcoded UWI by the non-genomic fragment of the UWI.

46. The method of any one of claims 1-45, wherein said isolating the cell barcoded libraries comprises recovering amplicons from the cell barcoded libraries of the at least some partitions.

47. The method of claim 46, wherein isolating the cell barcoded libraries comprises breaking the partition.

48. The method of claim 46, wherein isolating the cell barcoded libraries comprises lysing the cell in the partition.

49. The method of any one of claims 1-48, wherein the concentration of barcoding oligonucleotides in the at least some partitions ranges from 25 to 600 pM.

50. The method of any one of claims 1-48, wherein the concentration of barcoding oligonucleotides in the at least some partitions ranges from 45 pM to 550 pM.

51. The method of any one of claims 1-48, wherein the concentration of barcoding oligonucleotides in the at least some partitions ranges from 50 nM to 500 nM.

52. The method of claim 1, wherein the concentration of barcoding oligonucleotides comprises 5 pM to 500 pM, or, wherein the amount of barcoding oligonucleotides in each partition containing the cell ranges from 50 barcoding oligonucleotides to 8.5 million barcoding oligonucleotides.

53. A method for cellular sample pooling, the method comprising:
introducing a first set of unique well identifiers (UWIs) to a first population of cells, the first set of UWIs comprising one or more barcode reads and a non-genomic fragment;
introducing a second set of UWIs to a second population of cells, the second set of UWIs comprising one or more barcode reads and a non-genomic fragment;
mixing the first and second population of cells;
preparing, in situ, a precursor library from the first and second population of cells to produce the precursor library, the precursor library comprising nucleic acid fragments comprising a genomic region of interest and one or more adapter sequences
barcoding, in situ, the precursor library and the first and second set of UWIs to produce:
a cell barcoded precursor library,
a first and second set of cell barcoded UWIs;
isolating the cell barcoded precursor library and cell barcoded UWIs;
sequencing the cell barcoded precursor library and cell barcoded UWIs; and
analyzing sequencing reads to identify: cells belonging to the first population of cells and cells belonging to the second population of cells based on the UWI.

54. A kit comprising:
one or more microfluidic chips;
reagents for preparing precursor libraries in situ comprising:
a fragmentation enzyme and buffer for performing an enzymatic fragmentation reaction to form one or more nucleic acid fragments within a cell;
an End repair and A tail (ERA) master mix and buffer for performing an end-repair and A-tailing reaction on the one or more nucleic acid fragments;
a ligation enzyme and buffer;
adapter sequences, wherein the ligation enzyme and buffer, and adapter sequences are capable of ligating, in each cell, the nucleic acid fragments to the adapter sequences in situ to create a ligated library comprising ligated nucleic acid fragments;
amplification primers for amplifying the ligated nucleic acid fragments to form amplicon products;
a polymerase chain reaction (PCR) enzyme master mix comprising one or more of: an enzyme, a buffer, or an enzyme and a buffer;
in an amount sufficient to prepare a precursor library in situ;
reagents for cell barcoding comprising:
cell barcoding oligonucleotides comprising:
an oligonucleotide, wherein the oligonucleotide comprises:
an amplification sequence, and
a consensus region that is complementary to a target sequence of a nucleic acid fragment; and
a second oligonucleotide, wherein the second oligonucleotide comprises:
a second amplification sequence,
a second consensus region that complementary to a target sequence of a nucleic acid fragment;
amplification reagents comprising:
a first amplification primer comprising a nucleotide sequence that is complementary to the amplification sequence on the oligonucleotide;
a second amplification primer comprising a nucleotide sequence that is complementary to the second amplification sequence on the second oligonucleotides; and
an isothermal amplification polymerase;
instructions for carrying out the precursor library preparation in situ;
instructions for preparing a mixture in one or more containers comprising one or more of: the cell population, cell barcoding oligonucleotides, and amplification reagents;
instructions for partitioning the mixture into a plurality of partitions;
instructions for barcoding the precursor libraries in the plurality of partitions
instructions for isolating the cell barcoded libraries; and
instructions for sequencing the cell barcoded libraries.

55. The kit of claim 54, wherein instructions for carrying out the precursor library comprises the following steps:
performing, in each cell of the cell population, an enzymatic fragmentation reaction to form nucleic acid fragments;
ligating, in each cell, the nucleic acid fragments to adapter sequences sequences in situ to create a precursor library comprising ligated nucleic acid fragments.

56. The kit of any one of claims 54-55, wherein the instructions for preparing the mixture comprises the following steps:
preparing the mixture in one or more containers comprising one or more of: the cell population, cell barcoding oligonucleotides, and amplification reagents

57. The kit of any one of claims 54-56, wherein instructions for partitioning the mixture into a plurality of partitions comprises the following steps:
partitioning the mixture into a plurality of partitions, wherein at least some partitions contain:
a cell from the cell population comprising the precursor library, and
a plurality of cell barcoding oligonucleotides.

58. The kit of any one of claims 54-57, wherein instructions for barcoding the precursor library in a plurality of partitions comprises the following steps:
barcoding the precursor libraries in the plurality of partitions by:
amplifying the cell barcoding oligonucleotides in the at least some partitions to produce cell barcoding primers;
amplifying the precursor libraries in partitions with the cell barcoding primers to produce cell barcoded libraries; and

59. The kit of any one of claims 54-58, further comprising one or more buffers.

60. The kit of any one of claims 54-59, further comprising a cell lysis buffer.

61. The kit of any one of claims 54-60, wherein the amplification primers comprise barcoding primers, sequencing primers, or a combination thereof.

62. The kit of any one of claims 54-61, wherein the kit further comprises protease K.

63. The kit of any one of claims 54-63, wherein the kit further comprises barcoding primers, and a second PCR Enzyme master mix comprising one or more of: an enzyme, a buffer, or an enzyme and a buffer.

64. The kit of any one of claims 54-63, wherein the kit further comprises a lytic enzyme.

65. The kit of any one of claims 54-64, further comprising a thermocycler.

66. The kit of any one of claims 54-65, further comprising a partitioning instrument for partitioning cells, cell barcoding oligonucleotides, or a combination of cells and cell barcoding oligonucleotides.

67. The kit of any one of claims 54-66, further comprising an instrument comprising a thermocycler and a partition engine.

68. The kit of any one of claims 54-67, wherein the partition engine is configured to hold one or more microfluidic chips.

69. The kit of any one of claims 54-68, wherein the one or more microfluidic chips is configured to hold a sample comprising one or more cells in the cell population, amplification reagents, and one or more cell barcoding oligonucleotides.
